(19) United States
(12) Patent Application Publication
(10) Pub. No.: US 2022/0399129 A1
(43) Pub. Date: Dec. 15, 2022

(54) SYSTEMS AND METHODS FOR TERRAFORMING

(71) Applicant: Flagship Pioneering Innovations VI, LLC, Cambridge, MA (US)

(72) Inventors: Fabian Alexander WOLF, Munich (DE); Inna LIPCHINA, Cambridge, MA (US); Xiaoji SUN, Cambridge, MA (US); Nicholas McCartney PLUGIS, Cambridge, MA (US)

(21) Appl. No.: 17/841,516

(22) Filed: Jun. 15, 2022

Related U.S. Application Data
(60) Provisional application No. 63/210,710, filed on Jun. 15, 2021.

(51) Int. Cl. G16H 50/50; G16B 25/10

(52) U.S. Cl. CPC G16H 50/50; G16B 25/10

(57) ABSTRACT

Systems and methods for associating cellular constituents with a cellular process of interest are provided. Constituent vectors comprising abundances for a first plurality of cells representing annotated cell states are formed and used to obtain a latent representation of constituent modules having subsets of constituents. A constituent count data structure comprising abundances of the constituents for a second plurality of cells representing covariates of interest is obtained. An activation data structure is formed by combining the latent representation and the constituent count data structure, using constituents as a common dimension. A model is trained using a difference between the predicted and actual absence or presence of each covariate in each cellular constituent module represented in the activation data structure, thus adjusting covariate weights indicating a correlation between covariates and constituent modules across the activation data structure. The covariate weights are used to identify constituent modules associated with covariates of interest.

### CROSS REFERENCE TO RELATED APPLICATIONS

This application claims priority to U.S. Provisional Patent Application Nos. 63/210,710, entitled “SYSTEMS AND METHODS FOR TERRAFORMING,” filed Jun. 15, 2021; and 63/210,679, entitled “COMPUTATIONAL MODELING PLATFORM,” filed Jun. 15, 2021, each of which is hereby incorporated by reference in its entirety.

### TECHNICAL FIELD

The present invention relates generally to systems and methods for associating cellular constituents with cellular processes.

### BACKGROUND

The study of cellular mechanisms is important for understanding disease.

Biological tissues are dynamic and highly networked multicellular systems. Dysfunction in subcellular networks in specific cells shift the entire landscape of cell behaviors and leads to disease states. Existing drug discovery efforts seek to characterize the molecular mechanisms that cause cells to transition from healthy to disease states, and to identify pharmacological approaches to reverse or inhibit these transitions. Past efforts have also sought to identify molecular signatures characterizing these transitions, and to identify pharmacological approaches that reverse these signatures.

Molecular data on bulk collections of cells, in tissues or cells enriched by surface markers, mask the phenotypic and molecular diversity of individual cells in a population. The heterogeneity of cells in these bulk collections of cells causes the results of current efforts aimed at elucidating disease-driving mechanisms to be misleading or even wholly incorrect. New approaches, such as single-cell RNA sequencing, can characterize individual cells at the molecular level. These data provide a substrate for understanding varied cell states at higher resolution and reveal the rich and remarkable diversity of states that cells possess.

Significant challenges exist when interpreting single cell data, namely the sparsity of these data, overlooking the presence of molecules present in cells, and noise, with uncertainty in the accuracy of these molecular measurements. Accordingly, new approaches are required to derive insight into pharmacological approaches for controlling individual cell state, and to correspondingly resolve disease.

In addition, complex diseases often cannot be broken down to a single or a few molecular targets. Though recent advances in high-throughput imaging technology and high-throughput screening for in vitro disease models have somewhat overcome a dependence on molecular targets, the gap between a hit generated through in-vitro-based screening and an efficacious drug is immense, and translating a hit obtained from such phenotypic drug discovery into an efficacious drug often necessitates a return to the slow and inefficient molecular-target-based drug discovery approach.

Given the above background, what is needed in the art are systems and methods for discovery of molecular targets for drug discovery.

### SUMMARY

The present disclosure addresses the above-identified shortcomings. The present disclosure addresses these shortcomings, at least in part, with cellular constituent data (e.g., abundances of genes) corresponding to annotated cell states and covariates of interest (e.g., phenotypes, diseases, and/or cellular processes of interest), and using latent representations and machine learning to determine correlations between modules (e.g., subsets) of cellular constituents and the cellular process of interest. In particular, the present disclosure provides systems and methods for elucidating molecular mechanisms underlying various cell states, such as disease.

One aspect of the present disclosure provides a method of associating a plurality of cellular constituents with a cellular process of interest. The method comprises, at a computer system comprising a memory and one or more processors, obtaining one or more first datasets in electronic form. The one or more first datasets comprise or collectively comprise, for each respective cell in a first plurality of cells, where the first plurality of cells comprises twenty or more cells and collectively represents a plurality of annotated cell states, for each respective cellular constituent in a plurality of cellular constituents, where the plurality of cellular constituents comprises 50 or more cellular constituents, a corresponding abundance of the respective cellular constituent in the respective cell. The method thereby comprises accessing or forming a plurality of vectors, each respective vector in the plurality of vectors (i) corresponding to a respective cellular constituent in the plurality of constituents and (ii) comprising a corresponding plurality of elements, each respective element in the corresponding plurality of elements having a corresponding count representing the corresponding abundance of the respective cellular constituent in a respective cell in the first plurality of cells.

The plurality of vectors is used to identify each cellular constituent module in a plurality of cellular constituent modules, each cellular constituent module in the plurality of cellular constituent modules including a subset of the plurality of cellular constituents. The plurality of cellular constituent modules is arranged in a latent representation dimensioned by (i) the plurality of cellular constituent modules and (ii) the plurality of cellular constituents or a representation thereof. The plurality of cellular constituent modules comprises more than ten cellular constituent modules.

The method further includes obtaining one or more second datasets in electronic form. The one or more second datasets comprise or collectively comprise, for each respective cell in a second plurality of cells, where the second plurality of cells comprises twenty or more cells and collectively represents a plurality of covariates possibly informative of the cellular process of interest, for each respective cellular constituent in the plurality of cellular constituents, a corresponding abundance of the respective cellular constituent in the respective cell. The method thereby obtains a cellular constituent count data structure dimensioned by (i) the second plurality of cells and (ii) the plurality of cellular constituents or the representation thereof.

An activation data structure is formed by combining the cellular constituent count data structure and the latent representation using the plurality of cellular constituents or the representation thereof as a common dimension, where the activation data structure comprises, for each cellular constituent module in the plurality of cellular constituent modules and for each cellular constituent in the plurality of cellular constituents, a respective activation weight.

A model is trained using a difference between (i) a prediction of an absence or presence of each covariate in the plurality of covariates in each cellular constituent module represented in the activation data structure upon input of the activation data structure into the model and (ii) actual absence or presence of each covariate in each cellular constituent module, where the training adjusts a plurality of covariate weights associated with the model responsive to the difference. The plurality of covariate weights comprises, for each respective cellular constituent module in the plurality of cellular constituent modules, for each respective covariate, a corresponding weight indicating whether the respective covariate correlates, across the activation data structure, with the respective cellular constituent module.

The method further comprises identifying, using the plurality of covariate weights upon training the model, one or more cellular constituent modules in the plurality of cellular constituent modules that is associated with one or more covariates in the plurality of covariates, thereby associating a plurality of cellular constituents with the cellular process of interest.

Another aspect of the present disclosure provides a method of associating a plurality of cellular constituents with a cellular process of interest. The method comprises, at a computer system comprising a memory and one or more processors, obtaining one or more first datasets in electronic form. The one or more first datasets comprise or collectively comprise, for each respective cell in a first plurality of cells, where the first plurality of cells comprises twenty or more cells and collectively represents a plurality of annotated cell states, for each respective cellular constituent in a plurality of cellular constituents, where the plurality of cellular constituents comprises 50 or more cellular constituents, a corresponding abundance of the respective cellular constituent in the respective cell. The method thereby comprises accessing or forming a plurality of vectors, each respective vector in the plurality of vectors (i) corresponding to a respective cellular constituent in the plurality of constituents and (ii) comprising a corresponding plurality of elements, each respective element in the corresponding plurality of elements having a corresponding count representing the corresponding abundance of the respective cellular constituent in a respective cell in the first plurality of cells.

The plurality of vectors is used to identify each cellular constituent module in a plurality of cellular constituent modules, each cellular constituent module in the plurality of cellular constituent modules including a subset of the plurality of cellular constituents, and where the plurality of cellular constituent modules comprises more than ten cellular constituent modules.

For each respective cellular constituent module in the plurality of cellular constituent modules, the identity of each cellular constituent in the respective cellular constituent module is used to associate the respective cellular constituent module with a known cellular pathway, a biological process, a transcription factor, a cell receptor, or a kinase using a contextualization algorithm.

The method further comprises pruning the activation data structure by removing from the activation data structure one or more cellular constituent modules that fail to associate with a known cellular pathway, a biological process, a transcription factor, a cell receptor, or a kinase that is implicated in the cellular process of interest thereby identifying one or more cellular constituent modules in the plurality of cellular constituent modules that is associated with the cellular process of interest and, from the one or more cellular constituent modules, the plurality of cellular constituents associated with the cellular process of interest.

Another aspect of the present disclosure provides a method of training a model to identify a set of cellular constituents associated with a cellular process of interest. The method comprises (e.g., at a computer system comprising a memory and one or more processors) (A) obtaining one or more first datasets (e.g., in electronic form). The one or more first datasets individually or collectively comprise, for each respective cell in a first plurality of cells (e.g., where the first plurality of cells comprises twenty or more cells and collectively represents a plurality of annotated cell states), for each respective cellular constituent in a plurality of cellular constituents (e.g., where the plurality of cellular constituents comprises 50 or more cellular constituents), a corresponding abundance of the respective cellular constituent in the respective cell, thereby accessing or forming a plurality of vectors. Each respective vector in the plurality of vectors (i) corresponds to a respective cellular constituent in the plurality of constituents and (ii) comprises a corresponding plurality of elements. Each respective element in the corresponding plurality of elements has a corresponding count representing the corresponding abundance of the respective cellular constituent in a respective cell in the first plurality of cells.

The method further comprises (B) using the plurality of vectors to identify each respective cellular constituent module in a plurality of cellular constituent modules. Each respective cellular constituent module in the plurality of cellular constituent modules includes an independent subset of the plurality of cellular constituents. In some embodiments, the plurality of cellular constituent modules are arranged in a latent representation dimensioned by (i) the plurality of cellular constituent modules and (ii) the plurality of cellular constituents or a representation (e.g., dimension reduction components) thereof. In some embodiments the plurality of cellular constituent modules comprises more than ten cellular constituent modules. In some embodiments the plurality of cellular constituent modules is in fact just a single cellular constituent module. In some embodiments the plurality of cellular constituent modules is two or more cellular constituent modules.

The method further comprises (C) obtaining one or more second datasets (e.g., in electronic form). The one or more second datasets individually or collectively comprise, for each respective cell in a second plurality of cells (e.g., where the second plurality of cells comprises twenty or more cells and collectively represents a plurality of covariates associated with the cellular process of interest), for each respective cellular constituent in the plurality of cellular constituents, a corresponding abundance of the respective cellular constituent in the respective cell, thereby obtaining a cellular constituent count data structure dimensioned by (i) the second plurality of cells and (ii) the plurality of cellular constituents or the representation thereof.

The method further comprises (D) forming an activation data structure by combining the cellular constituent count data structure and the latent representation using the plurality of cellular constituents or the representation thereof as a common dimension (e.g., through matrix multiplication of this common dimension. The activation data structure comprises, for each cellular constituent module in the plurality of cellular constituent modules, for each cell in the second plurality of cells, a respective activation weight.

The method further comprise (E) training the model using, for each respective covariate in the plurality of covariates, a difference between (i) a calculated activation against each cellular constituent module represented by the model upon input of a representation of the respective covariate into the model and (ii) actual activation against each cellular constituent module represented by the model. The training adjusts a plurality of covariate parameters associated with the model responsive to each difference. Each respective covariate parameter in the plurality of covariate parameters represents a covariate in the plurality of covariates.

In some embodiments, the plurality of covariate parameters comprises, for each respective cellular constituent module in the plurality of cellular constituent modules, for each respective covariate, a corresponding covariate parameter indicating whether the respective covariate correlates, across the second plurality of cells, with the respective cellular constituent module.

In some embodiments, the method further comprises (F) identifying, using the plurality of covariate weights upon training the model, one or more cellular constituent modules in the plurality of cellular constituent modules that is associated with one or more covariates in the plurality of covariates, thereby associating each cellular constituent in the set of plurality of cellular constituents, from among the cellular constituents in the identified one or more cellular constituent modules, with the cellular process of interest.

In some embodiments, an annotated cell state in the plurality of annotated cell states is an exposure of a cell in the first plurality of cells to a compound under an exposure condition. In some such embodiments, the exposure condition is a duration of exposure, a concentration of the compound, or a combination of a duration of exposure and a concentration of the compound.

In some embodiments, each cellular constituent in the plurality of cellular constituents is a particular gene, a particular mRNA associated with a gene, a carbohydrate, a lipid, an epigenetic feature, a metabolite, a protein, or a combination thereof.

In some embodiments, each cellular constituent in the plurality of cellular constituents is a particular gene, a particular mRNA associated with a gene, a carbohydrate, a lipid, an epigenetic feature, a metabolite, a protein, or a combination thereof, the corresponding abundance of the respective cellular constituent in the respective cell in the first or second plurality of cells is determined by a colorimetric measurement, a fluorescence measurement, a luminescence measurement, or a resonance energy transfer (FRET) measurement.

In some embodiments, each cellular constituent in the plurality of cellular constituents is a particular gene, a particular mRNA associated with a gene, a carbohydrate, a lipid, an epigenetic feature, a metabolite, a protein, or a combination thereof, and the corresponding abundance of the respective cellular constituent in the respective cell in the first or second plurality of cells is determined by single-cell ribonucleic acid (RNA) sequencing (scRNA-seq), scTag-seq, single-cell assay for transposase-accessible chromatin using sequencing (scATAC-seq), CyTOF/SCoP, E-MS/Abseq, miRNA-seq, CITE-seq, or any combination thereof.

In some embodiments, the using the plurality of vectors to identify each cellular constituent module in a plurality of cellular constituent modules comprises application of a correlation model to the plurality of vectors using each corresponding plurality of elements of each vector in the plurality of vectors. In some such embodiments, the correlation model includes a graph clustering (e.g., Leiden clustering on a Pearson-correlation-based distance metric, Louvain clustering, etc.).

In some embodiments, the using the plurality of vectors to identify each cellular constituent module in the plurality of cellular constituent modules comprises a dictionary learning model that produces the representation of the plurality of cellular constituents as a plurality of dimension reduction components. In some such embodiments, the dictionary learning model is L0-regularized autoencoder.

In some embodiments, the plurality of cellular constituent modules consists of between 10 and 2000 cellular constituent modules.

In some embodiments, the plurality of cellular constituent modules consists of between 50 and 500 cellular constituent modules, between twenty and 10,000 cellular constituents, or between 100 and 8,000 cellular constituents.

In some embodiments, each cellular constituent module in the plurality of constituent modules consists of between two cellular constituents and three hundred cellular constituents.

In some embodiments, the cellular process of interest is an aberrant cell process associated with a disease, and the first plurality of cells includes cells that are representative of the disease and cells that are not representative of the disease as indicated by the plurality of annotated cell states.

In some embodiments, a respective covariate in the plurality of covariates comprises cell batch and the representation of the respective covariate is a cell batch identification.

In some embodiments, a respective covariate in the plurality of covariates comprises cell donor and the representation of the respective covariate is an identification of the cell donor or a characteristic of the cell donor.

In some embodiments, a respective covariate in the plurality of covariates comprises cell type and the representation of the respective covariate is a cell type identification.

In some embodiments, a respective covariate in the plurality of covariates comprises disease status and the representation of the respective covariate is an indication of absence or presence of the disease.

In some embodiments, a respective covariate in the plurality of covariates comprises exposure to a compound and the representation of the respective covariate is a fingerprint of the compound. In some such embodiments, the method further comprising generating the fingerprint from a chemical structure of the compound using Daylight, BCI, ECFP4, EcFC, MDL, TTFP, UNITY 2D, RNNS2S, GraphConv, fingerprint SMILES Transformer, RNNS2S, or GraphConv. In some such embodiments, the representation of the respective covariate further comprises a duration of time the respective covariate was incubated with the respective cell. In some such embodiments, the representation of the respective covariate further comprises a concentration of the respective covariate used to incubate the respective cell.

In some embodiments, the training the model (E) is performed using a categorical cross-entropy loss in a multi-task formulation, in which each covariate in the plurality of covariates corresponds to a cost function in plurality of cost functions and each respective cost function in the plurality of cost functions has a common weighting factor.

In some embodiments, the method further comprises using the identity of each cellular constituent in a cellular constituent module in the plurality of cellular constituent modules to associate the cellular constituent module with a known cellular pathway, a biological process, a transcription factor, a cell receptor, or a kinase using a contextualization algorithm. In some such embodiments, the contextualization algorithm is a gene set enrichment analysis algorithm.

In some embodiments, the method further comprises, for each respective cellular constituent module in the plurality of cellular constituent modules, using the identity of each cellular constituent in the respective cellular constituent module to associate the respective cellular constituent module with a known cellular pathway, a biological process, a transcription factor, a cell receptor, or a kinase using a contextualization algorithm. In some such embodiments, the contextualization algorithm is a gene set enrichment analysis algorithm. In some such embodiments, the method further comprising pruning the activation data structure by removing from the activation data structure one or more cellular constituent modules that fail to associate with a known cellular pathway, a biological process, a transcription factor, a cell receptor, or a kinase that is implicated in the cellular process of interest.

In some embodiments, the compound is an organic compound having a molecular weight of less than 2000 Daltons. In some embodiments, the compound is an organic compound that satisfies each of the Lipinski rule of five criteria. In some embodiments, the compound is an organic compound that satisfies at least three criteria of the Lipinski rule of five criteria.

In some embodiments, the plurality of cellular constituent modules comprises five or more cellular constituent modules, ten or more cellular constituent modules, or comprises 100 or more cellular constituent modules.

In some embodiments, the independent subset of the plurality of cellular constituents in the respective cellular constituent module comprises five or more cellular constituents.

In some embodiments, the independent subset of the plurality of cellular constituents in the respective cellular constituent module consists of between two and 20 cellular constituents in a molecular pathway associated with the cellular process of interest.

In some embodiments, the model is a regressor.

In some embodiments, the model is a logistic regression model, a neural network model, a support vector machine model, a Naive Bayes model, a nearest neighbor model, a boosted trees model, a random forest model, a decision tree model, a multinomial logistic regression model, a linear model, or a linear regression model.

In some embodiments, each respective covariate parameter in the plurality of covariate parameters represents a different covariate in the plurality of covariates.

In some embodiments, more than one covariate parameter in the plurality of covariate parameters represents a common covariate in the plurality of covariates.

In some embodiments, a corresponding abundance of the respective cellular constituent in the respective cell is determined using a cell-based assay.

In some embodiments, the first plurality of cells or the second plurality of cells comprises or consists of cells from an organ (e.g., heart, liver, lung, muscle, brain, pancreas, spleen, kidney, small intestine, uterus, or bladder).

In some embodiments, the first plurality of cells or the second plurality of cells comprises or consists of cells from a tissue (e.g., bone, cartilage, joint, tracheae, spinal cord, cornea, eye, skin, or blood vessel.

In some embodiments, the first plurality of cells or the second plurality of cells comprises or consists of a plurality of stem cells (e.g., a plurality of embryonic stem cells, a plurality of adult stem cells, or a plurality of induced pluripotent stem cells).

In some embodiments, the first plurality of cells or the second plurality of cells comprises or consists of a plurality of primary human cells (e.g., a plurality of CD34+ cells, a plurality of CD34+ hematopoietic stems, a plurality of progenitor cells (HSPC), a plurality of T-cells, a plurality of mesenchymal stem cells (MSC), a plurality of airway basal stem cells, or a plurality of induced pluripotent stem cells).

In some embodiments, the first plurality of cells or the second plurality of cells comprises or consists of a plurality of human cell lines.

In some embodiments, the first plurality of cells or the second plurality of cells comprises or consists of cells from umbilical cord blood, from peripheral blood, or from bone marrow.

In some embodiments, the first plurality of cells or the second plurality of cells comprises or consists of cells in or from a solid tissue (e.g., placenta, liver, heart, brain, kidney, or gastrointestinal tract).

In some embodiments, the first plurality of cells or the second plurality of cells comprises or consists of a plurality of differentiated cells (e.g., a plurality of megakaryocytes, a plurality of osteoblasts, a plurality of chondrocytes, a plurality of adipocytes, a plurality of hepatocytes, a plurality of hepatic mesothelial cells, a plurality of biliary epithelial cells, a plurality of hepatic stellate cells, a plurality of hepatic sinusoid endothelial cells, a plurality of Kupffer cells, a plurality of pit cells, a plurality of vascular endothelial cells, a plurality of pancreatic duct epithelial cells, a plurality of pancreatic duct cells, a plurality of centroacinous cells, a plurality of acinar cells, a plurality of islets of Langerhans, a plurality of cardiac muscle cells, a plurality of fibroblasts, a plurality of keratinocytes, a plurality of smooth muscle cells, a plurality of type I alveolar epithelial cells, a plurality of type II alveolar epithelial cells, a plurality of Clara cells, a plurality of ciliated epithelial cells, a plurality of basal cells, a plurality of goblet cells, a plurality of neuroendocrine cells, a plurality of kultschitzky cells, a plurality of renal tubular epithelial cells, a plurality of urothelial cells, a plurality of columnar epithelial cells, a plurality of glomerular epithelial cells, a plurality of glomerular endothelial cells, a plurality of podocytes, a plurality of mesangium cells, a plurality of nerve cells, a plurality of astrocytes, a plurality of microglia, or a plurality of oligodendrocytes.

In some embodiments, the set of cellular constituents consists of between 2 and 20 cellular constituents in the plurality of cellular constituent and the one or more cellular constituent modules consists of a single cellular constituent module.

In some embodiments, the set of cellular constituents consists of between 2 and 100 cellular constituents in the plurality of cellular constituent and the one or more cellular constituent modules comprises two or more cellular constituent modules.

In some embodiments, the set of cellular constituents consists of between 2 and 1000 cellular constituents in the plurality of cellular constituent and the one or more cellular constituent modules comprises five or more cellular constituent modules.

In some embodiments, the model is an ensemble model comprising a plurality of component models, and each respective component model in the plurality of component models provides a calculated activation for a different cellular constituent module in the plurality of cellular constituent modules responsive to inputting the representation of the respective covariate into the respective component model. In some such embodiments, the ensemble model includes a different component model for each cellular constituent module in the plurality of cellular constituent modules. In some such embodiments a component model in the plurality of component models is a logistic regression model, a neural network model, a support vector machine model, a Naive Bayes model, a nearest neighbor model, a boosted trees model, a random forest model, a decision tree model, a multinomial logistic regression model, a linear model, or a linear regression model.

Another aspect of the present disclosure provides a computer system having one or more processors, and memory storing one or more programs for execution by the one or more processors, the one or more programs comprising instructions for performing any of the methods and/or embodiments disclosed herein.

Another aspect of the present disclosure provides a non-transitory computer readable storage medium storing one or more programs configured for execution by a computer, the one or more programs comprising instructions for carrying out any of the methods disclosed herein.

Additional aspects and advantages of the present disclosure will become readily apparent to those skilled in this art from the following detailed description, where only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The embodiments disclosed herein are illustrated by way of example, and not by way of limitation, in the figures of the accompanying drawings. Like reference numerals refer to corresponding parts throughout the drawings.

FIGS. 1A, 1B, 1C, 1D, and 1E collectively illustrate a block diagram of an exemplary system and computing device, in accordance with an embodiment of the present disclosure.

FIGS. 2A and 2B collectively provide a flow chart of processes and features of an example method for associating a plurality of cellular constituents with a cellular process of interest, in accordance with various embodiments of the present disclosure.

FIG. 3 provides a flow chart of processes and features of an example method for associating a plurality of cellular constituents with a cellular process of interest, in accordance with various embodiments of the present disclosure.

FIG. 4 illustrates an example of a plurality of vectors of cellular constituents and an example of a latent representation of cellular constituent modules, in accordance with some embodiments of the present disclosure.

FIG. 5 illustrates an example of a cellular constituent count data structure and an example activation data structure, in accordance with some embodiments of the present disclosure.

FIG. 6 illustrates an example of a method of training a model to adjust a plurality of covariate weights, in accordance with some embodiments of the present disclosure.

FIG. 7 illustrates an example of a method for cross-system identification of shared and orthogonal cellular constituent modules, in accordance with some embodiments of the present disclosure.

FIG. 8 illustrates cellular constituent module activation in megakaryocyte and basophil differentiation and cellular constituent module activation targeted by 6 compound treatment conditions, in accordance with an embodiment of the present disclosure.

FIGS. 9A and 9B illustrate cellular constituent module activation in different disease-modulating cell state transitions, in accordance with an embodiment of the present disclosure.

FIGS. 10A and 10B illustrate prediction of clinical response to checkpoint inhibitors using characterized cellular constituent modules, in accordance with an embodiment of the present disclosure. FIGS. 10A and 10B illustrate conserved upregulation of the TCF7 module (15) and downregulation of the PDCD1 module (6) and the LAG3+TIGIT module (26) between in vitro Compound K response data and in vivo public patient data.

FIG. 11 illustrates a genome-wide association study (GWAS) integration approach for prediction of association of megakaryocytes with platelet diseases, in accordance with an embodiment of the present disclosure.

FIG. 12 illustrates a heatmap of integrated copy number variation (CNV) profiles and scRNA-seq DCX+ cells for the determination of malignant cancer cells, in accordance with an embodiment of the present disclosure. CNVs for each cell were clustered and each cell (row) was annotated by clusters inferring malignancy and DCX+ state.

FIG. 13 illustrates prediction of intercellular communication in a triple negative breast cancer (TNBC) tumor microenvironment, in accordance with an embodiment of the present disclosure.

FIG. 14A illustrates an experimental scheme of culturing the undifferentiated basal cells known as primary human bronchial epithelial cells (HBECs) at the air-liquid interface in the presence or absence of IL-13 and the perturbagens. FIG. 14B illustrates the testing results for Air-liquid interface (ALI) differentiation of HBECs in the presence or absence of IL-3 and shows that IL-13 treatment during ALI differentiation induces goblet cell hyperplasia and reduces ciliated cells. Pseudostratified airway epithelium was formed by day 14 of ALI differentiation in the presence of 1 ng/mL of IL-3. Total mucus (AB/PAS), goblet cells (Muc5ac⁺), and ciliated cells (acetyl α-tubulin⁺) were visualized via immunofluorescence or histology. For histology analysis, the differentiated HBECs on day 14 of ALI were stained with AB/PAS, Muc5ac, and acetyl α-tubulin. For Immunofluorescence imaging, the differentiated HBECs on day 14 of ALI were stained with DAPI, Muc5ac, and acetyl α-tubulin. Immunofluorescence allows for a top down view of the most apical layer of cells, while histology presents a cross-section of the pseudostratified epithelium and is amenable for image quantification.

FIG. 15A illustrates results from a qPCR assay demonstrating that Compound B1 predictably inhibits goblet cells and increases club cells. Using scRNA-seq data, Compound B1 was predicted to inhibit goblet cells and promote club cells within the top 1.2% of compounds of the intervention library. HBECs from healthy donors were differentiated at air-liquid interface (ALI) in the presence or absence of 0.3 ng/ml IL-13 and treated with predicted compounds. On day 14, cultures were lysed, RNA extracted, and cDNA generated. Goblet and club cells were assessed by Muc5ac and Scgblal primers, respectively. FIG. 15B illustrates results from a qPCR assay demonstrating that Compound B2 predictably inhibits goblet cells and increases ciliated cells. Using scRNA-seq data, Compound B2 was predicted to inhibit goblet cells and promote ciliated cells within the top 1.2% of compounds of the intervention library. HBECs from healthy donors were differentiated at air-liquid interface (ALI) in the presence or absence of 0.3 ng/ml IL-13 and treated with predicted compounds. On day 14, cultures were lysed, RNA extracted, and cDNA generated. Goblet and ciliated cells were assessed by Muc5ac and Foxj1 primers, respectively. FIG. 15C illustrates results from an immunofluorescence assay demonstrating that a Compound B3 predictably decreased goblet cells and increased ciliated cells. Using scRNA-seq data, Compound B3 was predicted to inhibit goblet cells and promote ciliated cells within the top 1.2% of compounds of the intervention library. HBECs from healthy donors were differentiated at air-liquid interface (ALI) in the presence or absence of 0.3 ng/ml IL-13 and treated with predicted compounds. On day 14, samples were fixed, processed for histology, anti-Muc5ac (goblet cells) and anti-acetyl α-Tubulin (ciliated cells) antibodies.

FIGS. 16A and 16B illustrates simulated (16A) versus measured (16B) impact of chaetocin in blood cells in accordance with an embodiment of the present disclosure.

### DETAILED DESCRIPTION

### Introduction

Given the above background, the present disclosure describes an approach to drug discovery that targets cellular processes and programs that are critical to disease. This approach is realized, in some aspects, by predicting therapeutic modalities and their properties through tying them to computationally engineered representations of cellular programs (e.g., cellular processes).

For example, in some aspects, the present disclosure provides algorithms to learn disease-critical cellular processes from scRNA-seq and related data types (e.g., cellular constituents).

In some embodiments, a computational modeling architecture with predictive capabilities is used to discover these disease-driving cell behaviors, through the generation of digital representations (e.g., latent representations) of disease-associated features in molecular data across one or more domains and/or data types. For instance, in some implementations, the method combines and determines correlations between a variety of data types (phenotypic, transcriptional, genetic, epigenetic, and/or covariate data) using latent representations and machine learning to predict disease-critical cellular processes.

In some aspects, these algorithms can be further paired with enrichments and annotations by other data modalities, such as genome-wide association studies (GWAS) and gene set enrichment analysis (GSEA).

In an example embodiment, the present disclosure provides a sparse-autoencoder-based modeling approach for learning and predicting disease-critical features from patient samples and pre-clinical disease models (e.g., in vivo and/or in vitro), where features that typically represent disease-critical cellular programs are targeted and clustered. The model is trained jointly on paired phenotypic-transcriptional data and data from a knowledge domain (e.g., GWAS, GSEA, annotations, etc.). In some instances, the model is additionally trained on genetic and epigenetic data. Targeted features are grouped into “modules” (e.g., disease modules, gene modules, cellular constituent modules, etc.), allowing complex molecular and phenotypic disease variations to be decomposed into targetable, actionable units.

Advantageously, the systems and methods disclosed herein address the shortcomings described above by providing systematic, scalable discovery of molecular targets for drug discovery. For instance, by virtue of the abovementioned modules being proxies for cellular programs that can be activated in a variety of cell types and cell states, targeting modules allows systematic targeting of a multitude of cell behaviors. Additionally, decomposing complex cellular constituent (e.g., transcriptional) and multi-readout cell representations into modules removes the well-known sensitivity of deep learning to details of system context, which alleviates the need of data-driven domain adaptation techniques, allowing the overall model architecture to be more interpretable. Furthermore, modules are interpretable and meaningfully associate with different stages of cellular biological processes and pathways. These interpretations facilitate downstream decision-making and application by researchers and scientists, thus contributing to the prioritization of relevant programs.

For example, as illustrated in Examples 1 and 2 below, identification of cellular constituent modules can be used to determine module activation in, and thus association with, a variety of cellular behaviors and phenotypes (e.g., cell lineages, cell state transitions, and/or responses to compound treatment). Furthermore, as illustrated in Examples 3 and 4 below, comparison of module activation with the knowledge domain (e.g., previous observations and/or annotations obtained from patient data, GWAS, and/or GSEA) can be used to determine relevant modules for further validation, improving the efficiency and applicability of the method to drug discovery by reducing the pool of candidate targets for validation to those with the greatest likelihood of efficacy. Similarly, as described in Examples 5 and 6, additional data types can be incorporated into the method, such as genomic data (e.g., copy number variations), which provide additional biological interpretations and actionable inferences such as malignancy, intercellular interactions and role in disease (e.g., cancers) leading to the identification of critical cell targets for downstream application.

Advantageously, the present disclosure further provides various systems and methods that improve the associating a plurality of cellular constituents with a cellular process of interest, by improving the training and use of a model for targeted determination of correlations between cellular constituent modules and covariates. The complexity of a machine learning model includes time complexity (running time, or the measure of the speed of an algorithm for a given input size n), space complexity (space requirements, or the amount of computing power or memory needed to execute an algorithm for a given input size n), or both. Complexity (and subsequent computational burden) applies to both training of and prediction by a given model.

In some instances, computational complexity is impacted by implementation, incorporation of additional algorithms or cross-validation methods, and/or one or more parameters (e.g., weights and/or hyperparameters). In some instances, computational complexity is expressed as a function of input size n, where input data is the number of instances (e.g., the number of training samples), dimensions p (e.g., the number of features), the number of trees nₜᵣₑₑₛ (e.g., for methods based on trees), the number of support vectors nₛᵥ (e.g., for methods based on support vectors), the number of neighbors k (e.g., for k nearest neighbor algorithms), the number of classes c, and/or the number of neurons nᵢ at a layer i (e.g., for neural networks). With respect to input size n, then, an approximation of computational complexity (e.g., in Big O notation) denotes how running time and/or space requirements increase as input size increases. Functions can increase in complexity at slower or faster rates relative to an increase in input size. Various approximations of computational complexity include but are not limited to constant (e.g., O(1)), logarithmic (e.g., O(log n)), linear (e.g., O(n)), loglinear (e.g., O(n log n)), quadratic (e.g., O(n²)), polynomial (e.g., O(n^{c}), exponential (e.g., O(cⁿ)), and/or factorial (e.g., O(n!)). In some instances, simpler functions are accompanied by lower levels of computational complexity as input sizes increase, as in the case of constant functions, whereas more complex functions such as factorial functions can exhibit substantial increases in complexity in response to slight increases in input size.

Computational complexity of machine learning models can similarly be represented by functions (e.g., in Big O notation), and complexity may vary depending on the type of model, the size of one or more inputs or dimensions, usage (e.g., training and/or prediction), and/or whether time or space complexity is being assessed. For example, complexity in decision tree algorithms is approximated as O(n²p) for training and O(p) for predictions, while complexity in linear regression algorithms is approximated as O(p²n+p³) for training and O(p) for predictions. For random forest algorithms, training complexity is approximated as O(n²pnₜᵣₑₑₛ) and prediction complexity is approximated as O(pnₜᵣₑₑₛ). For gradient boosting algorithms, complexity is approximated as O(npnₜᵣₑₑₛ) for training and O(pnₜᵣₑₑₛ) for predictions. For kernel support vector machines, complexity is approximated as O(n² p+n³) for training and O(nₛᵥp) for predictions. For naïve Bayes algorithms, complexity is represented as O(np) for training and O(p) for predictions, and for neural networks, complexity is approximated as O(pn₁+n₁n₂+ . . . ) for predictions. Complexity in K nearest neighbors algorithms is approximated as O(knp) for time and O(np) for space. For logistic regression algorithms, complexity is approximated as O(np) for time and O(p) for space. For logistic regression algorithms, complexity is approximated as O(np) for time and O(p) for space.

As described above, for machine learning models, computational complexity determines the scalability and therefore the overall effectiveness and usability of a model (e.g., a classifier) for increasing input, feature, and/or class sizes, as well as for variations in model architecture. In the context of large-scale datasets, as in the case of gene expression datasets comprising abundances of at least 10, at least 100, at least 1000 or more genes obtained for at least 10, at least 100, at least 1000 or more cells, the computational complexity of functions performed on such large datasets may strain the capabilities of many existing systems. In addition, as the number of input features (e.g., number of cellular constituents (e.g., genes)) and/or the number of instances (e.g., number of cells, cell state annotations, and/or covariates) increases together with technological advancements, increasing availability of annotations, and expanding downstream applications and possibilities, the computational complexity of any given classification model can quickly overwhelm the time and space capacities provided by the specifications of a respective system.

Thus, by using a machine learning model with a minimum input size (e.g., at least 10, at least 20, at least 100 or more cells; at least 10, at least 50, at least 100 or more cellular constituents; and/or at least 5, at least 10, at least 50 or more cellular constituent modules) and/or a corresponding minimum number of parameters (e.g., corresponding to every possible pairing of all of the features input to the machine learning model) for the associating a plurality of cellular constituents with a cellular process of interest, the computational complexity is proportionally increased such that it cannot be mentally performed, and the method addresses a computational problem. For example, in an embodiment of the present disclosure, obtaining a latent representation dimensioned by a plurality of at least 10 cellular constituent modules and a plurality of at least 50 cellular constituents or a representation thereof comprises obtaining at least 500 parameters (e.g., weights). In another embodiment of the present disclosure, obtaining a respective activation weight for each cellular constituent module in a plurality of at least 10 cellular constituent modules, for each cell in a plurality of at least 20 cells comprises obtaining at least 200 activation weights. Imposing similar minimums for additional input features and/or instances, including but not limited to number of cell state annotations, covariates, samples, time points, replicates, and/or batches, will similarly affect the computational complexity of the method.

Additional details on computational complexity in machine learning models are provided in “Computational complexity of machine learning algorithms,” published Apr. 16, 2018, available online at: thekerneltrip.com/machine/learning/computational-complexity-learning-algorithms; Hastie, 2001, The Elements of Statistical Learning, Springer, New York; and Arora and Barak, 2009, Computational Complexity: A Modern Approach, Cambridge University Press, New York; each of which is hereby incorporated herein by reference in its entirety.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

Plural instances may be provided for components, operations or structures described herein as a single instance. Finally, boundaries between various components, operations, and data stores are somewhat arbitrary, and particular operations are illustrated in the context of specific illustrative configurations. Other forms of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms “first,” “second,” etc. may be used herein to describe various elements, these elements should not be limited by these terms. These terms are only used to distinguish one element from another. For example, a first dataset could be termed a second dataset, and, similarly, a second dataset could be termed a first dataset, without departing from the scope of the present invention. The first dataset and the second dataset are both datasets, but they are not the same dataset.

The terminology used herein is for the purpose of describing particular implementations only and is not intended to be limiting of the claims. As used in the description of the implementations and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined (that a stated condition precedent is true)” or “if (a stated condition precedent is true)” or “when (a stated condition precedent is true)” may be construed to mean “upon determining” or “in response to determining” or “in accordance with a determination” or “upon detecting” or “in response to detecting” that the stated condition precedent is true, depending on the context.

Furthermore, when a reference number is given an “i^{th}” denotation, the reference number refers to a generic component, set, or embodiment. For instance, a cellular-component termed “cellular-component i” refers to the i^{th} cellular-component in a plurality of cellular-components.

The foregoing description included example systems, methods, techniques, instruction sequences, and computing machine program products that embody illustrative implementations. For purposes of explanation, numerous specific details are set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions below are not intended to be exhaustive or to limit the implementations to the precise forms disclosed. Many modifications and variations are possible in view of the above teachings. The implementations are chosen and described in order to best explain the principles and their practical applications, to thereby enable others skilled in the art to best utilize the implementations and various implementations with various modifications as are suited to the particular use contemplated.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention.

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

### Definitions

As used herein, the term “about” or “approximately” means within an acceptable error range for the particular value as determined by one of ordinary skill in the art, which depends in part on how the value is measured or determined, e.g., the limitations of the measurement system. For example, in some embodiments “about” means within 1 or more than 1 standard deviation, per the practice in the art. In some embodiments, “about” means a range of ±20%, ±10%, ±5%, or ±1% of a given value. In some embodiments, the term “about” or “approximately” means within an order of magnitude, within 5-fold, or within 2-fold, of a value. Where particular values are described in the application and claims, unless otherwise stated the term “about” meaning within an acceptable error range for the particular value can be assumed. All numerical values within the detailed description herein are modified by “about” the indicated value, and consider experimental error and variations that would be expected by a person having ordinary skill in the art. The term “about” can have the meaning as commonly understood by one of ordinary skill in the art. In some embodiments, the term “about” refers to ±10%. In some embodiments, the term “about” refers to ±5%.

As used herein, the terms “abundance,” “abundance level,” or “expression level” refers to an amount of a cellular constituent (e.g., a gene product such as an RNA species, e.g., mRNA or miRNA, or a protein molecule) present in one or more cells, or an average amount of a cellular constituent present across multiple cells. When referring to mRNA or protein expression, the term generally refers to the amount of any RNA or protein species corresponding to a particular genomic locus, e.g., a particular gene. However, in some embodiments, an abundance can refer to the amount of a particular isoform of an mRNA or protein corresponding to a particular gene that gives rise to multiple mRNA or protein isoforms. The genomic locus can be identified using a gene name, a chromosomal location, or any other genetic mapping metric.

As used interchangeably herein, a “cell state” or “biological state” refers to a state or phenotype of a cell or a population of cells. For example, a cell state can be healthy or diseased. A cell state can be one of a plurality of diseases. A cell state can be a response to a compound treatment and/or a differentiated cell lineage. A cell state can be characterized by a measure of one or more cellular constituents, including but not limited to one or more genes, one or more proteins, and/or one or more biological pathways.

As used herein, a “cell state transition” or “cellular transition” refers to a transition in a cell's state from a first cell state to an altered cell state (e.g., healthy to diseased). A cell state transition can be marked by a change in cellular constituent abundance in the cell, and thus by the identity and quantity cellular constituents (e.g., mRNA, transcription factors) produced by the cell.

As used herein, the term “dataset” in reference to cellular constituent abundance measurements for a cell or a plurality of cells can refer to a high-dimensional set of data collected from a single cell (e.g., a single-cell cellular constituent abundance dataset) in some contexts. In other contexts, the term “dataset” can refer to a plurality of high-dimensional sets of data collected from single cells (e.g., a plurality of single-cell cellular constituent abundance datasets), each set of data of the plurality collected from one cell of a plurality of cells.

As used herein, the term “differential abundance” or “differential expression” refers to differences in the quantity and/or the frequency of a cellular constituent present in a first entity (e.g., a first cell, plurality of cells, and/or sample) as compared to a second entity (e.g., a second cell, plurality of cells, and/or sample). In some embodiments, a first entity is a sample characterized by a first cell state (e.g., a diseased phenotype) and a second entity is a sample characterized by a second cell state (e.g., a normal or healthy phenotype). For example, a cellular constituent can be a polynucleotide (e.g., an mRNA transcript) which is present at an elevated level or at a decreased level in entities characterized by a first cell state compared to entities characterized by a second cell state. In some embodiments, a cellular constituent can be a polynucleotide which is detected at a higher frequency or at a lower frequency in entities characterized by a first cell state compared to entities characterized by a second cell state. A cellular constituent can be differentially abundant in terms of quantity, frequency or both. In some instances, a cellular constituent is differentially abundant between two entities if the amount of the cellular constituent in one entity is statistically significantly different from the amount of the cellular constituent in the other entity. For example, a cellular constituent is differentially abundant in two entities if it is present at least about 120%, at least about 130%, at least about 150%, at least about 180%, at least about 200%, at least about 300%, at least about 500%, at least about 700%, at least about 900%, or at least about 1000% greater in one entity than it is present in the other entity, or if it is detectable in one entity and not detectable in the other. In some instances, a cellular constituent is differentially expressed in two sets of entities if the frequency of detecting the cellular constituent in a first subset of entities (e.g., cells representing a first subset of annotated cell states) is statistically significantly higher or lower than in a second subset of entities (e.g., cells representing a second subset of annotated cell states). For example, a cellular constituent is differentially expressed in two sets of entities if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of entities than the other set of entities.

As used herein, the term “perturbation” in reference to a cell (e.g., a perturbation of a cell or a cellular perturbation) refers to any treatment of the cell with one or more compounds. These compounds can be referred to as “perturbagens.” In some embodiments, the perturbagen can include, e.g., a small molecule, a biologic, a therapeutic, a protein, a protein combined with a small molecule, an ADC, a nucleic acid, such as an siRNA or interfering RNA, a cDNA over-expressing wild-type and/or mutant shRNA, a cDNA over-expressing wild-type and/or mutant guide RNA (e.g., Cas9 system or other gene editing system), or any combination of any of the foregoing.

As used herein, the term “sample,” “biological sample,” or “patient sample,” refers to any sample taken from a subject, which can reflect a biological state associated with the subject. Examples of samples include, but are not limited to, blood, whole blood, plasma, serum, urine, cerebrospinal fluid, fecal, saliva, sweat, tears, pleural fluid, pericardial fluid, or peritoneal fluid of the subject. A sample can include any tissue or material derived from a living or dead subject. A sample can be a cell-free sample. A sample can comprise one or more cellular constituents. For instance, a sample can comprise a nucleic acid (e.g., DNA or RNA) or a fragment thereof, or a protein. The term “nucleic acid” can refer to deoxyribonucleic acid (DNA), ribonucleic acid (RNA) or any hybrid or fragment thereof. The nucleic acid in the sample can be a cell-free nucleic acid. A sample can be a liquid sample or a solid sample (e.g., a cell or tissue sample). A sample can be a bodily fluid. A sample can be a stool sample. A sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis.

As used herein the term “fingerprint” as in a fingerprint of a compound is a digital digest of the compound. Nonlimiting examples of such a digital digest include Daylight fingerprints, a BCI fingerprint, an ECFC4 fingerprint, an ECFP4 fingerprint, an EcFC fingerprint, an MDL fingerprint, an atom pair fingerprint (APFP fingerprint), a topological torsion fingerprint (TTFP) fingerprint, a UNITY 2D fingerprint, an RNNS2S fingerprint, or a GraphConv fingerprint. See Franco, 2014, “The Use of 2D fingerprint methods to support the assessment of structural similarity in orphan drug legislation,” J. Cheminform 6, p. 5, and Rensi and Altman, 2017, “Flexible Analog Search with Kernel PCA Embedded Molecule Vectors,” Computational and Structural Biotechnology Journal, doi:10.1016/j.csbj.2017.03.003, each of which is hereby incorporated by reference. See also Raymond and Willett, 2002, “Effectiveness of graph-based and fingerprint-based similarity measures for virtual screening of 2D chemical structure databases,” Journal of Computer-Aided Molecular Design 16, 59-71, and Franco et al., 2014, “The use of 2D fingerprint methods to support the assessment of structural similarity in orphan drug legislation” Journal of chemoinformatics 6(5), each of which is hereby incorporated by reference.

As used herein the term “classification” can refer to any number(s) or other characters(s) that are associated with a particular property (e.g., a cellular process, a covariate, a cell state annotation, etc.) of an entity (e.g., a cell, a sample, a cellular constituent, a cellular constituent modules, etc.). For example, a “+” symbol (or the word “positive”) can signify that an entity is classified as positive for a particular property (e.g., a cellular constituent module is positively associated with a cellular process of interest). In another example, the term “classification” can refer to a determination of correlation between an entity and a particular property (e.g., a correlation between a respective covariate and a respective cellular constituent module). In some embodiments, the classification is a correlation coefficient and/or a weight. The classification can be binary (e.g., positive or negative) or have more levels of classification (e.g., a scale from 1 to 10 or 0 to 1). The terms “cutoff” and “threshold” can refer to predetermined numbers used in an operation. For example, a cutoff value can refer to a value above which entities are excluded. A threshold value can be a value above or below which a particular classification applies. Either of these terms can be used in either of these contexts.

As used interchangeably herein, the term “classifier”, “model”, algorithm, “regressor”, and/“or classifier” refers to a machine learning model or algorithm. In some embodiments, a model is an unsupervised learning algorithm. One example of an unsupervised learning algorithm is cluster analysis.

In some embodiments, a model is supervised machine learning. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, GradientBoosting, mixture models, hidden Markov models, Gaussian NB algorithms, linear discriminant analysis, or any combinations thereof. In some embodiments, a model is a multinomial classifier algorithm. In some embodiments, a model is a 2-stage stochastic gradient descent (SGD) model. In some embodiments, a model is a deep neural network (e.g., a deep-and-wide sample-level model). In some embodiments, a classifier or model of the present disclosure has 25 or more, 100 or more, 1000 or more 10,000 or more, 100,000 or more or 1×10⁶ or more parameters and thus the calculations of the model cannot be mentally be performed.

Moreover, as used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. As such, the algorithms, models, regressors, and/or classifiers of the present disclosure cannot be mentally performed. In some embodiments, n is between 10,000 and 1×10⁷, between 100,000 and 5×10⁶, or between 500,000 and 1×10⁶. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

Neural networks. In some embodiments, the model is a neural network (e.g., a convolutional neural network and/or a residual neural network). Neural network algorithms, also known as artificial neural networks (ANNs), include convolutional and/or residual neural network algorithms (deep learning algorithms). Neural networks can be machine learning algorithms that may be trained to map an input data set to an output data set, where the neural network comprises an interconnected group of nodes organized into multiple layers of nodes. For example, the neural network architecture may comprise at least an input layer, one or more hidden layers, and an output layer. The neural network may comprise any total number of layers, and any number of hidden layers, where the hidden layers function as trainable feature extractors that allow mapping of a set of input data to an output value or set of output values. As used herein, a deep learning algorithm (DNN) can be a neural network comprising a plurality of hidden layers, e.g., two or more hidden layers. Each layer of the neural network can comprise a number of nodes (or “neurons”). A node can receive input that comes either directly from the input data or the output of nodes in previous layers, and perform a specific operation, e.g., a summation operation. In some embodiments, a connection from an input to a node is associated with a parameter (e.g., a weight and/or weighting factor). In some embodiments, the node may sum up the products of all pairs of inputs, xi, and their associated parameters. In some embodiments, the weighted sum is offset with a bias, b. In some embodiments, the output of a node or neuron may be gated using a threshold or activation function, f, which may be a linear or non-linear function. The activation function may be, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training data set and a gradient descent or backward propagation method so that the output value(s) that the ANN computes are consistent with the examples included in the training data set. The parameters may be obtained from a back propagation neural network training process.

Any of a variety of neural networks may be suitable for use in analyzing an image of a subject. Examples can include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. Convolutional and/or residual neural networks can be used for analyzing an image of a subject in accordance with the present disclosure.

For instance, a deep neural network model comprises an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network model. In some embodiments, at least 100 parameters, at least 1000 parameters, at least 2000 parameters or at least 5000 parameters are associated with the deep neural network model. As such, deep neural network models require a computer to be used because they cannot be mentally solved. In other words, given an input to the model, the model output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, Mass., USA: MIT Press, each of which is hereby incorporated by reference.

Neural network algorithms, including convolutional neural network algorithms, suitable for use as models are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as models are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as models are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, N.Y., each of which is hereby incorporated by reference in its entirety.

Support vector machines. In some embodiments, the model is a support vector machine (SVM). SVM algorithms suitable for use as models are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space can correspond to a non-linear decision boundary in the input space. In some embodiments, the plurality of parameters (e.g., weights) associated with the SVM define the hyper-plane. In some embodiments, the hyper-plane is defined by at least 10, at least 20, at least 50, or at least 100 parameters and the SVM model requires a computer to calculate because it cannot be mentally solved.

Naïve Bayes algorithms. In some embodiments, the model is a Naive Bayes algorithm. Naïve Bayes models suitable for use as models are disclosed, for example, in Ng et al., 2002, “On discriminative vs. generative classifiers: A comparison of logistic regression and naive Bayes,” Advances in Neural Information Processing Systems, 14, which is hereby incorporated by reference. A Naive Bayes model is any model in a family of “probabilistic models” based on applying Bayes' theorem with strong (naïve) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference.

Nearest neighbor algorithms. In some embodiments, a model is a nearest neighbor algorithm. Nearest neighbor models can be memory-based and include no model to be fit. For nearest neighbors, given a query point xo (a test subject), the k training points xo, r, . . . , k (here the training subjects) closest in distance to xo are identified and then the point xo is classified using the k nearest neighbors. Here, the distance to these neighbors is a function of the abundance values of the discriminating gene set. In some embodiments, Euclidean distance in feature space is used to determine distance as d₍ᵢ₎=∥x₍ᵢ₎−x_{(O)}∥. Typically, when the nearest neighbor algorithm is used, the abundance data used to compute the linear discriminant is standardized to have mean zero and variance 1. The nearest neighbor rule can be refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

A k-nearest neighbor model is a non-parametric machine learning method in which the input consists of the k closest training examples in feature space. The output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See, Duda et al., 2001, Pattern Classification, Second Edition, John Wiley & Sons, which is hereby incorporated by reference. In some embodiments, the number of distance calculations needed to solve the k-nearest neighbor model is such that a computer is used to solve the model for a given input because it cannot be mentally performed.

Random forest, decision tree, and boosted tree algorithms. In some embodiments, the model is a decision tree. Decision trees suitable for use as models are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety. In some embodiments, the decision tree model includes at least 10, at least 20, at least 50, or at least 100 parameters (e.g., weights and/or decisions) and requires a computer to calculate because it cannot be mentally solved.

Regression. In some embodiments, the model uses a regression algorithm and thus can be referred to as a regressor. A regressor can use any type of regression. For example, in some embodiments, the regression algorithm is logistic regression. In some embodiments, the regression algorithm is logistic regression with lasso, L2 or elastic net regularization. In some embodiments, those extracted features that have a corresponding regression coefficient that fails to satisfy a threshold value are pruned (removed from) consideration. In some embodiments, a generalization of the logistic regression model that handles multicategory responses is used as the model. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the regressor makes use of a regression model disclosed in Hastie et al., 2001, The Elements of Statistical Learning, Springer-Verlag, New York. In some embodiments, the logistic regression includes at least 10, at least 20, at least 50, at least 100, or at least 1000 parameters (e.g., weights) and requires a computer to calculate because it cannot be mentally solved.

Linear discriminant analysis algorithms. Linear discriminant analysis (LDA), normal discriminant analysis (NDA), or discriminant function analysis can be a generalization of Fisher's linear discriminant, a method used in statistics, pattern recognition, and machine learning to find a linear combination of features that characterizes or separates two or more classes of objects or events. The resulting combination can be used as the model (linear model) in some embodiments of the present disclosure.

Mixture model and Hidden Markov model. In some embodiments, the model is a mixture model, such as that described in McLachlan et al., Bioinformatics 18(3):413-422, 2002. In some embodiments, in particular, those embodiments including a temporal component, the model is a hidden Markov model such as described by Schliep et al., 2003, Bioinformatics 19(1):i255-i263.

Clustering. In some embodiments, the model is an unsupervised clustering model. In some embodiments, the model is a supervised clustering model. Clustering algorithms suitable for use as models are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. The clustering problem can be described as one of finding natural groupings in a dataset. To identify natural groupings, two issues can be addressed. First, a way to measure similarity (or dissimilarity) between two samples can be determined. This metric (e.g., similarity measure) can be used to ensure that the samples in one cluster are more like one another than they are to samples in other clusters. Second, a mechanism for partitioning the data into clusters using the similarity measure can be determined. One way to begin a clustering investigation can be to define a distance function and to compute the matrix of distances between all pairs of samples in the training set. If distance is a good measure of similarity, then the distance between reference entities in the same cluster can be significantly less than the distance between the reference entities in different clusters. However, clustering may not use of a distance metric. For example, a nonmetric similarity function s(x, x′) can be used to compare two vectors x and x′. s(x, x′) can be a symmetric function whose value is large when x and x′ are somehow “similar.” Once a method for measuring “similarity” or “dissimilarity” between points in a dataset has been selected, clustering can use a criterion function that measures the clustering quality of any partition of the data. Partitions of the data set that extremize the criterion function can be used to cluster the data. Particular exemplary clustering techniques that can be used in the present disclosure can include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering (e.g., with no preconceived number of clusters and/or no predetermination of cluster assignments).

Ensembles of models and boosting. In some embodiments, an ensemble (two or more) of models is used. In some embodiments, a boosting technique such as AdaBoost is used in conjunction with many other types of learning algorithms to improve the performance of the model. In this approach, the output of any of the models disclosed herein, or their equivalents, is combined into a weighted sum that represents the final output of the boosted model. In some embodiments, the plurality of outputs from the models is combined using any measure of central tendency known in the art, including but not limited to a mean, median, mode, a weighted mean, weighted median, weighted mode, etc. In some embodiments, the plurality of outputs is combined using a voting method. In some embodiments, a respective model in the ensemble of models is weighted or unweighted.

As used herein, the term “untrained model” (e.g., “untrained classifier” and/or “untrained autoencoder”) refers to a machine learning algorithm such as a model or an autoencoder that has not been trained on a training dataset. As used herein, the term “training a model” refers to the process of training an untrained or partially trained model. For instance, in some embodiments, training a model comprises obtaining a plurality of cellular constituent modules arranged in a latent representation and a cellular constituent count data structure discussed below. The plurality of cellular constituent modules arranged in a latent representation and the cellular constituent count data structure are combined to form an activation data structure that is applied as collective input to an untrained or partially trained model, in conjunction with the actual absence of present of each covariate in a plurality of covariates for the plurality of cellular constituent modules in the activation data structure, (hereinafter “primary training dataset”) to train the untrained or partially trained model on covariate-module correlation, thereby obtaining a trained model. Moreover, it will be appreciated that the term “untrained model” does not exclude the possibility that transfer learning techniques are used in such training of the untrained model. For instance, Fernandes et al., 2017, “Transfer Learning with Partial Observability Applied to Cervical Cancer Screening,” Pattern Recognition and Image Analysis: 8^{th} Iberian Conference Proceedings, 243-250, which is hereby incorporated by reference, provides non-limiting examples of such transfer learning. In instances where transfer learning is used, the untrained model described above is provided with additional data over and beyond that of the primary training dataset. That is, in non-limiting examples of transfer learning embodiments, the untrained model receives (i) the primary training dataset and (ii) additional data. Typically, this additional data is in the form of coefficients (e.g., regression coefficients) that were learned from another, auxiliary training dataset. Moreover, while a description of a single auxiliary training dataset has been disclosed, it will be appreciated that there is no limit on the number of auxiliary training datasets that may be used to complement the primary training dataset in training the untrained model in the present disclosure. For instance, in some embodiments, two or more auxiliary training datasets, three or more auxiliary training datasets, four or more auxiliary training datasets or five or more auxiliary training datasets are used to complement the primary training dataset through transfer learning, where each such auxiliary dataset is different than the primary training dataset. Any manner of transfer learning may be used in such embodiments. For instance, consider the case where there is a first auxiliary training dataset and a second auxiliary training dataset in addition to the primary training dataset. The coefficients learned from the first auxiliary training dataset (by application of a model such as regression to the first auxiliary training dataset) may be applied to the second auxiliary training dataset using transfer learning techniques (e.g., two-dimensional matrix multiplication), which in turn may result in a trained intermediate model whose coefficients are then applied to the primary training dataset and this, in conjunction with the primary training dataset itself, is applied to the untrained model. Alternatively, a first set of coefficients learned from the first auxiliary training dataset (by application of a model such as regression to the first auxiliary training dataset) and a second set of coefficients learned from the second auxiliary training dataset (by application of a model such as regression to the second auxiliary training dataset) may each individually be applied to a separate instance of the primary training dataset (e.g., by separate independent matrix multiplications) and both such applications of the coefficients to separate instances of the primary training dataset in conjunction with the primary training dataset itself (or some reduced form of the primary training dataset such as principal components or regression coefficients learned from the primary training set) may then be applied to the untrained model in order to train the untrained model. In either example, knowledge regarding covariate-module correlations (e.g., additional cell state annotations, additional covariates, and/or cellular constituent abundances thereof, etc.) derived from the first and second auxiliary training datasets is used, in conjunction with the covariate-labeled primary training dataset, to train the untrained model.

As used interchangeably herein, the term “neuron,” “node,” “unit,” “hidden neuron,” “hidden unit,” or the like, refers to a unit of a neural network that accepts input and provides an output via an activation function and one or more parameters (e.g., coefficients and/or weights). For example, a hidden neuron can accept one or more inputs from a prior layer and provide an output that serves as an input for a subsequent layer. In some embodiments, a neural network comprises only one output neuron. In some embodiments, a neural network comprises a plurality of output neurons. Generally, the output is a prediction value, such as a probability or likelihood, a binary determination (e.g., a presence or absence, a positive or negative result), and/or a label (e.g., a classification and/or a correlation coefficient) of a condition of interest such as a covariate, a cell state annotation, or a cellular process of interest. For single-class classification models, the output can be a likelihood (e.g., a correlation coefficient and/or a weight) of an input feature (e.g., one or more cellular constituent modules) having a condition (e.g., a covariate, a cell state annotation, and/or a cellular process of interest). For multi-class classification models, multiple prediction values can be generated, with each prediction value indicating the likelihood of an input feature for each condition of interest.

As used herein, the term “parameter” refers to any coefficient or, similarly, any value of an internal or external element (e.g., weight and/or hyperparameter) in a model, classifier, or algorithm that can affect (e.g., modify, tailor, and/or adjust) one or more inputs, outputs, and/or functions in the model, classifier, or algorithm. In some embodiments, parameters are coefficients (e.g., weights) that modulate one or more inputs, outputs, or functions in a classifier. For instance, a value of a parameter can be used to upweight or down-weight the influence of an input (e.g., a feature) to a classifier. Features can be associated with parameters, such as in a logistic regression, SVM, or naïve Bayes model. A value of a parameter can, alternately or additionally, be used to upweight or down-weight the influence of a node in a neural network (e.g., where the node comprises one or more activation functions that define the transformation of an input to an output), a class, or an instance (e.g., of a cell in a plurality of cells). Assignment of parameters to specific inputs, outputs, functions, or features is not limited to any one paradigm for a given classifier but can be used in any suitable classifier architecture for optimal performance. In some instances, reference to the parameters (e.g., coefficients) associated with the inputs, outputs, functions, or features of a classifier can similarly be used as an indicator of the number, performance, or optimization of the same, such as in the context of the computational complexity of machine learning algorithms. In some embodiments, a parameter has a fixed value. In some embodiments, a value of a parameter is manually and/or automatically adjustable (e.g., using a hyperparameter optimization method). In some embodiments, a value of a parameter is modified by a classifier validation and/or training process (e.g., by error minimization and/or backpropagation methods, as described elsewhere herein).

As used herein, the term “vector” is an enumerated list of elements, such as an array of elements, where each element has an assigned meaning. As such, the term “vector” as used in the present disclosure is interchangeable with the term “tensor.” As an example, if a vector comprises the abundance counts, in a plurality of cells, for a respective cellular constituent, there exists a predetermined element in the vector for each one of the plurality of cells. For ease of presentation, in some instances a vector may be described as being one-dimensional. However, the present disclosure is not so limited. A vector of any dimension may be used in the present disclosure provided that a description of what each element in the vector represents is defined (e.g., that element 1 represents abundance count of cell 1 of a plurality of cells, etc.).

As used herein, the term “cellular process” means a specific objective that a cell is genetically “programmed” to achieve. Each cellular process is often described by its outcome or ending state, e.g., the cellular process of cell division results in the creation of two daughter cells (a divided cell) from a single parent cell. A cellular process is accomplished by a particular set of molecular processes carried out by specific gene products, often in a highly regulated manner and in a particular temporal sequence. A determination that a particular cellular constituent is associated with a cellular process means that the cellular constituent carries out a molecular process that plays an integral role in that cellular process. But a cellular constituent can affect a biological objective even if it does not act strictly within the cellular process. First, a cellular constituent can control when and where the cellular process is executed; that is, it might regulate the cellular process. In this case, the cellular constituent acts outside of the cellular process, and controls (directly or indirectly) the activity of one or more cellular constituent that act within the cellular process. Second, the cellular constituent might act in another, separate cellular process that is required for the given cellular process to occur. For instance, animal embryogenesis requires translation, though translation would not generally be considered to be part of the embryogenesis program. Thus, a given cellular process could be associated with a cellular process by being an integral component of the cellular process, by regulating the cellular process, or be upstream of but still necessary for, the cellular process. Cellular processes span an entire range of how biologists characterize biological systems. They can be as simple as a generic enzymatic process, e.g., protein phosphorylation, to molecular pathways such as glycolysis or the canonical Wnt signaling pathway, to complex programs like embryo development or learning, and even including reproduction. Cellular processes also includes molecular-level processes that cannot always be distinguished from molecular functions. For instance, the cellular process class “protein phosphorylation” overlaps in meaning with the cellular process class “protein kinase activity,” as protein kinase activity is the enzymatic activity by which protein phosphorylation occurs. The main difference is that while a molecular function annotation has precise semantics (e.g., the gene carries out protein kinase activity), the cellular process annotation does not (e.g., the gene either carries out, regulates, or is upstream of but necessary for a particular protein kinase activity). Because of this diversity, in practice not all cellular process classes actually represent coherent, regulated biological programs. Cellular process, interchangeably referred to herein as “biological processes” are further described in The Gene Ontology Handbook, Methods in Molecular Biology, eds., Dessimoz and Skunca, 2017, Human Press, Springer Science+Business Media LLC New York, Chapter 2, which is hereby incorporated by reference.

### I. EXEMPLARY SYSTEM EMBODIMENTS

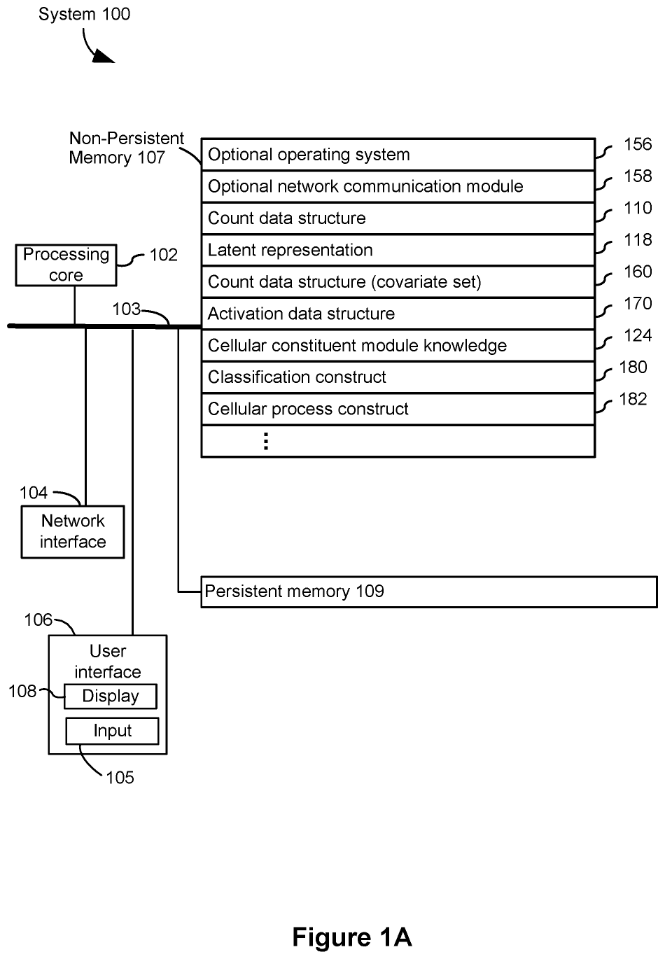

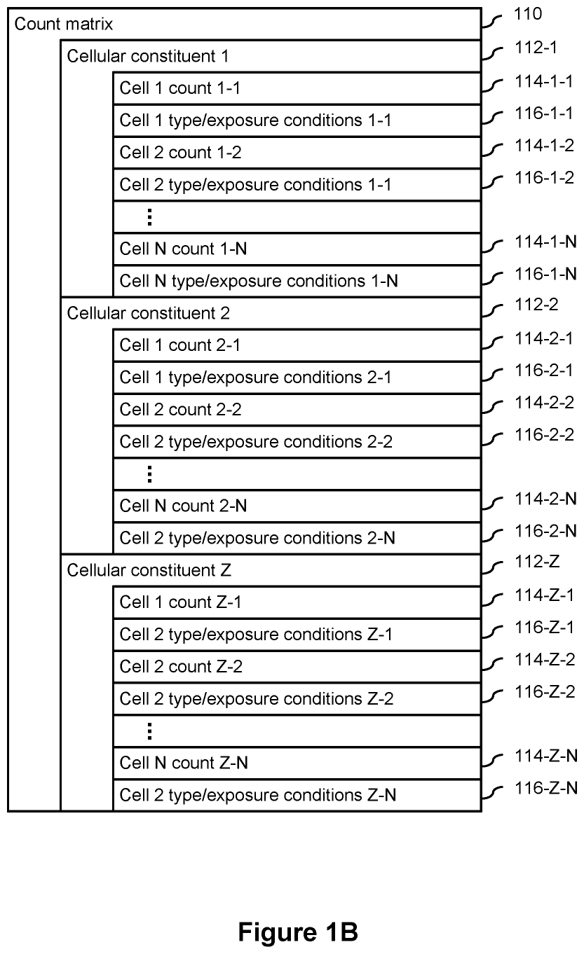

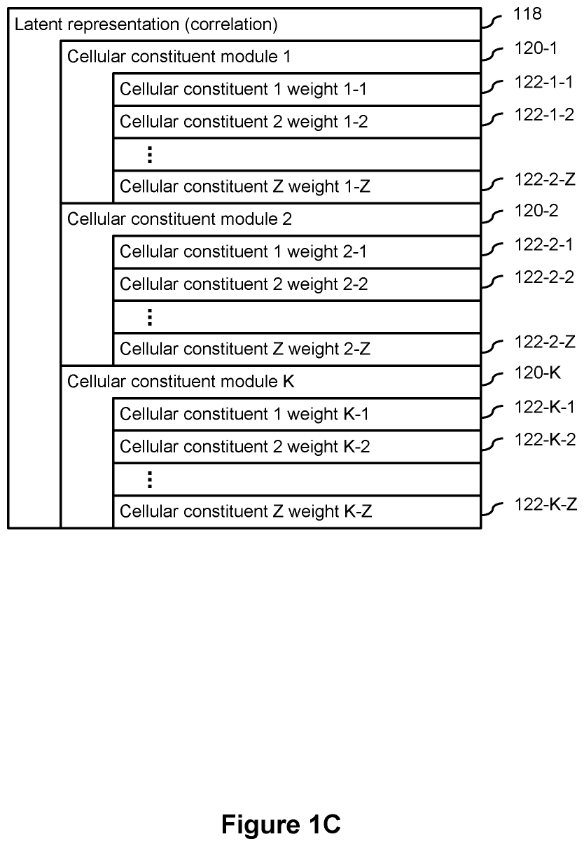

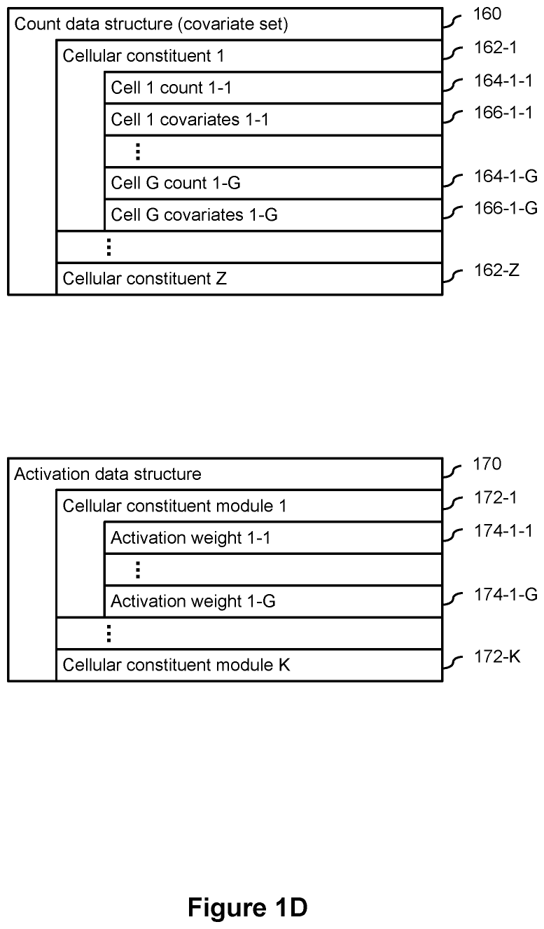

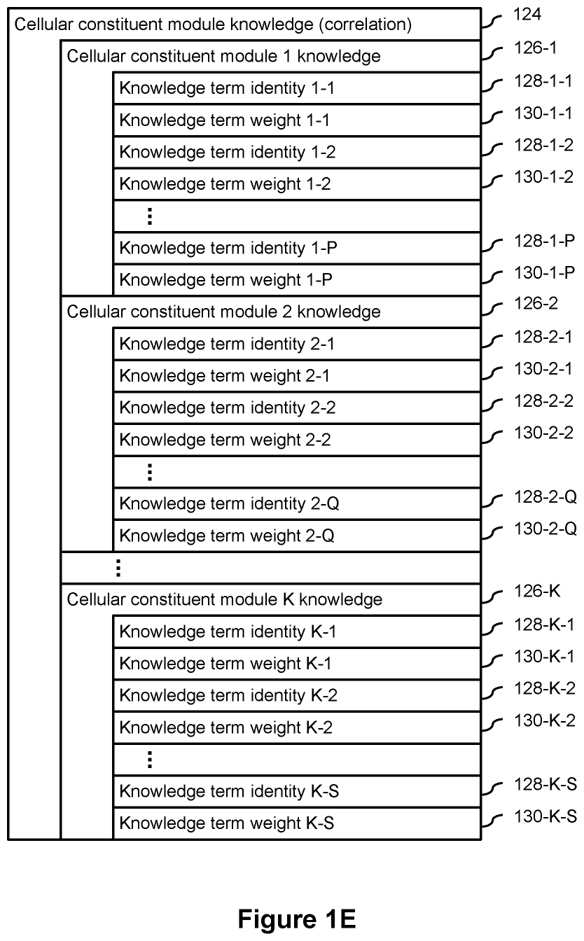

Now that an overview of some aspects of the present disclosure and some definitions used in the present disclosure have been provided, details of an exemplary system are described in conjunction with FIGS. 1A-E.

FIGS. 1A-E collectively provide a block diagram illustrating a system 100 in accordance with some embodiments of the present disclosure. The system 100 provides a determination of one or more cellular constituent modules in a plurality of cellular constituent modules that is associated with a cellular process of interest. In FIG. 1A, the system 100 is illustrated as a computing device. Other topologies of the computer system 100 are possible. For instance, in some embodiments, the system 100 can in fact constitute several computer systems that are linked together in a network, or be a virtual machine or a container in a cloud computing environment. As such, the exemplary topology shown in FIG. 1A merely serves to describe the features of an embodiment of the present disclosure in a manner that will be readily understood to one of skill in the art.

Referring to FIG. 1A, in some embodiments a computer system 100 (e.g., a computing device) includes a network interface 104. In some embodiments, the network interface 104 interconnects the system 100 computing devices within the system with each other, as well as optional external systems and devices, through one or more communication networks (e.g., through network communication module 158). In some embodiments, the network interface 104 optionally provides communication through network communication module 158 via the Internet, one or more local area networks (LANs), one or more wide area networks (WANs), other types of networks, or a combination of such networks.

Examples of networks include the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

The system 100 in some embodiments includes one or more processing units (CPU(s)) 102 (e.g., a processor, a processing core, etc.), one or more network interfaces 104, a user interface 106 including (optionally) a display 108 and an input system 105 (e.g., an input/output interface, a keyboard, a mouse, etc.) for use by the user, memory (e.g., non-persistent memory 107, persistent memory 109), and one or more communication buses 103 for interconnecting the aforementioned components. The one or more communication buses 103 optionally include circuitry (sometimes called a chipset) that interconnects and controls communications between system components. The non-persistent memory 107 typically includes high-speed random access memory, such as DRAM, SRAM, DDR RAM, ROM, EEPROM, flash memory, whereas the persistent memory 109 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 109 optionally includes one or more storage devices remotely located from the CPU(s) 102. The persistent memory 109, and the non-volatile memory device(s) within the non-persistent memory 109, include non-transitory computer readable storage medium. In some embodiments, the non-persistent memory 107 or alternatively the non-transitory computer readable storage medium stores the following programs, modules and data structures, or a subset thereof, sometimes in conjunction with the persistent memory 109:
an optional operating system 156 (e.g., ANDROID, iOS, DARWIN, RTXC, LINUX, UNIX, OS X, WINDOWS, or an embedded operating system such as VxWorks), which includes procedures for handling various basic system services and for performing hardware dependent tasks;
an optional network communication module (or instructions) 158 for connecting the system 100 with other devices and/or a communication network 104;
a count data structure for a first plurality of cells 110;
a latent representation 118;
a count data structure for a second plurality of cells 160;
an activation data structure 170;
a cellular constituent module knowledge store 124;
a classification construct 180; and
a cellular process construct 182.

In some embodiments, the count data structure for a first plurality of cells 110 comprises a count matrix comprising, for each cell in the first plurality of cells, a corresponding abundance for each cellular constituent in the plurality of cellular constituents. For example, as illustrated in FIG. 1B, the count matrix comprises, for each cell 114 in the first plurality of cells (e.g., 114-1-1, . . . 114-1-N), where the first plurality of cells collectively represents a plurality of annotated cell states 116 (e.g., cell types and/or exposure conditions 166-1-1, . . . , 166-1-N), for each cellular constituent 112 in a plurality of cellular constituents (e.g., 112-1, . . . 112-Z), a corresponding abundance 114 of the respective cellular constituent in the respective cell.

In some embodiments, the latent representation 118 is formed from the count data structure for the first plurality of cells 110 and represents correlations between each respective cellular constituent in the plurality of cellular constituents in the count data structure and each respective cellular constituent module in a plurality of cellular constituent modules. For example, as illustrated in FIG. 1C, the latent representation 118 includes, for each cellular constituent module 120 in a plurality of cellular constituent modules (e.g., 120-1, 120-2, 120-K), a weight 122 for each cellular constituent (e.g., gene) in the plurality of cellular constituents (e.g., a plurality of genes; 122-1-1, . . . 122-K-Z).

In some embodiments, the count data structure for a second plurality of cells 160 comprises a count data structure comprising, for each cell in the second plurality of cells, a corresponding abundance for each cellular constituent in the plurality of cellular constituents. For example, as illustrated in FIG. 1D, the count data structure includes, for each cell 164 in the second plurality of cells (e.g., 164-1-1, . . . 164-1-G), where the second plurality of cells collectively represents a plurality of covariates possibly informative of a cellular process of interest 166 (e.g., 166-1-1, . . . ,166-1-G), for each cellular constituent 162 in the plurality of cellular constituents (e.g., 162-1, . . . 162-Z), a corresponding abundance 164 of the respective cellular constituent in the respective cell.

In some embodiments, the activation data structure 170 is formed by combining the latent representation 118 and the count data structure for the second plurality of cells 160 using the plurality of cellular constituents as a common dimension. For example, as illustrated in FIG. 1D, the activation data structure 170 comprises, for each cellular constituent module 172 in the plurality of cellular constituent modules (e.g., 172-1, . . . 172-K), for each cell in the second plurality of cells, a respective activation weight 174 (e.g., 174-1-1, . . . 174-1-G).

In some embodiments, the cellular constituent module knowledge store 124 is obtained by training a model using the activation data structure 170 and represents correlations between each respective covariate in the plurality of covariates and each respective cellular constituent module in the plurality of cellular constituent modules. For example, as illustrated in FIG. 1E, the cellular constituent module knowledge store 124 includes, for each cellular constituent module 126 in a plurality of cellular constituent modules (e.g., 126-1, 126-2, 126-K), a knowledge term identity 128 in a plurality of knowledge term identities (e.g., 128-1-1, . . . 128-K-S) and a knowledge term weight 130 in a plurality of knowledge term weights (e.g., 130-1-1, . . . 130-K-S), where each knowledge term identity refers to a corresponding covariate possibly informative of a cellular process of interest and each knowledge term weight indicates whether the respective covariate correlates with the respective cellular constituent module.

In some embodiments, the classification construct 180 is used for training the model, where the training adjusts a plurality of covariate weights associated with the model responsive to a difference between a calculated and an actual activation against each cellular constituent model in the plurality of cellular constituent modules. In some embodiments, the cellular process construct 182 is used for identifying, using the plurality of covariate weights upon training the model, one or more cellular constituent modules in the plurality of cellular constituent modules that is associated with one or more covariates in the plurality of covariates.

In various embodiments, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the non-persistent memory 107 optionally stores a subset of the modules and data structures identified above. Furthermore, in some embodiments, the memory stores additional modules and data structures not described above. In some embodiments, one or more of the above identified elements is stored in a computer system, other than that of the system 100, that is addressable by the system 100 so that the system 100 may retrieve all or a portion of such data when needed.

Although FIGS. 1A, 1B, 1C, 1D, and 1E depict a “system 100,” the figure is intended more as a functional description of the various features that may be present in computer systems than as a structural schematic of the implementations described herein. In practice, and as recognized by those of ordinary skill in the art, items shown separately could be combined and some items could be separated. Moreover, although FIGS. 1A, 1B, 1C, 1D, and 1E depict certain data and modules in non-persistent memory 107, some or all of these data and modules instead may be stored in persistent memory 109 or in more than one memory. For example, in some embodiments, at least data store 160 and count data structure 110 are stored in a remote storage device which can be a part of a cloud-based infrastructure. In some embodiments, at least data store 160 and count data structure 110 are stored on a cloud-based infrastructure. In some embodiments, data store 160 and count data structure 110 can also be stored in the remote storage device(s).

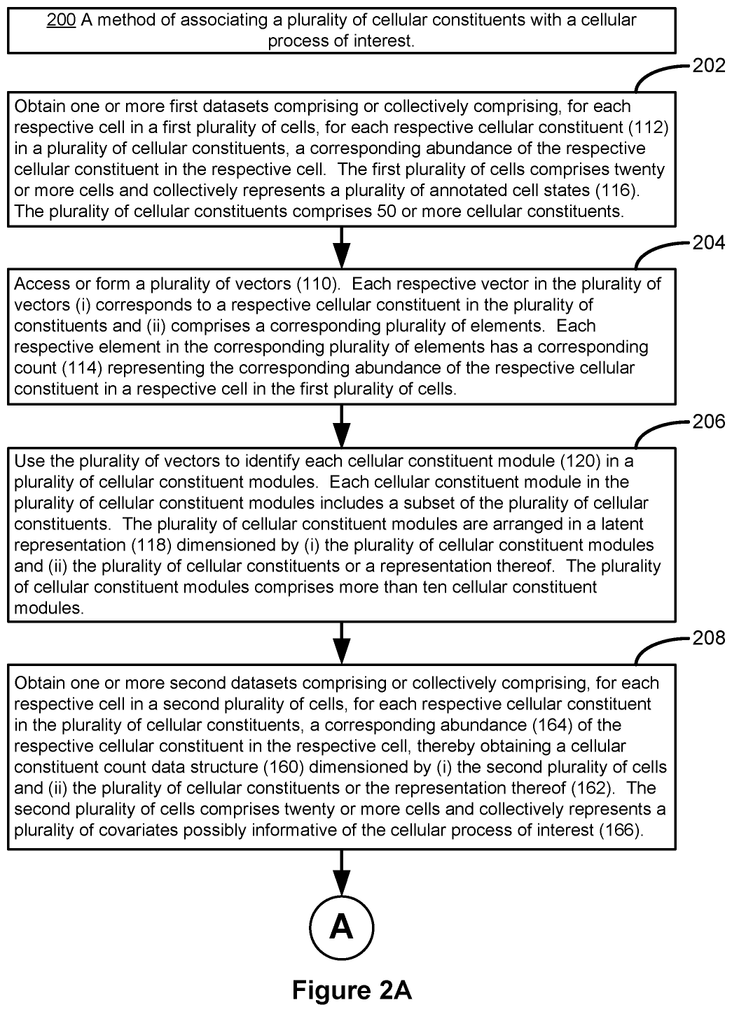

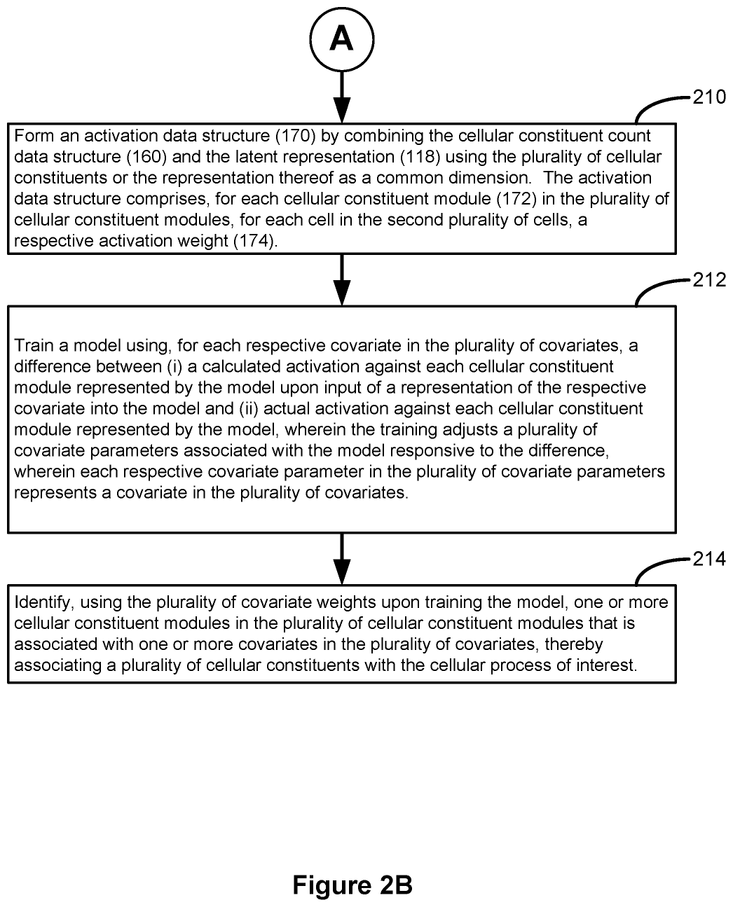

While a system in accordance with the present disclosure has been disclosed with reference to 1A, 1B, 1C, 1D, and 1E, methods 200 and 300 in accordance with the present disclosure are now detailed with reference to FIGS. 2A-B and 3.

### II. METHODS OF ASSOCIATING CELLULAR CONSTITUENTS WITH CELLULAR PROCESSES

Referring to FIGS. 2A and 2B, one aspect of the present disclosure provides a method 200 of associating a plurality of cellular constituents with a cellular process of interest.

Referring to Block 202, the method comprises obtaining one or more first datasets in electronic form, the one or more first datasets comprising or collectively comprising, for each respective cell in a first plurality of cells, where the first plurality of cells comprises, e.g., twenty or more cells and collectively represents a plurality of annotated cell states, for each respective cellular constituent in a plurality of cellular constituents, where the plurality of cellular constituents comprises, e.g., 50 or more cellular constituents, a corresponding abundance of the respective cellular constituent in the respective cell.

FIG. 1B illustrates an example dataset for the one or more first datasets, in accordance with some embodiments of the present disclosure. For instance, a count data structure (e.g., a count matrix 110) is obtained for a first plurality of cells (e.g., N cells), collectively representing a plurality of annotated cell states 116. For each respective cell, for each respective cellular constituent in a plurality of cellular constituents 112 (e.g., Z cellular constituents), the count data structure includes a corresponding count 114 of the respective cellular constituent in the respective cell.

Cell States and Processes.

In some embodiments, the cellular process of interest is an aberrant cell process. In some embodiments, the cellular process of interest is a cell process associated with a disease. For example, as described above, in some embodiments, the method provides for the targeting and elucidation of cellular processes and programs that are critical to disease.

In some embodiments, the cellular process of interest is indicative of or related to a mechanism underlying any of the characteristics of disease, including but not limited to onset, progression, symptoms, or severity of disease.

In some embodiments, the disease is selected from the group consisting of infectious or parasitic diseases; neoplasms; diseases of the blood or blood-forming organs; diseases of the immune system; endocrine, nutritional or metabolic diseases; mental, behavioral or neurodevelopmental disorders; sleep-wake disorders; diseases of the nervous system; diseases of the visual system; diseases of the ear or mastoid process; diseases of the circulatory system; diseases of the respiratory system; diseases of the digestive system; diseases of the skin; diseases of the musculoskeletal system or connective tissue; diseases of the genitourinary system; conditions related to sexual health; diseases related to pregnancy, childbirth or the puerperium; certain conditions originating in the perinatal period; and developmental anomalies. In some embodiments, the disease is one or more entries of the ICD-11 MMS, or the International Classification of Disease. The ICD provides a method of classifying diseases, injuries, and causes of death. The World Health Organization (WHO) publishes the ICDs to standardize the methods of recording and tracking instances of diagnosed disease.

In some embodiments, the cellular process of interest is a functional pathway. In some embodiments, the cellular process of interest is a signaling pathway. In some embodiments, the cellular process of interest is a mechanism of action (e.g., of a compound, a small molecule, and/or a therapeutic). In some embodiments the cellular process of interest is a transcriptional network (e.g., a gene regulatory network). Any cellular or biological process known in the art is contemplated for the present disclosure, as well as any aberrations thereof, as will be apparent to one skilled in the art.

In some embodiments, the cellular process of interest is an aberrant cell process associated with a disease, and the first plurality of cells includes cells that are representative of the disease and cells that are not representative of the disease as documented by the plurality of annotated cell states.

In some embodiments, the cellular process of interest is an aberrant cell process associated with a disease, and the first plurality of cells includes cells that are representative of a disease state and cells that are representative of a healthy or control state as documented by the plurality of annotated cell states.

In some embodiments, the cellular process of interest is an aberrant cell process associated with a plurality of diseases, and the first plurality of cells includes a plurality of subsets of cells, each respective subset of cells representative of a respective disease in the plurality of diseases as documented by the plurality of annotated cell states.

In some embodiments, the cellular process of interest is identified by a discrepancy between a diseased state (e.g., a cell obtained from a diseased subject and/or a diseased tissue) and a normal state (e.g., a cell obtained from a healthy or control subject and/or tissue). For instance, in some embodiments, a diseased state is identified by loss of a function of a cell, gain of a function of a cell, progression of a cell (e.g., transition of the cell into a differentiated state), stasis of a cell (e.g., inability of the cell to transition into a differentiated state), intrusion of a cell (e.g., emergence of the cell in an abnormal location), disappearance of a cell (e.g., absence of the cell in a location where the cell is normally present), disorder of a cell (e.g., a structural, morphological, and/or spatial change within and/or around the cell), loss of network of a cell (e.g., a change in the cell that eliminates normal effects in progeny cells or cells downstream of the cell), a gain of network of a cell (e.g., a change in the cell that triggers new downstream effects in progeny cells of cells downstream of the cell), a surplus of a cell (e.g., an overabundance of the cell), a deficit of a cell (e.g., a density of the cell being below a critical threshold), a difference in cellular constituent ratio and/or quantity in a cell, a difference in the rate of transitions in a cell, or any combination thereof.

In some embodiments, the first plurality of cells includes a plurality of subsets of cells, each respective subset of cells representative of a respective disease, functional pathway, signaling pathway, mechanism of action, transcriptional network, discrepancy, and/or cellular or biological process as documented by the plurality of annotated cell states.

In some embodiments, the first plurality of cells includes single cells, cell lines, biopsy sample cells, and/or cultured primary cells. In some embodiments, the first plurality of cells comprises human cells. In some embodiments, the first plurality of cells is obtained from one or more samples as described herein (see, for example, Definitions: Samples).

In some embodiments, the first plurality of cells comprises at least 5, at least 10, at least 15, at least 20, at least 30, at least 40, at least 50, at least 100, at least 200, at least 300, at least 400, at least 500, at least 1000, at least at least 2000, at least 3000, at least 4000, at least 5000, at least 10,000, at least 20,000, at least 30,000, at least 50,000, at least 80,000, at least 100,000, at least 500,000, or at least 1 million cells. In some embodiments, the first plurality of cells comprises no more than 5 million, no more than 1 million, no more than 500,000, no more than 100,000, no more than 50,000, no more than 10,000, no more than 5000, no more than 1000, no more than 500, no more than 200, no more than 100, or no more than 50 cells. In some embodiments, the first plurality of cells comprises from 5 to 100, from 10 to 50, from 20 to 500, from 200 to 10,000, from 1000 to 100,000, from 50,000 to 500,000, or from 10,000 to 1 million cells. In some embodiments, the first plurality of cells falls within another range starting no lower than 5 cells and ending no higher than 5 million cells.

In some embodiments, the plurality of annotated cell states (e.g., cell type/exposure conditions 116 in count data structure 110) comprises one or more of a cell phenotype, cellular behavior, disease state, genetic mutation, perturbations of genes or gene products (e.g., knockdowns, silencing, overexpression, etc.), and/or exposure to a compound.

In some embodiments, an annotated cell state in the plurality of annotated cell states is an exposure of a cell in the first plurality of cells to a compound under an exposure condition. For example, an exposure of a cell includes any treatment of the cell with one or more compounds. In some embodiments, the one or more compounds includes, for example, a small molecule, a biologic, a therapeutic, a protein, a protein combined with a small molecule, an ADC, a nucleic acid (e.g., an siRNA, interfering RNA, cDNA over-expressing wild-type and/or mutant shRNA, cDNA over-expressing wild-type and/or mutant guide RNA (e.g., Cas9 system or other cellular-component editing system, etc.), and/or any combination of any of the foregoing. In some embodiments, the exposure condition is a duration of exposure, a concentration of the compound, or a combination of a duration of exposure and a concentration of the compound.

In some embodiments a compound of the present disclosure is a chemical compound that satisfies the Lipinski rule of five criterion. In some embodiments, a compound of the present disclosure is an organic compounds that satisfies two or more rules, three or more rules, or all four rules of the Lipinski's Rule of Five: (i) not more than five hydrogen bond donors (e.g., OH and NH groups), (ii) not more than ten hydrogen bond acceptors (e.g. N and O), (iii) a molecular weight under 500 Daltons, and (iv) a Log P under 5. The “Rule of Five” is so called because three of the four criteria involve the number five. See, Lipinski, 1997, Adv. Drug Del. Rev. 23, 3, which is hereby incorporated herein by reference in its entirety. In some embodiments, a compound of the present disclosure satisfies one or more criteria in addition to Lipinski's Rule of Five. For example, in some embodiments, a compound of the present disclosure has five or fewer aromatic rings, four or fewer aromatic rings, three or fewer aromatic rings, or two or fewer aromatic rings.

In some embodiments, the plurality of annotated cell states comprises one or more indications of cell batch, cell donor, cell type, cell line, disease status, time points, replicates, and/or relevant metadata. In some embodiments, the plurality of annotated cell states comprises experimental data (e.g., flow cytometry readouts, imaging and microscopy annotations, cellular constituent data, etc.). In some embodiments, the plurality of annotated cell states comprises one or more genetic markers (e.g., copy number variations, single nucleotide variants, multiple nucleotide polymorphisms, insertions, deletions, gene fusions, microsatellite instability status, amplifications, and/or isoforms).

In some embodiments, the plurality of annotated cell states comprises at least 3, at least 5, at least 10, at least 15, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, or at least 3000 cell states. In some embodiments, the plurality of annotated cell states comprises no more than 5000, no more than 1000, no more than 500, no more than 200, no more than 100, no more than 50, or no more than 20 cell states. In some embodiments, the plurality of annotated cell states comprises from 3 to 10, from 10 to 50, from 20 to 500, from 200 to 1000, or from 1000 to 5000 cell states. In some embodiments, the plurality of annotated cell states falls within another range starting no lower than 3 cell states and ending no higher than 5000 cell states.

In some embodiments, the plurality of annotated cell states includes any of the cellular conditions of interest disclosed herein.

In some embodiments, the plurality of annotated cell states includes any of the covariates disclosed herein (see, for example, the section entitled, “Covariates,” below).

Cellular Constituents.

As described above, in some implementations, the method comprises using one or more types of molecular data (e.g., cellular constituents) to elucidate the mechanisms and/or molecular targets underlying cellular processes of interest (e.g., disease-critical cellular processes).

In some embodiments, a cellular constituent is a gene, a gene product (e.g., an mRNA and/or a protein), a carbohydrate, a lipid, an epigenetic feature, a metabolite, and/or a combination thereof. In some embodiments, each cellular constituent in the plurality of cellular constituents is a particular gene, a particular mRNA associated with a gene, a carbohydrate, a lipid, an epigenetic feature, a metabolite, a protein, or a combination thereof.

In some embodiments, the plurality of cellular constituents includes nucleic acids, including DNA, modified (e.g., methylated) DNA, RNA, including coding (e.g., mRNAs) or non-coding RNA (e.g., sncRNAs), proteins, including post-transcriptionally modified protein (e.g., phosphorylated, glycosylated, myristilated, etc. proteins), lipids, carbohydrates, nucleotides (e.g., adenosine triphosphate (ATP), adenosine diphosphate (ADP) and adenosine monophosphate (AMP)) including cyclic nucleotides such as cyclic adenosine monophosphate (cAMP) and cyclic guanosine monophosphate (cGMP), other small molecule cellular constituents such as oxidized and reduced forms of nicotinamide adenine dinucleotide (NADP/NADPH), and any combinations thereof.

In some embodiments, the corresponding abundance of a respective cellular constituent comprises an abundance of any of the cellular constituents disclosed above. In some embodiments, the corresponding abundance of a respective cellular constituent includes gene expression measurements, such as RNA levels.

In some embodiments, the plurality of cellular constituents comprises at least 5, at least 10, at least 15, at least 20, at least 25, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, at least 10,000, at least 20,000, at least 30,000, at least 50,000, or more than 50,000 cellular constituents.

In some embodiments, the plurality of cellular constituents comprises no more than 70,000, no more than 50,000, no more than 30,000, no more than 10,000, no more than 5000, no more than 1000, no more than 500, no more than 200, no more than 100, no more than 90, no more than 80, no more than 70, no more than 60, no more than 50, or no more than 40 cellular constituents.

In some embodiments, the plurality of cellular constituents consists of between twenty and 10,000 cellular constituents. In some embodiments, the plurality of cellular constituents consists of between 100 and 8,000 cellular constituents. In some embodiments, the plurality of cellular constituents comprises from 5 to 20, from 20 to 50, from 50 to 100, from 100 to 200, from 200 to 500, from 500 to 1000, from 1000 to 5000, from 5000 to 10,000, or from 10,000 to 50,000 cellular constituents. In some embodiments, the plurality of cellular constituents falls within another range starting no lower than 5 cellular constituents and ending no higher than 70,000 cellular constituents.

As an example, in some embodiments, the plurality of cellular constituents comprises a plurality of genes, optionally measured at the RNA level. In some embodiments, the plurality of genes comprises at least 5, at least 10, at least 15, at least 20, at least 25, at least 30, at least 40, at least 50, at least 60, at least 70, at least 80, at least 90, at least 100, at least 200, at least 300, at least 400, at least 500, at least 600, at least 700, at least 800, at least 900, or at least 1000 genes. In some embodiments, the plurality of genes comprises at least 1000, at least 2000, at least 3000, at least 4000, at least 5000, at least 10,000, at least 30,000, at least 50,000, or more than 50,000 genes. In some embodiments, the plurality of genes comprises from 5 to 20, from 20 to 50, from 50 to 100, from 100 to 200, from 200 to 500, from 500 to 1000, from 1000 to 5000, from 5000 to 10,000, or from 10,000 to 50,000 genes.

As another example, in some embodiments, the plurality of cellular constituents comprises a plurality of proteins. In some embodiments, the plurality of proteins comprises at least 5, at least 10, at least 15, at least 20, at least 25, at least 30, at least 40, at least 50, at least 60, at least 70, at least 80, at least 90, at least 100, at least 200, at least 300, at least 400, at least 500, at least 600, at least 700, at least 800, at least 900, or at least 1000 proteins. In some embodiments, the plurality of proteins comprises at least 1000, at least 2000, at least 3000, at least 4000, at least 5000, at least 10,000, at least 30,000, at least 50,000, or more than 50,000 proteins. In some embodiments, the plurality of proteins comprises from 5 to 20, from 20 to 50, from 50 to 100, from 100 to 200, from 200 to 500, from 500 to 1000, from 1000 to 5000, from 5000 to 10,000, or from 10,000 to 50,000 proteins.

Any one of a number of abundance counting techniques (e.g., cellular constituent measurement techniques) may be used to obtain the corresponding abundance for each respective cellular constituent in each respective cell (e.g., count 114 in count data structure 110). For instance, Table 1 lists non-limiting techniques for single-cell cellular constituent measurement, in accordance with some embodiments of the present disclosure.

In some embodiments, for instance, gene expression in a respective cell in the first plurality of cells can be measured by sequencing the cell and then counting the quantity of each gene transcript identified during the sequencing. In some embodiments, the gene transcripts sequenced and quantified include RNA, such as mRNA. In some embodiments, the gene transcripts sequenced and quantified include a downstream product of mRNA, such as a protein (e.g., a transcription factor). In general, as used herein, the term “gene transcript” may be used to denote any downstream product of gene transcription or translation, including post-translational modification, and “gene expression” may be used to refer generally to any measure of gene transcripts.

Thus, in some embodiments, the corresponding abundance of the respective cellular constituent is determined using one or more methods including microarray analysis via fluorescence, chemiluminescence, electric signal detection, polymerase chain reaction (PCR), reverse transcriptase polymerase chain reaction (RT-PCR), digital droplet PCR (ddPCR), solid-state nanopore detection, RNA switch activation, a Northern blot, and/or a serial analysis of gene expression (SAGE). In some embodiments, the corresponding abundance of the respective cellular constituent in the respective cell in the first plurality of cells is determined by a colorimetric measurement, a fluorescence measurement, a luminescence measurement, or a resonance energy transfer (FRET) measurement.

For example, in some embodiments, the corresponding abundance of the respective cellular constituent is RNA abundance (e.g., gene expression), and the abundance of the respective cellular constituent is determined by measuring polynucleotide levels of one or more nucleic acid molecules corresponding to the respective gene. The transcript levels of the respective gene can be determined from the amount of mRNA, or polynucleotides derived therefrom, present in the respective cell in the first plurality of cells. Polynucleotides can be detected and quantitated by a variety of methods including, but not limited to, microarray analysis, polymerase chain reaction (PCR), reverse transcriptase polymerase chain reaction (RT-PCR), Northern blot, serial analysis of gene expression (SAGE), RNA switches, RNA fingerprinting, ligase chain reaction, Qbeta replicase, isothermal amplification method, strand displacement amplification, transcription based amplification systems, nuclease protection assays (Si nuclease or RNAse protection assays), and/or solid-state nanopore detection. See, e.g., Draghici, Data Analysis Tools for DNA Microarrays, Chapman and Hall/CRC, 2003; Simon et al., Design and Analysis of DNA Microarray Investigations, Springer, 2004; Real-Time PCR: Current Technology and Applications, Logan, Edwards, and Saunders eds., Caister Academic Press, 2009; Bustin A-Z of Quantitative PCR (IUL Biotechnology, No. 5), International University Line, 2004; Velculescu et al., (1995) Science 270: 484-487; Matsumura et al., (2005) Cell. Microbiol. 7: 11-18; Serial Analysis of Gene Expression (SAGE): Methods and Protocols (Methods in Molecular Biology), Humana Press, 2008; each of which is hereby incorporated herein by reference in its entirety.

In some embodiments, the corresponding abundance of the respective cellular constituent is obtained from expressed RNA or a nucleic acid derived therefrom (e.g., cDNA or amplified RNA derived from cDNA that incorporates an RNA polymerase promoter) from a respective cell in the first plurality of cells, including naturally occurring nucleic acid molecules, as well as synthetic nucleic acid molecules. Thus, in some embodiments, the corresponding abundance of the respective cellular constituent is obtained from such non-limiting sources as total cellular RNA, poly(A)+ messenger RNA (mRNA) or a fraction thereof, cytoplasmic mRNA, or RNA transcribed from cDNA (e.g., cRNA). Methods for preparing total and poly(A)+RNA are well known in the art, and are described generally, e.g., in Sambrook, et al., Molecular Cloning: A Laboratory Manual (3rd Edition, 2001). RNA can be extracted from a cell of interest using guanidinium thiocyanate lysis followed by CsCl centrifugation (see, e.g., Chirgwin et al., 1979, Biochemistry 18:5294-5299), a silica gel-based column (e.g., RNeasy (Qiagen, Valencia, Calif.) or StrataPrep (Stratagene, La Jolla, Calif.)), or using phenol and chloroform, as described in Ausubel et al., eds., 1989, Current Protocols In Molecular Biology, Vol. III, Green Publishing Associates, Inc., John Wiley & Sons, Inc., New York, at pp. 13.12.1-13.12.5). Poly(A)+RNA can be selected, e.g., by selection with oligo-dT cellulose or, alternatively, by oligo-dT primed reverse transcription of total cellular RNA. RNA can be fragmented by methods known in the art, e.g., by incubation with ZnC12, to generate fragments of RNA.

In some embodiments, the corresponding abundance of the respective cellular constituent in the respective cell in the first or second plurality of cells is determined by sequencing.

In some embodiments, the corresponding abundance of the respective cellular constituent in the respective cell in the first or second plurality of cells is determined by single-cell ribonucleic acid (RNA) sequencing (scRNA-seq), scTag-seq, single-cell assay for transposase-accessible chromatin using sequencing (scATAC-seq), CyTOF/SCoP, E-MS/Abseq, miRNA-seq, CITE-seq, and any combination thereof. The cellular constituent abundance measurement technique can be selected based on the desired cellular constituent to be measured. For instance, scRNA-seq, scTag-seq, and miRNA-seq can be used to measure RNA expression. Specifically, scRNA-seq measures expression of RNA transcripts, scTag-seq allows detection of rare mRNA species, and miRNA-seq measures expression of micro-RNAs. CyTOF/SCoP and E-MS/Abseq can be used to measure protein expression in the cell. CITE-seq simultaneously measures both gene expression and protein expression in the cell, and scATAC-seq measures chromatin conformation in the cell. Table 1 below provides example protocols for performing each of the cellular constituent abundance measurement techniques described above.

TABLE 1
Example Measurement Protocols

| Technique | Protocol |
|---|---|
| RNA-seq | Olsen et al., (2018), “Introduction to Single-Cell RNA |
| | Sequencing,” Current protocols in molecular biology |
| | 122(1), pg. 57. |
| Tag-seq | Rozenberg et al., (2016), “Digital gene expression |
| | analysis with sample multiplexing and PCR duplicate |
| | detection: A straightforward protocol,” BioTechniques, |
| | 61(1), pg. 26. |
| ATAC-seq | Buenrostro et al., (2015), “ATAC-seq: a method for |
| | assaying chromatic accessibility genome-wide,” Current |
| | protcols in molecular biology, 109(1), pg. 21. |
| miRNA-seq | Faridani et al., (2016), “Single-cell sequencing of the |
| | small-RNA transcriptome,” Nature biotechnology, |
| | 34(12), pg. 1264. |
| CyTOF/SCoPE-MS/Abseq | Bandura et al., (2009), “Mass cytometry: technique for |
| | real time single cell multitarget immunoassay based on |
| | inductivitely coupled plasma time-of-flight mass |
| | spectrometry,” Analystic chemistry, 81(16), pg. 6813. |
| | Budnik et al., (2018), “SCoPE-ME: mass scpectrometry |
| | of single mammalian cells quantifies proteome |
| | heterogenity during cell differentiation,” Genome |
| | biology, 19(1), pg. 161. |
| | Shahi et al., (2017), “Abseq: Ultrahigh-throughoutput |
| | single cell protein profiling with droplep microfluidic |
| | barcoding,” Scientific reports, 7, pg. 44447. |
| CITE-seq | Stoeckius et al., (2017), “Simultaneous epitope and |
| | transcritome measurement in single cells,” Nature |
| | Methods, 14(9), pg. 856. |

In some embodiments, the plurality of cellular constituents is measured at a single time point. In some embodiments, the plurality of cellular constituents is measured at multiple time points. For instance, in some embodiments, the plurality of cellular constituents is measured at multiple time points throughout a cell state transition (e.g., a differentiation process, a response to an exposure to a compound, a developmental process, etc.).

It is to be understood that this is by way of illustration and not limitation, as the present disclosure encompasses analogous methods using measurements of other cellular constituents obtained from cells (e.g., single cells). It is to be further understood that the present disclosure encompasses methods using measurements obtained directly from experimental work carried out by an individual or organization practicing the methods described in this disclosure, as well as methods using measurements obtained indirectly, e.g., from reports of results of experimental work carried out by others and made available through any means or mechanism, including data reported in third-party publications, databases, assays carried out by contractors, or other sources of suitable input data useful for practicing the disclosed methods.

All of the techniques described herein are equally applicable to any technique that obtains data from one or more cells (e.g., single cells) in the plurality of cells for the one or more first datasets. Examples include gene transcripts and gene expression, proteomics (protein expression), chromatin conformation (chromatin status), methylation, or other quantifiable epigenetic effects.

In some embodiments, the one or more first datasets comprises the same plurality of cellular constituents for each respective cell in the first plurality of cells. In some embodiments, the one or more first datasets comprises, for two or more respective cells in the first plurality of cells, a different plurality of cellular constituents. In some embodiments, the corresponding abundance of each respective cellular constituent for a respective cell in the first plurality of cells is measured only from the respective cell (e.g., using a single-cell measurement technique). In some embodiments, the corresponding abundance of each respective cellular constituent for a respective cell in the first plurality of cells is measured from a plurality of cells.

In some embodiments, the obtaining the one or more first datasets comprises preprocessing the corresponding abundances for the plurality of cellular constituents. In some embodiments, the preprocessing includes one or more of filtering, normalization, mapping (e.g., to a reference sequence), quantification, scaling, deconvolution, cleaning, dimension reduction, transformation, statistical analysis, and/or aggregation.

For example, in some embodiments, the plurality of cellular constituents is filtered based on a desired quality, e.g., size and/or quality of a nucleic acid sequence, or a minimum and/or maximum abundance value for a respective cellular constituent. In some embodiments, filtering is performed in part or in its entirety by various software tools, such as Skewer. See, Jiang, H. et al., BMC Bioinformatics 15(182):1-12 (2014). In some embodiments, the plurality of cellular constituents is filtered for quality control, for example, using a sequencing data QC software such as AfterQC, Kraken, RNA-SeQC, FastQC, or another similar software program. In some embodiments, the plurality of cellular constituents is normalized, e.g., to account for pull-down, amplification, and/or sequencing bias (e.g., mappability, GC bias etc.). See, for example, Schwartz et al., PLoS ONE 6(1):e16685 (2011) and Benjamini and Speed, Nucleic Acids Research 40(10):e72 (2012), the contents of which are hereby incorporated by reference, in their entireties, for all purposes. In some embodiments, the preprocessing removes a subset of cellular constituents from the plurality of cellular constituents.

In some embodiments, the obtaining the one or more first datasets comprises preprocessing the corresponding abundances for the plurality of cellular constituents to improve (e.g., lower) a high signal-to-noise ratio.

In some embodiments, the preprocessing comprises performing a comparison of a corresponding abundance of a respective cellular constituent in a respective cell to a reference abundance. In some embodiments, the reference abundance is obtained from, e.g., a normal sample, a matched sample, a reference dataset comprising reference abundance values, a reference cellular constituent such as a housekeeping gene, and/or a reference standard. In some embodiments, this comparison of cellular constituent abundances is performed using any differential expression test including, but not limited to, a difference of means test, a Wilcoxon rank-sum test (Mann Whitney U test), a t-test, a logistic regression, and a generalized linear model. Those of skill in the art will appreciate that other metrics are also possible for comparison and/or normalization of cellular constituent abundances.

Thus, in some embodiments, the corresponding abundance of a respective cellular constituent in a respective cell in the first dataset comprises any one of a variety of forms, including, without limitation, a raw abundance value, an absolute abundance value (e.g., transcript number), a relative abundance value (e.g., relative fluorescent units, transcriptome analysis, and/or gene set expression analysis (GSEA)), a compound or aggregated abundance value, a transformed abundance value (e.g., log 2 and/or log 10 transformed), a change (e.g., fold- or log-change) relative to a reference (e.g., a normal sample, matched sample, reference dataset, housekeeping gene, and/or reference standard), a standardized abundance value, a measure of central tendency (e.g., mean, median, mode, weighted mean, weighted median, and/or weighted mode), a measure of dispersion (e.g., variance, standard deviation, and/or standard error), an adjusted abundance value (e.g., normalized, scaled, and/or error-corrected), a dimension-reduced abundance value (e.g., principal component vectors and/or latent components), and/or a combination thereof. Methods for obtaining cellular constituent abundances using dimension reduction techniques are known in the art and further detailed below, including but not limited to principal component analysis, factor analysis, linear discriminant analysis, multi-dimensional scaling, isometric feature mapping, locally linear embedding, hessian eigenmapping, spectral embedding, t-distributed stochastic neighbor embedding, and/or any substitutions, additions, deletions, modification, and/or combinations thereof as will be apparent to one skilled in the art. See, for example, Sumithra et al., 2015, “A Review of Various Linear and Non Linear Dimensionality Reduction Techniques,” Int J Comp Sci and Inf Tech, 6(3), 2354-2360, which is hereby incorporated herein by reference in its entirety.

Referring to Block 204, the method further includes accessing or forming a plurality of vectors, each respective vector in the plurality of vectors (i) corresponding to a respective cellular constituent in the plurality of constituents and (ii) comprising a corresponding plurality of elements, each respective element in the corresponding plurality of elements having a corresponding count representing the corresponding abundance of the respective cellular constituent in a respective cell in the first plurality of cells.

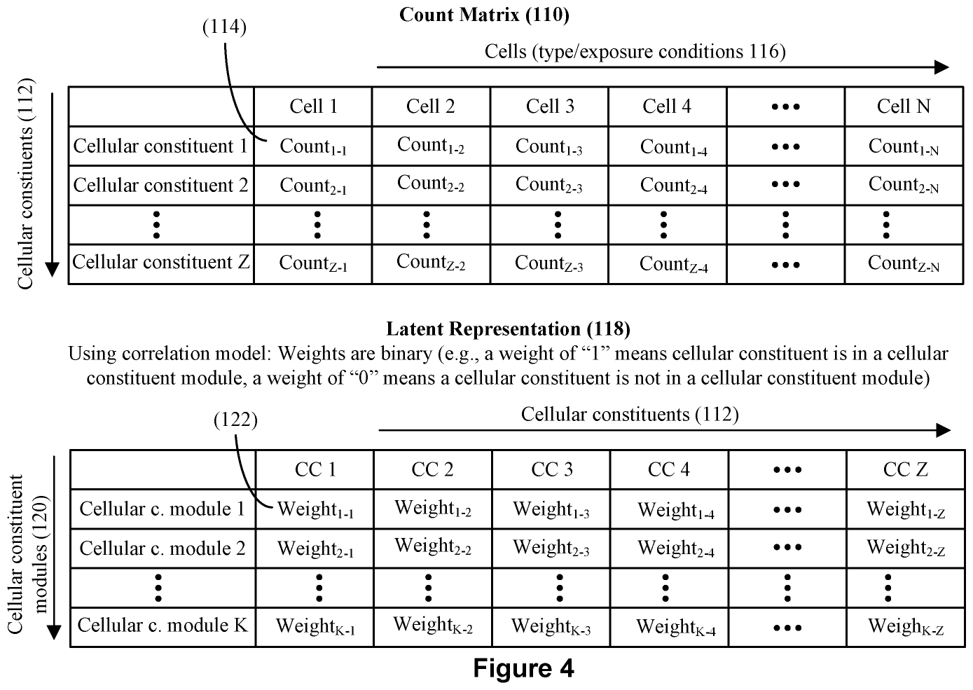

For instance, referring again to FIG. 1B, the count data structure 110 includes a first plurality of N cells and a plurality of Z cellular constituents. The count data structure 110 can be represented as a count matrix, which is further illustrated in FIG. 4 (top panel). Each cell in the first plurality of N cells is arranged along the x-axis (columns) and each cellular constituent in the plurality of Z cellular constituents are arranged along the y-axis (rows). Each row forms a respective vector for the corresponding cellular constituent, where each entry for each respective column in the row is an abundance (e.g., a count) of the cellular constituent for the respective cell corresponding to the respective column. Thus, as illustrated in FIG. 4, the vector corresponding to cellular constituent 1 includes count₁₋₁ corresponding to cell 1, count₁₋₂ corresponding to cell 2, and so on.

In some embodiments, each respective cellular constituent in the plurality of cellular constituents is a gene, and each gene is represented by an expression vector in which each entry in the vector represents the abundance of the gene in each cell or each set of cells having one or more conditions of interest (e.g., annotated cell states).

Cellular Constituent Modules.

When associated with a cellular process, modules of cellular constituents (e.g., genes) can be thought to arise from a sequence of switching events, where cellular constituents (e.g., genes) that switch at similar times form a module together. Thus, for instance, in some embodiments, each respective vector in the plurality of vectors is an expression vector for a gene, in which each entry in the vector represents expression of the gene in a respective cell (or set of cells) associated with a condition of interest (e.g., a disease condition versus a healthy condition). Cellular constituent modules therefore can be found based on similarities in gene expression patterns between different conditions (e.g., groups of genes with similar expression patterns across a plurality of different cells).

In some embodiments, a cellular constituent is a gene, a gene product (e.g., an mRNA and/or a protein), a carbohydrate, a lipid, an epigenetic feature, a metabolite, and/or a combination thereof. In some embodiments, each cellular constituent in the plurality of cellular constituents is a particular gene, a particular mRNA associated with a gene, a carbohydrate, a lipid, an epigenetic feature, a metabolite, a protein, or a combination thereof. In some embodiments, the plurality of cellular constituents includes nucleic acids, including DNA, modified (e.g., methylated) DNA, RNA, including coding (e.g., mRNAs) or non-coding RNA (e.g., sncRNAs), proteins, including post-transcriptionally modified protein (e.g., phosphorylated, glycosylated, myristilated, etc. proteins), lipids, carbohydrates, nucleotides (e.g., adenosine triphosphate (ATP), adenosine diphosphate (ADP) and adenosine monophosphate (AMP)) including cyclic nucleotides such as cyclic adenosine monophosphate (cAMP) and cyclic guanosine monophosphate (cGMP), other small molecule cellular constituents such as oxidized and reduced forms of nicotinamide adenine dinucleotide (NADP/NADPH), and any combinations thereof.

Accordingly, referring to Block 206, the method includes using the plurality of vectors to identify each cellular constituent module in a plurality of cellular constituent modules (e.g., comprising more than ten cellular constituent modules). Each cellular constituent module in the plurality of cellular constituent modules includes a subset of the plurality of cellular constituents, where the plurality of cellular constituent modules are arranged in a latent representation dimensioned by (i) the plurality of cellular constituent modules and (ii) the plurality of cellular constituents or a representation thereof.

In some embodiments, the plurality of cellular constituent modules comprises at least 5, at least 10, at least 15, at least 20, at least 25, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, at least 3000, at least 4000, or at least 5000 cellular constituent modules. In some embodiments, the plurality of cellular constituent modules comprises no more than 10,000, no more than 5000, no more than 2000, no more than 1000, no more than 500, no more than 300, no more than 200, no more than 100, no more than 90, no more than 80, no more than 70, no more than 60, or no more than 50 cellular constituent modules.

In some embodiments, the plurality of cellular constituent modules consists of between 10 and 2000 cellular constituent modules. In some embodiments, the plurality of cellular constituent modules consists of between 50 and 500 cellular constituent modules. In some embodiments, the plurality of cellular constituent modules comprises from 5 to 20, from 20 to 50, from 50 to 100, from 100 to 200, from 200 to 500, from 500 to 1000, from 1000 to 5000, or from 5000 to 10,000 cellular constituent modules. In some embodiments, the plurality of cellular constituent modules falls within another range starting no lower than 5 cellular constituent modules and ending no higher than 10,000 cellular constituent modules.

In some embodiments, each cellular constituent module in the plurality of cellular constituent modules comprises the same or a different number of cellular constituents in the respective subset of the plurality of cellular constituents. In some embodiments, each respective subset of cellular constituents corresponding to each respective cellular constituent module has a unique subset of cellular constituents (e.g., each cellular constituent is grouped into no more than one module). In some embodiments, a first cellular constituent module has a first subset of cellular constituents that overlaps a second subset of cellular constituents corresponding to a second cellular constituent module (e.g., a cellular constituent is common to two or more different modules).

In some embodiments, a respective cellular constituent module in the plurality of cellular constituent modules comprises at least 2, at least 5, at least 10, at least 15, at least 20, at least 25, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, or at least 3000 cellular constituents. In some embodiments, a respective cellular constituent module in the plurality of cellular constituent modules comprises no more than 5000, no more than 3000, no more than 1000, no more than 500, no more than 200, no more than 100, no more than 90, no more than 80, no more than 70, no more than 60, or no more than 50 cellular constituents.

In some embodiments, a respective cellular constituent module in the plurality of cellular constituent modules comprises from 5 to 100, from 2 to 300, from 20 to 500, from 200 to 1000, or from 1000 to 5000 cellular constituents. In some embodiments, the plurality of cellular constituents in a respective cellular constituent module falls within another range starting no lower than 2 cellular constituents and ending no higher than 5000 cellular constituents.

In some embodiments, each cellular constituent module in the plurality of constituent modules consists of between two and three hundred cellular constituents.

In some embodiments, the using the plurality of vectors to identify each cellular constituent module in a plurality of cellular constituent modules comprises application of a correlation model to the plurality of vectors using each corresponding plurality of elements of each vector in the plurality of vectors. In some embodiments, the correlation model includes a clustering method (e.g., a clustering algorithm). In some embodiments, the correlation model includes a graph clustering method (e.g., algorithm) and/or a non-graph clustering method. In some embodiments, the graph clustering method is Leiden clustering on a Pearson-correlation-based distance metric. In some embodiments, the graph clustering method is Louvain clustering.

For example, in some implementations, the method comprises application of a correlation-based cost function. Optimizing a correlation-based cost function includes computing a nearest-neighbor graph defining neighborhood relations among cellular constituents (e.g., genes), representing each cellular constituent by a vector formed by storing the abundance counts (e.g., expression values) for the cellular constituent in each cell, and computing correlations among cellular constituents. Cellular constituents with high correlations among one another are determined to be nearest neighbors, and are used to form a cellular constituent module by clustering the graph using a graph clustering method (e.g., Leiden and/or Louvain).

Any one of a number of clustering techniques can be used, examples of which include, but are not limited to, hierarchical clustering, k-means clustering, and density based clustering. In an embodiment, a hierarchical density based clustering algorithm is used (referred to as HDBSCAN, see, e.g., Campello et al., (2015). Hierarchical density estimates for data clustering, visualization, and outlier detection. ACM Trans Knowl Disc Data, 10(1), 5). In another embodiment, a community detection based cluster algorithm is used, such as Louvain clustering (see, e.g., Blondel et al., (2008). Fast unfolding of communities in large networks. J stat mech: theor exp, 2008(10), P10008). In yet another embodiment, Leiden clustering is used. The Leiden algorithm proceeds by moving individual nodes between communities to determine partitions, refining partitions, and creating aggregate networks based on the refined partitions. Aggregate networks are further partitioned based on the unrefined partitions determined in earlier steps of the process, and new partitions are refined by moving individual nodes within each aggregate network. See, e.g., Traag et al., (2019), “From Louvain to Leiden: guaranteeing well-connected communities,” Sci Rep 9:5233, doi: 10.1038/s41598-019-41695-z. In still another embodiment, a diffusion path algorithm is used.

Generally, clustering algorithms such as Louvain clustering and/or Leiden clustering use hard partitioning techniques, in which each element (e.g., each cellular constituent) is uniquely assigned to a single cluster without overlapping. However, and without being bound to any one particular theory, cellular processes may be characterized by complex and dynamic interactions between networks of cellular constituents within the cell, where a single gene, for instance, can play a role in two, three, four, or more cellular processes within a cell, in combination with any number of other genes that similarly function in any number of the same or different processes and pathways. Thus, in paralleling the complexity of intracellular activity, the clustering of cellular constituents into a first module need not necessarily be exclusive of any other module. In some embodiments, therefore, the identification of cellular constituent modules comprises obtaining modules with overlapping subsets of cellular constituents.

Instead of using a hard partitioning technique via a correlation based model, in some embodiments, the using the plurality of vectors to identify each cellular constituent module in a plurality of cellular constituent modules comprises a dictionary learning model that produces the representation of the plurality of cellular constituents as a plurality of dimension reduction components. In some embodiments, the dictionary learning model is L0-regularized autoencoder. An advantage of these models is that they do not enforce a 1:1 correspondence between modules and cellular constituents, but allow cellular constituents to appear in several modules at the same time.

For example, in some implementations, the method comprises application of a spare autoencoder cost function. In some such instances, optimizing a sparse autoencoder cost function includes training a one-layer autoencoder with L0 regularization of its weights, and a reconstruction loss, using standard training algorithms as implemented in pytorch or tensorflow.

Other methods of overlapping partitioning algorithms are possible, including, but not limited to, fuzzy K-means, overlapping K-means (OKM), weighted OKM (WOKM), overlapping partitioning cluster (OPC), and multi-cluster overlapping K-means extension (MCOKE), and/or any variations or combinations thereof

Latent Representations.

In some embodiments, statistical techniques can be used to compress high dimensional data (e.g., abundances of a plurality of cellular constituents across a plurality of cellular constituent modules, for each cell in the first plurality of cells collectively representing a plurality of annotated cell states) to a lower dimensional space, while preserving the shape of the latent information encoded in the one or more first datasets (e.g., a count matrix 110 dimensionality reduced to a latent representation 118, as illustrated in FIGS. 1B, 1C, and 4). The data reduced to the lower dimensional space represents the clustering of cellular constituents across the first plurality of cells, based on similarities of their corresponding abundances under conditions of different annotated cell states (e.g., cell types, exposure conditions, diseases, etc.) and thus can be used to indicate associations between cellular constituents and cell states.

In some embodiments, the latent representation for the plurality of cellular constituent modules is obtained using a dimensionality reduction. For instance, in some implementations, the dimensionality reduction is performed by the computing device (e.g., system 100) to reduce the dimensionality of the data while preserving the structure of any latent patterns that are present in the one or more first datasets.

As illustrated in FIGS. 1B and 4, the input for dimensionality reduction is generally a matrix such as the count matrix 110, in which the count vectors for each cell in the first plurality of cells is concatenated for each cellular constituent (e.g., cellular constituent vector 112 of FIG. 1B). In some implementations, the output of the dimensionality reduction is also a matrix, as illustrated by the latent representation 118 of FIGS. 1C and 4. The latent representation encodes the original data from the one or more first datasets into a compressed form while maintaining the underlying latent structure of the data. Referring to FIG. 4, each row in the matrix is associated with a respective cellular constituent module in the plurality of K cellular constituent modules. Each column in the matrix is associated with one of the dimensions in the reduced dimensional space provided by the dimensionality reduction. For instance, in some embodiments, each dimension corresponds to a respective cellular constituent (e.g., in a plurality of Z cellular constituents) or a representation thereof.

Referring again to the latent representation illustrated in FIG. 4, the values in the entries at each row-column grouping are determined by the dimensionality reduction based on the original input datasets. For instance, each entry can include an indication of membership, for each respective cellular constituent represented by the respective column, in the subset of the plurality of cellular constituents included in the respective cellular constituent module represented by the respective row (e.g., weight₁₋₁, weight₁₋₂, etc.). In particular, in some embodiments, each entry is a weight indicating whether the respective cellular constituent is included in the respective module. In some implementations, a weight is a binary indication of membership (e.g., presence or absence in a respective module is indicated by a 1 or a 0, respectively). In some implementations, a weight is scaled to indicate a relative importance of a cellular constituent to a respective module (e.g., a probability of membership and/or a correlation).

As described above, in some embodiments, a respective dimension in the latent representation corresponds to a representation of a respective cellular constituent. Representations of cellular constituents can arise, for example, from nonlinear representations of cellular constituents, such as where a respective entry (e.g., weight) in a latent representation matrix corresponds to a plurality of cellular constituents. Other embodiments comprising representations of cellular constituent include latent representations obtained using principal component analysis, in which each principal component represent variance and/or other transformations of the data corresponding to the plurality of cellular constituents.

In some embodiments, dimensionality reduction techniques result in some lossy compression of the data. However, the resulting latent representation (e.g., latent representation 118) is smaller in computational storage size, and therefore requires less computing processing power to analyze in conjunction with other downstream techniques such as model training. The arrangement of the plurality of cellular constituent modules in a latent representation thus increases the computational feasibility of the presently disclosed method, using computing devices of the current era.

A variety of dimensionality reduction techniques may be used. Examples include, but are not limited to, principal component analysis (PCA), non-negative matrix factorization (NMF), linear discriminant analysis (LDA), diffusion maps, or network (e.g., neural network) techniques such as an autoencoder.

In some embodiments, the dimension reduction is a principal components algorithm, a random projection algorithm, an independent component analysis algorithm, or a feature selection method, a factor analysis algorithm, Sammon mapping, curvilinear components analysis, a stochastic neighbor embedding (SNE) algorithm, an Isomap algorithm, a maximum variance unfolding algorithm, a locally linear embedding algorithm, a t-SNE algorithm, a non-negative matrix factorization algorithm, a kernel principal component analysis algorithm, a graph-based kernel principal component analysis algorithm, a linear discriminant analysis algorithm, a generalized discriminant analysis algorithm, a uniform manifold approximation and projection (UMAP) algorithm, a LargeVis algorithm, a Laplacian Eigenmap algorithm, or a Fisher's linear discriminant analysis algorithm. See, for example, Fodor, 2002, “A survey of dimension reduction techniques,” Center for Applied Scientific Computing, Lawrence Livermore National, Technical Report UCRL-ID-148494; Cunningham, 2007, “Dimension Reduction,” University College Dublin, Technical Report UCD-CSI-2007-7, Zahorian et al., 2011, “Nonlinear Dimensionality Reduction Methods for Use with Automatic Speech Recognition,” Speech Technologies. doi:10.5772/16863. ISBN 978-953-307-996-7; and Lakshmi et al., 2016, “2016 IEEE 6th International Conference on Advanced Computing (IACC),” pp. 31-34. doi:10.1109/IACC.2016.16, ISBN 978-1-4673-8286-1, each of which is hereby incorporated by reference. Accordingly, in some embodiments, the dimension reduction is a principal component analysis (PCA) algorithm, and each respective extracted dimension reduction component comprises a respective principal component derived by the PCA. In such embodiments, the number of principal components in the plurality of principal components can be limited to a threshold number of principal components calculated by the PCA algorithm. The threshold number of principal components can be, for example, at least 5, at least 10, at least 20, at least 50, at least 100, at least 1000, at least 1500, or any other number. In some embodiments, each principal component calculated by the PCA algorithm is assigned an eigenvalue by the PCA algorithm, and the corresponding subset of the first plurality of extracted features is limited to the threshold number of principal components assigned the highest eigenvalues. For each respective cellular constituent vector in the plurality of cellular constituent vectors, the plurality of dimension reduction components are applied to the respective cellular constituent vector to form a corresponding dimension reduction vector that includes a dimension reduction component value for each respective dimension reduction component in the plurality of dimension reduction components. This forms, from the plurality of cellular constituent vectors, a corresponding plurality of dimension reduction vectors, thereby forming a plurality of cellular constituent modules arranged in a latent representation.

In some embodiments, the method further includes performing manifold learning using the plurality of cellular constituent modules arranged in a latent representation. Generally, manifold learning is used to describe the low-dimensional structure of high-dimensional data by determining maximal variations in a dataset. Examples include, but are not limited to, force-directed layout (Fruchterman and Reingold, 1991, “Graph drawing by force-directed placement,” Software: Practice and experience 21(11), 1129-1164) (e.g., Force Atlas 2), t-distributed stochastic neighbor embedding (t-SNE), locally linear embedding (Roweis, S. T., & Saul, L. K. (2000). Nonlinear dimensionality reduction by locally linear embedding. Science, 290(5500), 2323-2326), local linear isometric mapping (ISOMAP, Tenenbaum, J. B., De Silva, V., & Langford, J. C. (2000). A global geometric framework for nonlinear dimensionality reduction. Science, 290(5500), 2319-2323), kernel PCA, graph-based kernel PCA, Potential of Heat-Diffusion for Affinity Based Trajectory Embedding (PHATE), generalized discriminant analysis (GDA), Uniform Manifold Approximation and Projection (UMAP), or kernel discriminant analysis. Discriminant analysis may be used particularly where some information is known in advance as to the specific cell type of each cell. Force-directed layouts are useful in various particular embodiments because of their ability to identify new, lower dimensions that encode non-linear aspects of the underlying data which arise from underlying cellular processes. Force directed layouts use physics-based models as mechanisms for determining a reduced dimensionality that best represents the data. As an example, a force directed layout uses a form of physics simulation in which, in this embodiment, each cell in the one or more first datasets is assigned a “repulsion” force and there exists a global “gravitation force” that, when computed over the first plurality of cells, identifies sectors of the data that “diffuse” together under these competing “forces.” Force directed layouts make few assumptions about the structure of the data, and do not impose a de-noising approach.

Manifold learning is further described, for example, in Wang et al., 2004, “Adaptive Manifold Learning,” Advances in Neural Information Processing Systems 17, which is hereby incorporated herein by reference in its entirety.

Covariates.

Referring to Block 208, the method further comprises obtaining one or more second datasets in electronic form, the one or more second datasets comprising or collectively comprising, for each respective cell in a second plurality of cells, where the second plurality of cells comprises, e.g., twenty or more cells and collectively represents a plurality of covariates possibly informative of the cellular process of interest, for each respective cellular constituent in the plurality of cellular constituents, a corresponding abundance of the respective cellular constituent in the respective cell. Thus, a cellular constituent count data structure is obtained, where the cellular constituent count data structure is dimensioned by (i) the second plurality of cells and (ii) the plurality of cellular constituents or the representation thereof.

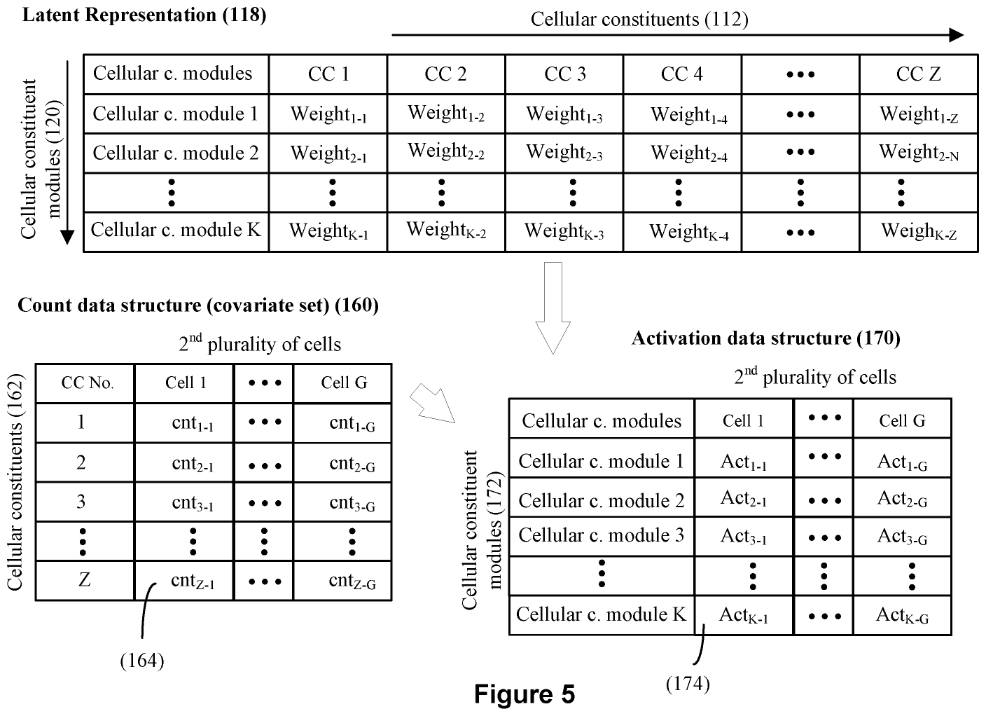

For instance, referring to FIG. 1D, a cellular constituent count data structure 160 collectively representing a plurality of covariates (e.g., count data structure—covariate set 160) includes a first plurality of cells (e.g., G cells), collectively representing a plurality of covariates 166. For each respective cell, for each respective cellular constituent in the plurality of cellular constituents 162 (e.g., Z cellular constituents), the cellular constituent count data structure 160 includes a corresponding count 164 of the respective cellular constituent in the respective cell. The cellular constituent count data structure 160 can additionally be represented as a matrix, which is further illustrated by the lower left panel of FIG. 5. Each cell in the first plurality of G cells is arranged along the x-axis (columns) and each cellular constituent in the plurality of Z cellular constituents are arranged along the y-axis (rows). Each entry for each respective column in each respective row is an abundance (e.g., a count) of the cellular constituent for the respective cell corresponding to the respective column. Thus, as illustrated in FIG. 5, the counts corresponding to cellular constituent 1 includes count₁₋₁ corresponding to cell 1, count_{1-G} corresponding to cell G, and so on.

In some embodiments, the plurality of covariates comprises cell batch, cell donor, cell type, or disease status of one or more cells in the second plurality of cells.

In some embodiments, the plurality of covariates comprises one or more indications of time points, replicates, and/or relevant metadata related to one or more cells in the second plurality of cells. In some embodiments, the plurality of covariates comprises experimental data (e.g., flow cytometry readouts, imaging and microscopy annotations, cellular constituent data, etc.). In some embodiments, the plurality of covariates comprises one or more genetic markers characteristic of one or more cells in the second plurality of cells (e.g., copy number variations, single nucleotide variants, multiple nucleotide polymorphisms, insertions, deletions, gene fusions, microsatellite instability status, amplifications, and/or isoforms).

In some embodiments, the plurality of covariates comprises one or more of a cell phenotype, cellular behavior, disease state, genetic mutation, perturbations of genes or gene products (e.g., knockdowns, silencing, overexpression, etc.), and/or exposure condition for one or more cells in the second plurality of cells.

For example, in some embodiments, a covariate is an exposure or a response to an exposure of a cell in the second plurality of cells to a compound under an exposure condition. In some embodiments, an exposure of a cell includes any treatment of the cell with one or more compounds. In some embodiments, the one or more compounds includes, for example, a small molecule, a biologic, a therapeutic, a protein, a protein combined with a small molecule, an ADC, a nucleic acid (e.g., an siRNA, interfering RNA, cDNA over-expressing wild-type and/or mutant shRNA, cDNA over-expressing wild-type and/or mutant guide RNA (e.g., Cas9 system or other cellular-component editing system), etc.), and/or any combination of any of the foregoing. In some embodiments, the exposure condition is a duration of exposure, a concentration of the compound, or a combination of a duration of exposure and a concentration of the compound. In some embodiments, a covariate is a compound applied to one or more cells that induces a cell state transition and/or a perturbation signature in the one or more cells (e.g., a perturbagen).

In some embodiments, a covariate is a knowledge term (e.g., an annotation) associated with a cellular constituent in the plurality of cellular constituents, or with a cell in the second plurality of cells. For instance, in some embodiments, a covariate is a genome-wide association study (GWAS) annotation, a gene set enrichment assay (GSEA) annotation, a gene ontology annotation, a functional and/or signaling pathway annotation, and/or a cellular signature annotation. In some embodiments, a covariate is obtained from any public knowledge database known in the art, including, but not limited to, NIH Gene Expression Omnibus (GEO), EBI ArrayExpress, NCBI, BLAST, EMBL-EBI, GenBank, Ensembl, the KEGG pathway database, and/or any disease-specific database. In some embodiments, a covariate is obtained from a database providing perturbation (e.g., small-molecule) induced gene expression signatures, such as the Library of Integrated Network-based Cellular Signatures (LINCS) L1000 dataset. See, e.g., Duan, 2016, “L1000CDS²: An ultra-fast LINCS L1000 Characteristic Direction Signature Search Engine,” Systems Biology and Applications 2, article 16015, which is hereby incorporated herein by reference in its entirety.

In some embodiments, the plurality of covariates comprises at least 3, at least 5, at least 10, at least 15, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, or at least 3000 covariates. In some embodiments, the plurality of covariates comprises no more than 5000, no more than 1000, no more than 500, no more than 200, no more than 100, no more than 50, or no more than 20 covariates. In some embodiments, the plurality of covariates comprises from 3 to 10, from 10 to 50, from 20 to 500, from 200 to 1000, or from 1000 to 5000 covariates. In some embodiments, the plurality of covariates falls within another range starting no lower than 3 covariates and ending no higher than 5000 covariates.

In some embodiments, any of the methods and/or embodiments for obtaining the one or more first datasets including the first plurality of cells and the corresponding abundances for the plurality of cellular constituents, described herein in the above sections entitled “Cell states and processes” and “Cellular constituents,” are applicable with respect to the methods and/or embodiments for obtaining the one or more second datasets including the second plurality of cells and the corresponding abundances for the plurality of cellular constituents.

For instance, in some embodiments, the corresponding abundance of the respective cellular constituent in the respective cell in the second plurality of cells is determined by a colorimetric measurement, a fluorescence measurement, a luminescence measurement, or a resonance energy transfer (FRET) measurement. In some embodiments, the corresponding abundance of the respective cellular constituent in the respective cell in the second plurality of cells is determined by single-cell ribonucleic acid (RNA) sequencing (scRNA-seq), scTag-seq, single-cell assay for transposase-accessible chromatin using sequencing (scATAC-seq), CyTOF/SCoP, E-MS/Abseq, miRNA-seq, CITE-seq, and any combination thereof.

In some embodiments, the obtaining the one or more second datasets comprises preprocessing the corresponding abundances for the plurality of cellular constituents for each respective cell in the second plurality of cells, using any of the methods and/or embodiments disclosed herein.

In some embodiments, the one or more first datasets and the one or more second datasets comprise abundances for a plurality of cellular constituents, where the plurality of cellular constituents is the same for the one or more first datasets and the one or more second datasets. In some embodiments, the plurality of cellular constituents for the one or more first datasets is different from the plurality of cellular constituents for the one or more second datasets.

In some embodiments, the second plurality of cells does not include any cells included in the first plurality of cells. In some embodiments, the second plurality of cells includes some or all of the cells included in the first plurality of cells.

In some embodiments, the second plurality of cells comprises at least 5, at least 10, at least 15, at least 20, at least 30, at least 40, at least 50, at least 100, at least 200, at least 300, at least 400, at least 500, at least 1000, at least at least 2000, at least 3000, at least 4000, at least 5000, at least 10,000, at least 20,000, at least 30,000, at least 50,000, at least 80,000, at least 100,000, at least 500,000, or at least 1 million cells. In some embodiments, the second plurality of cells comprises no more than 5 million, no more than 1 million, no more than 500,000, no more than 100,000, no more than 50,000, no more than 10,000, no more than 5000, no more than 1000, no more than 500, no more than 200, no more than 100, or no more than 50 cells. In some embodiments, the second plurality of cells comprises from 5 to 100, from 10 to 50, from 20 to 500, from 200 to 10,000, from 1000 to 100,000, from 50,000 to 500,000, or from 10,000 to 1 million cells. In some embodiments, the second plurality of cells falls within another range starting no lower than 5 cells and ending no higher than 5 million cells.

In some embodiments, the obtaining the cellular constituent count data structure 160 comprises performing a dimension reduction (e.g., using any of the methods and/or embodiments described herein in the sections entitled, “Cellular constituent modules” and “Latent representations,” above). In some embodiments, the obtaining the cellular constituent count data structure 160 comprises obtaining a representation of the plurality of cellular constituents, such that the cellular constituent count data structure is dimensioned by (i) the second plurality of cells and (ii) the plurality of cellular constituents or the representation thereof.

Activation Data Structures.

Referring to Block 210, the method further comprises forming an activation data structure by combining the cellular constituent count data structure and the latent representation using the plurality of cellular constituents or the representation thereof as a common dimension, where the activation data structure comprises, for each cellular constituent module in the plurality of cellular constituent modules, for each cell in the second plurality of cells, a respective activation weight.

To accomplish this, in some embodiments, a count matrix (e.g., the cellular constituent count data structure 160) and a cellular constituent modules matrix (e.g., the latent representation 118) are multiplied together, such that the weights of the modules matrix are multiplied by the normalized counts of the count matrix. Generally, two matrices can be multiplied together by a common dimension (e.g., the x-axis of a first matrix and the y-axis of a second matrix). Matrix multiplication of the first and second matrices by their common dimension yields a third matrix of auxiliary data that can be applied, alternatively or in addition to the first matrix and/or the second matrix, to an untrained or partially trained model.

Thus, in some such embodiments, the count matrix has the dimensions n_cells×n_genes, and the latent representation has the dimensions n_genes×n_modules, where n_cells is the number of cells in the second plurality of cells, n_genes is the number of cellular constituents (e.g., genes), or the representation thereof, in the plurality of cellular constituents, and n_modules is the number of modules in the plurality of cellular constituent modules. This maps the abundances of the cellular constituents in the count matrix into a space in which each cell (e.g., corresponding to one or more covariates of interest) is characterized by its module activations, and in which the resulting matrix representation (e.g., the activation data structure 170) has dimensions n_cells×n_modules (e.g., after multiplying by a common dimension of n_genes).

Accordingly, FIG. 1D illustrates a resulting activation data structure 170 having dimensions n_cells×n_modules. The activation data structure includes, for each cell in the second plurality of cells (e.g., G cells), for each respective cellular constituent module in the plurality of cellular constituent modules 172 (e.g., K cellular constituent modules), a corresponding activation weight 174 that indicates the activations of the respective module in the respective cell.

Combination of the latent representation 118 and the cellular constituent count data structure 160 using, e.g., matrix multiplication, and the resulting activation data structure in matrix form, are collectively illustrated in FIG. 5, with further reference to FIGS. 1C-D. The latent representation (illustrated in the top panel of FIG. 5) has the dimensions Z×K, where Z is the number of cellular constituents or the representation thereof and K is the number of cellular constituent modules. The cellular constituent count data structure (illustrated in the lower left panel) has the dimensions G×Z, where G is the number of cells in the second plurality of cells and, as for the latent representation, Z is the number of cellular constituents or the representation thereof. Combination by matrix multiplication, using Z (the number of cellular constituents or the representation thereof) as a common dimension, generates the resulting activation data structure having dimensions G×K. Each entry for each respective column in each respective row is an activation weight indicating the activation of each respective cellular constituent module in the respective cell in the second plurality of cells corresponding to the respective column. Thus, as illustrated in FIG. 5, the counts corresponding to module 1 includes activation weight₁₋₁ corresponding to cell 1, activation weight_{1-G} corresponding to cell G, and so on.

In some embodiments, the plurality of activation weights in the activation data structure comprises differential module activations.

In some embodiments, differential module activations (e.g., differential activation weights of a respective module between cells in the second plurality of cells in the activation data structure) are obtained by computing v-scores using the function (mu_1−mu_2)/(var_1+var_2)^{-0.5}, where mu_i denotes means of module activations across cells with a respective condition i (e.g., covariate i), and var_i denotes the variance of module activation in condition i. V-scores can be described as t-scores that are not normalized by the number of cells in the denominator.

The activation data structure therefore indicates activations (e.g., the level or degree of activation) of a respective cellular constituent module corresponding to (e.g., correlated to and/or in response to) one or more covariates in the plurality of covariates represented by the second plurality of cells. Thus, in some embodiments where the plurality of covariates represented by the second plurality of cells includes a respective perturbagen (e.g., a compound to which one or more cells are exposed), the activation data structure will include a respective activation weight, for each respective cellular constituent module in the plurality of cellular constituent modules, indicating the activation (e.g., inducement and/or differential expression) of the respective cellular constituent module, correlating to and/or in response to treatment with the respective compound.

Models.

Referring to Block 212, the method further includes training a model using, for each respective covariate in the plurality of covariates, a difference between (i) a calculated activation against each cellular constituent module represented by the model upon input of a representation of the respective covariate into the model and (ii) actual activation against each cellular constituent module represented by the model. In some embodiments, the model is an ensemble model that includes a component model for each cellular constituent model in the plurality of cellular constituent models. The training adjusts a plurality of covariate weights associated with the model responsive to the difference, where the plurality of covariate weights comprises, for each respective cellular constituent module in the plurality of cellular constituent modules, for each respective covariate, a corresponding weight indicating whether the respective covariate correlates, across the second plurality of cells, with the respective cellular constituent module.

In some embodiments, input to the model includes a representation of a respective covariate, and the activation represented by the respective covariate serves as a label (e.g., categorical value indication absence or presence of a response to the covariate, or a value drawn from a continuous scale indicating the extent of response to a covariate) for training a multi-task model to identify correlations between modules and covariates of interest. In this way, it is possible to distinguish between core features of the input (e.g., genes, transformation of genes, and/or cellular constituent modules) and covariates of interest (e.g., cell batch, cell donor, cell type, cell line, and/or disease status). In some embodiments, a respective covariate in the plurality of covariates comprises cell batch and the representation of the respective covariate that is inputted into the model is a cell batch identification. In some embodiments a respective covariate in the plurality of covariates that is inputted into the model comprises cell donor and the representation of the respective covariate is an identification of the cell donor or a characteristic of the cell donor. In some embodiments, a respective covariate in the plurality of covariates comprises cell type and the representation of the respective covariate that is inputted into the model is a cell type identification. In some embodiments a respective covariate in the plurality of covariates comprises disease status and the representation of the respective covariate that is inputted into the model is an indication of absence or presence of the disease. In some embodiments a respective covariate in the plurality of covariates comprises exposure to a compound and the representation of the respective covariate that is inputted into the model is a fingerprint of the compound. In some such embodiments the method further comprises generating the fingerprint from a chemical structure of the compound using Daylight, BCI, ECFP4, EcFC, MDL, TTFP, UNITY 2D, RNNS2S, GraphConv, fingerprint SMILES Transformer, RNNS2S, or GraphConv. In some embodiments, the representation of the respective covariate further comprises a duration of time the respective covariate was incubated with the respective cell. In some embodiments, the representation of the respective covariate further comprises a concentration of the respective covariate used to incubate the respective cell.

In some embodiments, an output of the model includes a cellular constituent module knowledge construct 124 (e.g., a covariate weight matrix), as illustrated in FIG. 1E. FIG. 1E illustrates an example cellular constituent module knowledge construct including a plurality of covariate weights (e.g., knowledge term weight 130) indicating whether each respective covariate in a plurality of covariates (e.g., knowledge term identity 128) correlates, across the second plurality of cells, with each respective cellular constituent module in the plurality of cellular constituent modules 126. In some embodiments, each respective cellular constituent module is associated (e.g., correlated) with a different subset of covariates. FIG. 1E shows, for instance, 3 example modules, each module correlated with a different subset of knowledge terms (e.g., P, Q, and S).

In some embodiments, the model is an autoencoder, a sparse autoencoder, and/or a sparse multi-readout, knowledge-coupled autoencoder. In some embodiments, the model is a semi-supervised model. In some embodiments, the model is a one-layer neural network (e.g., a SoftMax and/or logistic regression). In some embodiments, the model is a one-dimensional Huber Outlier Regressor model. In some embodiments, the model comprises 100 or more parameters, 1000 or more parameters, 10,000 or more parameters, 100,000 or more parameters, or 1×10⁶ or more parameters.

In an example embodiment, the method comprises using a sparse autoencoder to obtain both the latent representation 118 and the cellular constituent module knowledge construct 124. In some embodiments, the autoencoder comprises a plurality of layers, where the first layer is used to obtain the latent representation 118 and the second layer is used to obtain the cellular constituent module knowledge construct 124 (e.g., a covariate weight matrix).

In some embodiments, the model is any of the models disclosed herein (see, for example, Definitions: Models).

For instance, in some embodiments, the model is a neural network comprising one or more inputs, a corresponding first hidden layer comprising a corresponding plurality of hidden neurons, where each hidden neuron in the corresponding plurality of hidden neurons (i) is fully connected to each input in the plurality of inputs, (ii) is associated with a first activation function type, and (iii) is associated with a corresponding parameter (e.g., weight) in a plurality of parameters for the neural network, and one or more corresponding neural network outputs, where each respective neural network output in the corresponding one or more neural network outputs (i) directly or indirectly receives, as input, an output of each hidden neuron in the corresponding plurality of hidden neurons, and (ii) is associated with a second activation function type. In some such embodiments, the neural network is a fully connected network.

In some embodiments, the neural network comprises a plurality of hidden layers. As described above, hidden layers are located between input and output layers (e.g., to capture additional complexity). In some embodiments, where there is a plurality of hidden layers, each hidden layer may have a same respective number of neurons.

In some embodiments, each hidden neuron (e.g., in a respective hidden layer in a neural network) is associated with an activation function that performs a function on the input data (e.g., a linear or non-linear function). Generally, the purpose of the activation function is to introduce nonlinearity into the data such that the neural network is trained on representations of the original data and can subsequently “fit” or generate additional representations of new (e.g., previously unseen) data. Selection of activation functions (e.g., a first and/or a second activation function) is dependent on the use case of the neural network, as certain activation functions can lead to saturation at the extreme ends of a dataset (e.g., tan h and/or sigmoid functions). For instance, in some embodiments, an activation function (e.g., a first and/or a second activation function) is selected from any suitable activation functions known in the art.

In some embodiments, each hidden neuron is further associated with a parameter (e.g., a weight and/or a bias value) that contributes to the output of the neural network, determined based on the activation function. In some embodiments, the hidden neuron is initialized with arbitrary parameters (e.g., randomized weights). In some alternative embodiments, the hidden neuron is initialized with a predetermined set of parameters.

In some embodiments, the plurality of hidden neurons in a neural network (e.g., across one or more hidden layers) is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, or at least 500 neurons. In some embodiments, the plurality of hidden neurons is at least 100, at least 500, at least 800, at least 1000, at least 2000, at least 3000, at least 4000, at least 5000, at least 6000, at least 7000, at least 8000, at least 9000, at least 10,000, at least 15,000, at least 20,000, or at least 30,000 neurons. In some embodiments, the plurality of hidden neurons is no more than 30,000, no more than 20,000, no more than 15,000, no more than 10,000, no more than 9000, no more than 8000, no more than 7000, no more than 6000, no more than 5000, no more than 4000, no more than 3000, no more than 2000, no more than 1000, no more than 900, no more than 800, no more than 700, no more than 600, no more than 500, no more than 400, no more than 300, no more than 200, no more than 100, or no more than 50 neurons. In some embodiments, the plurality of hidden neurons is from 2 to 20, from 2 to 200, from 2 to 1000, from 10 to 50, from 10 to 200, from 20 to 500, from 100 to 800, from 50 to 1000, from 500 to 2000, from 1000 to 5000, from 5000 to 10,000, from 10,000 to 15,000, from 15,000 to 20,000, or from 20,000 to 30,000 neurons. In some embodiments, the plurality of hidden neurons falls within another range starting no lower than 2 neurons and ending no higher than 30,000 neurons.

In some embodiments, the neural network comprises from 1 to 50 hidden layers. In some embodiments, the neural network comprises from 1 to 20 hidden layers. In some embodiments, the neural network comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, or at least 100 hidden layers. In some embodiments, the neural network comprises no more than 100, no more than 90, no more than 80, no more than 70, no more than 60, no more than 50, no more than 40, no more than 30, no more than 20, no more than 10, no more than 9, no more than 8, no more than 7, no more than 6, or no more than 5 hidden layers. In some embodiments, the neural network comprises from 1 to 5, from 1 to 10, from 1 to 20, from 10 to 50, from 2 to 80, from 5 to 100, from 10 to 100, from 50 to 100, or from 3 to 30 hidden layers. In some embodiments, the neural network comprises a plurality of hidden layers that falls within another range starting no lower than 1 layer and ending no higher than 100 layers.

In some embodiments, the neural network comprises is a shallow neural network. A shallow neural network refers to a neural network with a small number of hidden layers. In some embodiments, such neural network architectures improve the efficiency of neural network training and conserve computational power due to the reduced number of layers involved in the training. In some embodiments, the neural network comprises has only one hidden layer.

In some embodiments, the model comprises a plurality of parameters (e.g., weights and/or hyperparameters). In some embodiments, the plurality of parameters for the model comprises at least 10, at least 50, at least 100, at least 500, at least 1000, at least 2000, at least 5000, at least 10,000, at least 20,000, at least 50,000, at least 100,000, at least 200,000, at least 500,000, at least 1 million, at least 2 million, at least 3 million, at least 4 million or at least 5 million parameters. In some embodiments, the plurality of parameters for the model comprises no more than 8 million, no more than 5 million, no more than 4 million, no more than 1 million, no more than 500,000, no more than 100,000, no more than 50,000, no more than 10,000, no more than 5000, no more than 1000, or no more than 500 parameters. In some embodiments, the plurality of parameters for the model comprises from 10 to 5000, from 500 to 10,000, from 10,000 to 500,000, from 20,000 to 1 million, or from 1 million to 5 million parameters. In some embodiments, the plurality of parameters for the model falls within another range starting no lower than 10 parameters and ending no higher than 8 million parameters.

In some embodiments, the model is associated with one or more activation functions. In some embodiments, an activation function in the one or more activation functions is tan h, sigmoid, softmax, Gaussian, Boltzmann-weighted averaging, absolute value, linear, rectified linear unit (ReLU), bounded rectified linear, soft rectified linear, parameterized rectified linear, average, max, min, sign, square, square root, multiquadric, inverse quadratic, inverse multiquadric, polyharmonic spline, swish, mish, Gaussian error linear unit (GeLU), and/or thin plate spline.

In some embodiments, the training of the model is further characterized by one or more hyperparameters (e.g., one or more values that may be tuned during training). In some embodiments, the hyperparameter values are tuned (e.g., adjusted) during training. In some embodiments, the hyperparameter values are determined based on the specific elements of the training dataset and/or one or more inputs (e.g., cells, cellular constituent modules, covariates, etc.). In some embodiments, the hyperparameter values are determined using experimental optimization. In some embodiments, the hyperparameter values are determined using a hyperparameter sweep. In some embodiments, the hyperparameter values are assigned based on prior template or default values.

In some embodiments, a respective hyperparameter of the one or more hyperparameters comprises a learning rate. In some embodiments, the learning rate is at least 0.0001, at least 0.0005, at least 0.001, at least 0.005, at least 0.01, at least 0.05, at least 0.1, at least 0.2, at least 0.3, at least 0.4, at least 0.5, at least 0.6, at least 0.7, at least 0.8, at least 0.9, or at least 1. In some embodiments, the learning rate is no more than 1, no more than 0.9, no more than 0.8, no more than 0.7, no more than 0.6, no more than 0.5, no more than 0.4, no more than 0.3, no more than 0.2, no more than 0.1 no more than 0.05, no more than 0.01, or less. In some embodiments, the learning rate is from 0.0001 to 0.01, from 0.001 to 0.5, from 0.001 to 0.01, from 0.005 to 0.8, or from 0.005 to 1. In some embodiments, the learning rate falls within another range starting no lower than 0.0001 and ending no higher than 1. In some embodiments, the one or more hyperparameters further include regularization strength (e.g., L2 weight penalty, dropout rate, etc.). For instance, in some embodiments, the model (e.g., a neural network) is trained using a regularization on a corresponding parameter (e.g., weight) of each hidden neuron in the plurality of hidden neurons. In some embodiments, the regularization includes an L1 or L2 penalty.

In some embodiments, a respective hyperparameter of the one or more hyperparameters is a loss function. In some embodiments, the loss function is mean square error, flattened mean square error, quadratic loss, mean absolute error, mean bias error, hinge, multi-class support vector machine, and/or cross-entropy. In some embodiments, the loss function is a gradient descent algorithm and/or a minimization function.

Model Training.

Generally, training a model (e.g., a neural network) comprises updating the plurality of parameters (e.g., weights) for the respective model through backpropagation (e.g., gradient descent). First, a forward propagation is performed, in which input data (e.g., an activation data structure comprising activation weights for each respective cell in the second plurality of cells, for each respective cellular constituent module in a plurality of modules, and a corresponding plurality of covariates represented by the plurality of cells) is accepted into the neural network, and an output is calculated based on the selected activation function and an initial set of parameters (e.g., weights and/or hyperparameters). In some embodiments, parameters (e.g., weights and/or hyperparameters) are randomly assigned (e.g., initialized) for an untrained or partially trained model. In some embodiments, parameters are transferred from a previously saved plurality of parameters or from a pre-trained model (e.g., by transfer learning).

A backward pass is then performed by calculating an error gradient for each respective parameter corresponding to each respective unit in each layer, where the error for each parameter is determined by calculating a loss (e.g., error) based on the network output (e.g., the predicted absence or presence of each covariate in each cellular constituent module) and the input data (e.g., the expected value or true labels; the actual absence or presence of each covariate in each cellular constituent module). Parameters (e.g., weights) are then updated by adjusting the value based on the calculated loss, thereby training the model.

For example, in some general embodiments of machine learning, backpropagation is a method of training a network with hidden layers comprising a plurality of weights (e.g., embeddings). The output of an untrained model (e.g., the predicted absence or presence of covariates) is first generated using a set of arbitrarily selected initial weights. The output is then compared with the original input (e.g., the actual absence or presence of covariates) by evaluating an error function to compute an error (e.g., using a loss function). The weights are then updated such that the error is minimized (e.g., according to the loss function). In some embodiments, any one of a variety of backpropagation algorithms and/or methods are used to update the plurality of weights, as will be apparent to one skilled in the art.

In some embodiments, the loss function is mean square error, quadratic loss, mean absolute error, mean bias error, hinge, multi-class support vector machine, and/or cross-entropy. In some embodiments, training an untrained or partially trained model comprises computing an error in accordance with a gradient descent algorithm and/or a minimization function. In some embodiments, training an untrained or partially trained model comprises computing a plurality of errors using a plurality of loss functions. In some embodiments, each loss function in a plurality of loss functions receives a same or a different weighting factor.

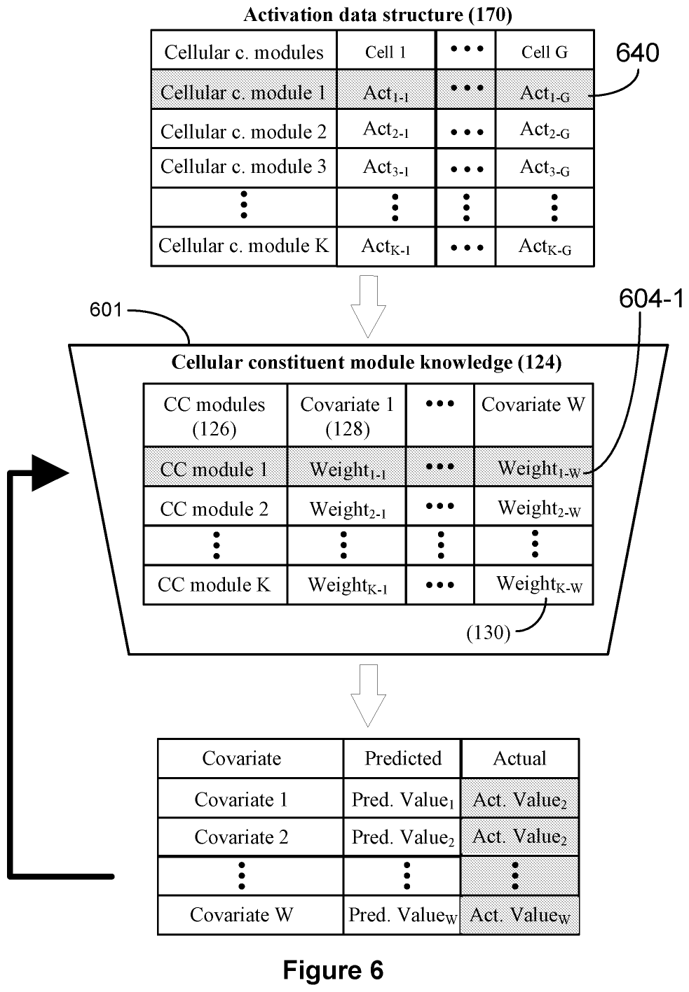

Turning to FIG. 6, in some instances, each row of activation data structure 170 (from FIG. 5 and now at the top of FIG. 6) serves as training data for a different model 601. For instance, consider the case where model 601 includes the weights of row 604-1 (Weight₁₋₁ through Weight_{1-W}) to represent the extent to which covariates 1 through W respectively activate cellular constituent module 1. This model 601 is trained on the elements of row 640 of activation data structure 170, which provide the extent to which each of the covariates 1, . . . , G activate cellular constituent module 1. In this training, first a representation of the covariate associated with cell 1 is inputted into the model 601. Responsive to this input, the model 601 for cellular constituent module 1 outputs an activation value, termed Pred. Value₁ in the nomenclature of FIG. 6. This output activation value is compared to the actual activation value for the covariate associated with cell 1, which is Act₁₋₁ of activation data structure 170. Next, a representation of the covariate associated with cell 2 is inputted into the model 601. In response to this input, the model outputs an activation value (Pred. Value₂). This output activation value is compared to the actual activation value for the covariate associated with cell 2 of row 640, which is Act₁₋₂ of activation data structure 170. This process proceeds through cell G. A representation of the covariate associated with cell G is inputted into the model 601. In response to this, the model will output an activation value (Pred. Value_{G}). This output activation value is compared to the actual activation value for cell G, which is Act_{1-G} of row 640 of activation data structure 170. In this example, W and G have the same value. In this way, there is a resulting prediction (Pred. Value) for each covariate in the plurality of covariates used to derive the activation data structure as outlined in FIG. 5 for cellular constituent module 1. The above-described calculated predictions (of the activation value) are compared to the above-described actual activation values for each of these covariates and the differences between the predicted and actual activation values are used to further train the model 601 using back-propagation and related model refinement techniques.

In some embodiments, the error function is used to update one or more parameters (e.g., weights) in a model (e.g., a neural network) by adjusting the value of the one or more parameters by an amount proportional to the calculated loss, thereby training the model. In some embodiments, the amount by which the parameters are adjusted is metered by a learning rate hyperparameter that dictates the degree or severity to which parameters are updated (e.g., smaller or larger adjustments). Thus, in some embodiments, the training updates all or a subset of the plurality of parameters based on a learning rate. In some embodiments, the learning rate is a differential learning rate.

In some embodiments, training a model (e.g., a neural network) further uses a regularization on the corresponding parameter of each hidden neuron in the corresponding plurality of hidden neurons. For example, in some embodiments, a regularization is performed by adding a penalty to the loss function, where the penalty is proportional to the values of the parameters in the neural network. Generally, regularization reduces the complexity of the model by adding a penalty to one or more parameters to decrease the importance of the respective hidden neurons associated with those parameters. Such practice can result in a more generalized model and reduce overfitting of the data. In some embodiments, the regularization includes an L1 or L2 penalty. For example, in some preferred embodiments, the regularization includes an L2 penalty on lower and upper parameters. In some embodiments, the regularization comprises spatial regularization (e.g., determined based on a priori and/or experimental knowledge) or dropout regularization. In some embodiments, the regularization comprises penalties that are independently optimized.

In some embodiments, the training process including the adjusting the plurality of covariate weights associated with the model (e.g., responsive to the difference between the predicted and actual labels), is repeated for each training instance in a plurality of training instances.

In some embodiments, the plurality of training instances comprises at least 3, at least 4, at least 5, at least 6, at least 7, at least 8, at least 9, at least 10, at least 50, at least 100, at least 500, at least 1000, at least 2000, at least 3000, at least 4000, at least 5000, or at least 7500 training instances. In some embodiments, the plurality of training instances comprises no more than 10,000, no more than 5000, no more than 1000, no more than 500, no more than 100, or no more than 50 training instances. In some embodiments, the plurality of training instances comprises from 3 to 10, from 5 to 100, from 100 to 5000, or from 1000 to 10,000 training instances. In some embodiments, the plurality of training instances falls within another range starting no lower than 3 training instances and ending no higher than 10,000 training instances.

In some such embodiments, the training includes repeating the adjustment of the parameters of the model (e.g., via backpropagation) over a plurality of training instances, therefore increasing the model's accuracy in indicating whether a respective covariate correlates with a respective cellular constituent module.

In some embodiments, the training comprises transfer learning. Transfer learning is further described, for example, in the Definitions section (see, “Untrained models,” above).

In some embodiments, training an untrained or partially trained model forms a trained model following a first evaluation of an error function. In some such embodiments, the trained model is formed following a first updating of one or more parameters based on a first evaluation of an error function. In some alternative embodiments, the trained model is formed following at least 1, at least 2, at least 3, at least 4, at least 5, at least 6, at least 7, at least 8, at least 9, at least 10, at least 20, at least 30, at least 40, at least 50, at least 100, at least 500, at least 1000, at least 10,000, at least 50,000, at least 100,000, at least 200,000, at least 500,000, or at least 1 million evaluations of an error function. In some such embodiments, the trained model is formed following at least 1, at least 2, at least 3, at least 4, at least 5, at least 6, at least 7, at least 8, at least 9, at least 10, at least 20, at least 30, at least 40, at least 50, at least 100, at least 500, at least 1000, at least 10,000, at least 50,000, at least 100,000, at least 200,000, at least 500,000, or at least 1 million updatings of one or more parameters based on the at least 1, at least 2, at least 3, at least 4, at least 5, at least 6, at least 7, at least 8, at least 9, at least 10, at least 20, at least 30, at least 40, at least 50, at least 100, at least 500, at least 1000, at least 10,000, at least 50,000, at least 100,000, at least 200,000, at least 500,000, or at least 1 million evaluations of an error function.

In some embodiments, the trained model is formed when the model satisfies a minimum performance requirement. For example, in some embodiments, the trained model is formed when the error calculated for the trained model, following an evaluation of an error function (e.g., a difference between a predicted and an actual absence or presence of each covariate in each cellular constituent module represented in the activation data structure) satisfies an error threshold. In some embodiments, the error calculated by the error function satisfies an error threshold when the error is less than 20 percent, less than 18 percent, less than 15 percent, less than 10 percent, less than 5 percent, or less than 3 percent.

In an example embodiment, the training the model is performed using a categorical cross-entropy loss in a multi-task formulation, in which each covariate in the plurality of covariates corresponds to a cost function in plurality of cost functions and each respective cost function in the plurality of cost functions has a common weighting factor.

In some embodiments, training a model provides weights for each covariate and hence reveals modules that inform covariates possibly informative of the cellular process of interest (e.g., disease status over donor identity). The dimensions of the covariate weight matrix are n_covariates×n_modules, where n_covariates is the number of covariates in the plurality of covariates represented by the second plurality of cells and n_modules is the number of modules in the plurality of cellular constituent modules. Thus, each weight indicates the degree to which each covariate associates with the activation of a module. These modules can be used in downstream applications to elucidate the molecular mechanisms underlying the cellular process of interest, such as drug discovery.

In some embodiments, the training the model comprises obtaining a plurality of covariate weights that can be used for cross-system identification of modules (e.g., where modules are shared between systems and/or orthogonal across systems). Typically, cellular constituent modules (e.g., gene modules) decompose disease-induced and perturbation-induced variation into shared and orthogonal biological modules across systems. To systematically arrive at critical factors and cellular programs that translate across a wide array of in vitro and in vivo sources (e.g., pre-clinical models and/or patients), a cross-system modeling approach can be used to analyze cellular processes of interest at varying levels of resolution (e.g., the gene, gene module, and/or compound level). In some embodiments, a cross-system model is used to identify shared and/or orthogonal cellular constituent modules across any one or more of: multiple disease-relevant and/or perturbational datasets; multiple cell types, cell states, and/or cell trajectories; multiple experiment types (e.g., in vitro and/or in vivo); multiple tissue, subject, and/or species; and/or multiple facilities or sample collection points (e.g., laboratories and/or clinics).

In an example implementation of a cross-system approach, two complex cell systems are considered (e.g., an in vitro model of hematopoiesis and a mouse model), from which cellular constituent data (e.g., scRNA-seq of the bone marrow containing all lineages of the hematopoietic developmental processes) is obtained. The cellular constituent data (e.g., scRNA-seq data) sampled from these two systems is transformed into module activation space.

For given comparisons of interest (e.g., differentiated megakaryocytes versus un-differentiated megakaryocytes, and/or healthy donors versus diseased donors), differential module activations are determined by computing v-scores (mu_1−mu_2)/(var_1+var_2)^{-0.5}, where mu_i denotes means of module activations across cells in the condition i, and var_i denotes the variance of module activation in condition i. In some instances, v-scores can be represented as t-scores that are not normalized by the number of cells in the denominator.

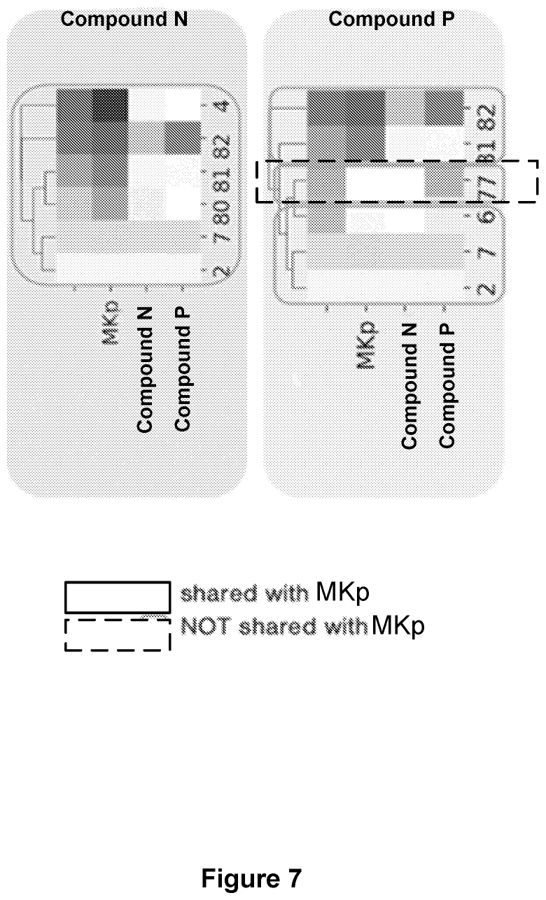

Using the obtained t-scores, in some embodiments, a model (e.g., a 1-dimensional Huber Outlier Regressor model) is trained by defining module activations from one system as the response variable y and the module activations from the other system as the predictor variable x. After training, the model provides a one-dimensional regression relationship that estimates how well module activations in system x explain module activations in system y. For instance, in particular, the Huber regressor identifies outlier modules that are activated in a highly orthogonal fashion. An example comparison of interest is illustrated in Example 1 with reference to FIG. 7, below.

Pruning.

Referring to Block 214, the method further includes identifying, using the plurality of covariate weights upon training the model, one or more cellular constituent modules in the plurality of cellular constituent modules that is associated with one or more covariates in the plurality of covariates, thereby associating a plurality of cellular constituents with the cellular process of interest.

In some embodiments, the method further comprises using the identity of each cellular constituent in a cellular constituent module in the plurality of cellular constituent modules to associate the cellular constituent module with a known cellular pathway, a biological process, a transcription factor, a cell receptor, or a kinase using a contextualization algorithm. In some embodiments, the contextualization algorithm is a gene set enrichment analysis algorithm.

In some embodiments, the method further comprises, for each respective cellular constituent module in the plurality of cellular constituent modules, using the identity of each cellular constituent in the respective cellular constituent module to associate the respective cellular constituent module with a known cellular pathway, a biological process, a transcription factor, a cell receptor, or a kinase using a contextualization algorithm. In some embodiments, the contextualization algorithm is a gene set enrichment analysis algorithm.

In some embodiments, the contextualization algorithm is any contextualization algorithm and/or any annotation method disclosed herein, including those further described in the section entitled, “Contextualization,” below.

In some embodiments, the method further comprises pruning the activation data structure by removing from the activation data structure one or more cellular constituent modules that fail to associate with a known cellular pathway, a biological process, a transcription factor, a cell receptor, or a kinase that is implicated in the cellular process of interest. For instance, in some embodiments, the pruning allows for the prioritization of cellular constituent modules that are most likely to be associated with a cellular process of interest (e.g., prioritization of disease-critical and/or process-critical gene modules). In some embodiments, the pruning allows for the prioritization of candidate targets for drug discovery.

Contextualization.

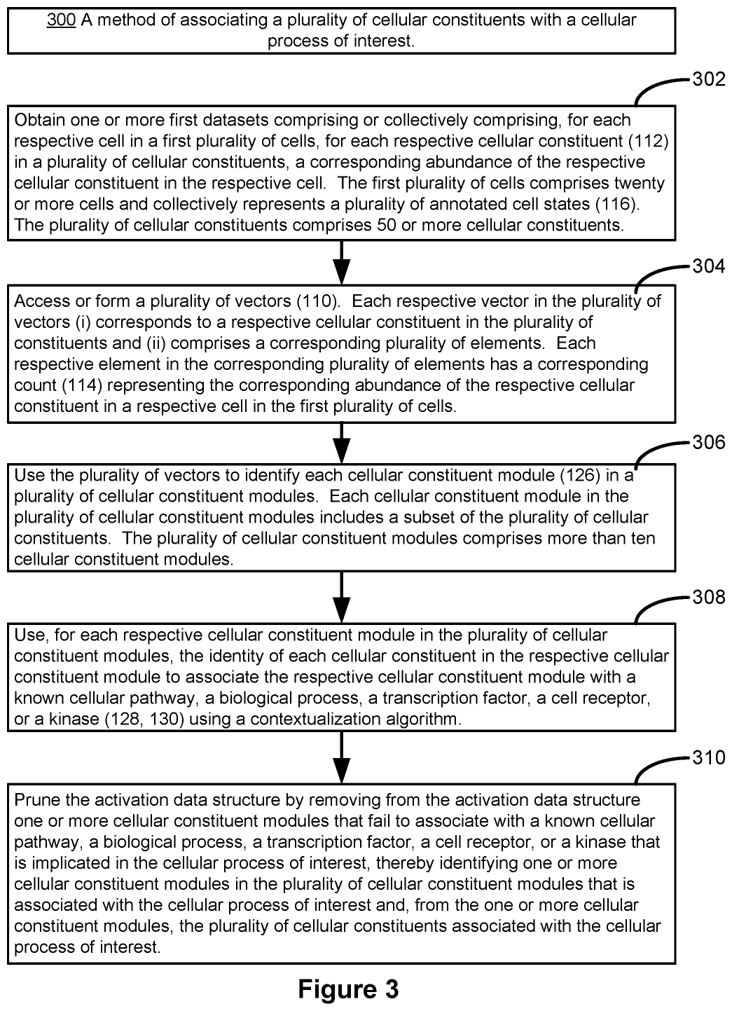

Referring to FIG. 3, another aspect of the present disclosure provides a method 300 of associating a plurality of cellular constituents with a cellular process of interest. The method 300 includes a contextualization that is used to infer knowledge for each cellular constituent module. For instance, in some embodiments, the contextualization is used alternatively, or in addition, to a model for obtaining a latent representation and a covariate weight matrix.

Referring to Block 302, the method comprises, at a computer system comprising a memory and one or more processors obtaining one or more first datasets in electronic form, the one or more first datasets comprising or collectively comprising, for each respective cell in a first plurality of cells, where the first plurality of cells comprises, e.g., twenty or more cells and collectively represents a plurality of annotated cell states, for each respective cellular constituent in a plurality of cellular constituents, where the plurality of cellular constituents comprises, e.g., 50 or more cellular constituents, a corresponding abundance of the respective cellular constituent in the respective cell.

Referring to Block 304, the method further includes accessing or forming a plurality of vectors, each respective vector in the plurality of vectors (i) corresponding to a respective cellular constituent in the plurality of constituents and (ii) comprising a corresponding plurality of elements, each respective element in the corresponding plurality of elements having a corresponding count representing the corresponding abundance of the respective cellular constituent in a respective cell in the first plurality of cells.

It is to be understood that any of the methods and embodiments for cells in the first plurality of cells, including but not limited to type, number, source, cellular processes, and/or annotated cell states, disclosed herein in the above section entitled, “Cell states and processes,” are contemplated with respect to the method 300, and/or any substitutions, modifications, additions, deletions, and/or combinations thereof, as will be apparent to one skilled in the art. It is to be further understood that any of the methods and embodiments for cellular constituents in the plurality of cellular constituents, including but not limited to type, number, methods of measurement, preprocessing, filtering, and/or transformations, disclosed herein in the above section entitled, “Cellular constituents,” and/or any substitutions, modifications, additions, deletions, and/or combinations thereof, are contemplated with respect to the method 300. For instance, the plurality of cellular constituent vectors can be represented as the count matrix 110 illustrated in FIG. 1B and FIG. 4 (top panel). The count matrix 110 includes a corresponding count 114 for each corresponding abundance of each cellular constituent 112, for each respective cell in the first plurality of cells representing a plurality of annotated cell states 116.

Referring to Block 306, the method further comprises using the plurality of vectors to identify each cellular constituent module in a plurality of cellular constituent modules, each cellular constituent module in the plurality of cellular constituent modules including a subset of the plurality of cellular constituents (e.g., where the plurality of cellular constituent modules comprises more than ten cellular constituent modules). It is to be understood that any of the methods and embodiments for cellular constituent modules, including but not limited to type, number, membership, clustering algorithms and/or partitioning methods, dimension reduction and/or latent representations, disclosed herein in the above sections entitled, “Cellular constituent modules” and “Latent representations,” are contemplated with respect to the method 300, and/or any substitutions, modifications, additions, deletions, and/or combinations thereof, as will be apparent to one skilled in the art.

In some embodiments, the method further comprises determining a knowledge contextualization for each respective cellular constituent module in the plurality of cellular constituent modules. In some embodiments, the determining a knowledge contextualization comprises obtaining, for each respective cellular constituent module in the plurality of cellular constituent modules, one or more knowledge terms and/or a corresponding one or more contextualization scores (e.g., knowledge term weights).

Thus, referring to Block 308, the method includes, for each respective cellular constituent module in the plurality of cellular constituent modules, using the identity of each cellular constituent in the respective cellular constituent module to associate the respective cellular constituent module with a known cellular pathway, a biological process, a transcription factor, a cell receptor, or a kinase using a contextualization algorithm.

In some embodiments, knowledge contextualization refers to the incorporation of external knowledge sources to add biological significance to one or more cellular constituent modules, which can be used to obtain meaningful interpretations from the dataset. In some embodiments, knowledge contextualization is data-driven (e.g., using statistical analyses of large datasets such as genome-wide association studies (GWAS) and/or gene set enrichment analysis (GSEA)). In some embodiments, knowledge contextualization is theory-driven (e.g., using proposed or experimentally determined relationships between a small number of cellular constituents and cellular processes of interest to establish candidate modules for further validation). While data-driven and theory-driven approaches have unique advantages and limitations, the use of one or both methods to annotate and contextualize cellular constituent modules in accordance with embodiments of the present disclosure can improve the interpretation and prioritization of modules for deeper understanding of the cellular processes of interest. For example, large datasets are amenable to robust statistical analyses but can be subject to noise and oversensitivity to outliers that exhibit high levels of variation but are not biologically meaningful. In some embodiments, inclusion of theory-driven annotations and other knowledge terms removes noise and focuses the prioritization of cellular constituent modules on those that are most likely to have biological relevance.

In some embodiments, the contextualization algorithm is a gene set enrichment analysis (GSEA) algorithm. In some embodiments, each cellular constituent module in the plurality of cellular constituent modules includes a GSEA score indicating the differential enrichment of the cellular constituents within each module across the plurality of modules. Methods for obtaining GSEA scores are described, for example, in Subramanian et al., 2005, PNAS 102, 15545-15550 and Liberzon et al., 2015, Cell Systems 23; 1(6): 417-425, each of which is hereby incorporated herein by reference in its entirety.

In some embodiments, the contextualization algorithm is a genome-wide association study (GWAS). GWAS is a data-driven approach that can be used to identify biomarkers (e.g., SNPs) that are enriched in individuals characterized by one or more phenotypes of interest (e.g., diseases).

In some embodiments, the contextualization algorithm comprises obtaining a combined score obtained, at least in part, from GSEA scores and GWAS scores. In some embodiments, the contextualization algorithm comprises obtaining a combined score obtained for a plurality of cellular constituents (e.g., a polygenic score). Methods for obtaining GSEA scores, GWAS scores, and polygenic scores are further described in, for example, Elam et al., 2019, Translational Psychiatry 9:212; doi: 10.1038/s41398-019-0513-7, which is hereby incorporated herein by reference in its entirety.

In some embodiments, the contextualization algorithm assigns, for each respective cellular constituent module in the plurality of cellular constituent modules, for each respective knowledge term in a plurality of knowledge terms, a corresponding contextualization score. Thus, the plurality of contextualization scores for each respective cellular constituent module in the plurality of cellular constituent modules can be represented as the cellular constituent module knowledge construct 124 illustrated in FIG. 1E. The knowledge construct 124 includes a corresponding knowledge term 128 and a corresponding knowledge term weight (e.g., score) 130 for each respective cellular constituent module in a plurality of cellular constituent modules 126.

In some embodiments, the contextualization algorithm assigns, to a respective cellular constituent module in the plurality of cellular constituent modules, at least at least 3, at least 5, at least 10, at least 15, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, or at least 3000 knowledge terms. In some embodiments, the contextualization algorithm assigns, to a respective cellular constituent module in the plurality of cellular constituent modules, no more than 5000, no more than 1000, no more than 500, no more than 200, no more than 100, no more than 50, or no more than 20 knowledge terms. In some embodiments, the contextualization algorithm assigns, to a respective cellular constituent module in the plurality of cellular constituent modules, from 3 to 10, from 10 to 50, from 20 to 500, from 200 to 1000, or from 1000 to 5000 knowledge terms. In some embodiments, the contextualization algorithm assigns, to a respective cellular constituent module in the plurality of cellular constituent modules, a plurality of knowledge terms that falls within another range starting no lower than 3 knowledge terms and ending no higher than 5000 knowledge terms.

In some embodiments, the contextualization score is a weight. In some embodiments, the contextualization score is a binary label (e.g., absence or presence of the knowledge term associated with the respective cellular constituent module). In some embodiments, the contextualization score is a correlation coefficient. In some embodiments, the contextualization score is a GSEA score, a GWAS score, and/or a polygenic risk score. In some embodiments, the contextualization score comprises a measure of central tendency (e.g., a mean, median, and/or mode) across the subset of cellular constituents for a respective cellular constituent module. In some embodiments, the contextualization score comprises a measure of dispersion (e.g., a standard deviation, standard error, and/or confidence interval) across the subset of cellular constituents for a respective cellular constituent module. In some embodiments, the contextualization score comprises a measure of confidence (e.g., a p-value, false discovery rate (FDR), and/or q-value). In some embodiments, the contextualization score is a GSEA p-value.

In some embodiments, each respective knowledge term associated with a respective cellular constituent module is ranked according to the corresponding contextualization score (e.g., weight) for the respective knowledge term.

In some embodiments, a plurality of knowledge terms for a respective cellular constituent module is filtered based on contextualization score, where, when the contextualization score for a respective knowledge term fails to satisfy a significance criterion, the respective knowledge term is not assigned to the respective module, and when the contextualization score for the respective knowledge term satisfies the significance criterion, the respective knowledge term and the corresponding contextualization score is included in the knowledge construct. For instance, in some embodiments, a respective plurality of knowledge terms is assigned to a respective cellular constituent module, each respective knowledge term having a corresponding weight (e.g., a p-value), and the plurality of knowledge terms is ranked in order of their corresponding weights. Knowledge terms having weights that satisfy the significance criterion are retained and used for annotation and contextualization of the respective module, while knowledge terms having weights that do not satisfy the significance criterion are removed.

In an example embodiment, cellular constituent modules (e.g., gene modules) are contextualized by transforming each vector into a list and enriching each cellular constituent module using GSEA and various public databases of gene sets and other data modalities, such as GWAS. Labeling is weighted by GSEA p-values and annotations of cellular constituent modules with knowledge terms are ranked in decreasing order of weights. Labels are filtered using a significance threshold for GSEA, such that when the GSEA produces a statistically significant result, the cellular constituent module is labeled with knowledge terms such as known pathways, biological processes, risk alleles, biomarkers, genetic variants, and/or driving transcription factors, receptors, and kinases.

In some embodiments, the contextualization comprises labeling each respective cellular constituent module in the plurality of cellular constituent modules with one or more labels obtained from a public database. In some embodiments, public databases include, but are not limited to, the NIH Gene Expression Omnibus (GEO), EBI ArrayExpress, NCBI, BLAST, EMBL-EBI, GenBank, Ensembl, the KEGG pathway database, the Library of Integrated Network-based Cellular Signatures (LINCS) L1000 dataset, the Reactome pathway database, the Gene Ontology project, and/or any disease-specific database. In some embodiments, the public database is any public knowledge database known in the art.

Referring to Block 310, the method includes pruning the activation data structure by removing from the activation data structure one or more cellular constituent modules that fail to associate with a known cellular pathway, a biological process, a transcription factor, a cell receptor, or a kinase that is implicated in the cellular process of interest thereby identifying one or more cellular constituent modules in the plurality of cellular constituent modules that is associated with the cellular process of interest and, from the one or more cellular constituent modules, the plurality of cellular constituents associated with the cellular process of interest.

In some embodiments, the pruning allows for the prioritization of cellular constituent modules that are most likely to be associated with a cellular process of interest (e.g., prioritization of disease-critical and/or process-critical cellular constituent modules). In some embodiments, the pruning allows for the prioritization of candidate targets for drug discovery.

### ADDITIONAL EMBODIMENTS

Another aspect of the present disclosure provides a computer system, comprising one or more processors and memory, the memory storing instructions for performing a method for associating a plurality of cellular constituents with a cellular process of interest. The method comprises obtaining one or more first datasets in electronic form, the one or more first datasets comprising or collectively comprising, for each respective cell in a first plurality of cells, where the first plurality of cells comprises, e.g., twenty or more cells and collectively represents a plurality of annotated cell states, for each respective cellular constituent in a plurality of cellular constituents, where the plurality of cellular constituents comprises, e.g., 50 or more cellular constituents, a corresponding abundance of the respective cellular constituent in the respective cell.

A plurality of vectors is thereby accessed or formed, each respective vector in the plurality of vectors (i) corresponding to a respective cellular constituent in the plurality of constituents and (ii) comprising a corresponding plurality of elements, each respective element in the corresponding plurality of elements having a corresponding count representing the corresponding abundance of the respective cellular constituent in a respective cell in the first plurality of cells.

The plurality of vectors is used to identify each cellular constituent module in a plurality of cellular constituent modules, each cellular constituent module in the plurality of cellular constituent modules including a subset of the plurality of cellular constituents, where the plurality of cellular constituent modules are arranged in a latent representation dimensioned by (i) the plurality of cellular constituent modules and (ii) the plurality of cellular constituents or a representation thereof (e.g., where the plurality of cellular constituent modules comprises more than ten cellular constituent modules).

The method further includes obtaining one or more second datasets in electronic form, the one or more second datasets comprising or collectively comprising, for each respective cell in a second plurality of cells, where the second plurality of cells comprises, e.g., twenty or more cells and collectively represents a plurality of covariates possibly informative of the cellular process of interest, for each respective cellular constituent in the plurality of cellular constituents, a corresponding abundance of the respective cellular constituent in the respective cell. A cellular constituent count data structure dimensioned by (i) the second plurality of cells and (ii) the plurality of cellular constituents or the representation thereof is thereby obtained.

An activation data structure is formed by combining the cellular constituent count data structure and the latent representation using the plurality of cellular constituents or the representation thereof as a common dimension, where the activation data structure comprises, for each cellular constituent module in the plurality of cellular constituent modules, for each cellular constituent in the plurality of cellular constituents, a respective activation weight.

The method further comprises training a model using a difference between (i) a prediction of an absence or presence of each covariate in the plurality of covariates in each cellular constituent module represented in the activation data structure upon input of the activation data structure into the model and (ii) actual absence or presence of each covariate in each cellular constituent module, where the training adjusts a plurality of covariate weights associated with the model responsive to the difference. The plurality of covariate weights comprises, for each respective cellular constituent module in the plurality of cellular constituent modules, for each respective covariate, a corresponding weight indicating whether the respective covariate correlates, across the activation data structure, with the respective cellular constituent module.

The plurality of covariate weights is used, upon training the model, to identify one or more cellular constituent modules in the plurality of cellular constituent modules that is associated with one or more covariates in the plurality of covariates, thereby associating a plurality of cellular constituents with the cellular process of interest.

Another aspect of the present disclosure provides a non-transitory computer-readable medium storing one or more computer programs, executable by a computer, for associating a plurality of cellular constituents with a cellular process of interest, the computer comprising one or more processors and a memory, the one or more computer programs collectively encoding computer executable instructions that perform a method.

The method comprises obtaining one or more first datasets in electronic form, the one or more first datasets comprising or collectively comprising, for each respective cell in a first plurality of cells, where the first plurality of cells comprises, e.g., twenty or more cells and collectively represents a plurality of annotated cell states, for each respective cellular constituent in a plurality of cellular constituents, where the plurality of cellular constituents comprises, e.g., 50 or more cellular constituents, a corresponding abundance of the respective cellular constituent in the respective cell.

A plurality of vectors is thereby accessed or formed, each respective vector in the plurality of vectors (i) corresponding to a respective cellular constituent in the plurality of constituents and (ii) comprising a corresponding plurality of elements, each respective element in the corresponding plurality of elements having a corresponding count representing the corresponding abundance of the respective cellular constituent in a respective cell in the first plurality of cells.

The plurality of vectors is used to identify each cellular constituent module in a plurality of cellular constituent modules, each cellular constituent module in the plurality of cellular constituent modules including a subset of the plurality of cellular constituents, where the plurality of cellular constituent modules are arranged in a latent representation dimensioned by (i) the plurality of cellular constituent modules and (ii) the plurality of cellular constituents or a representation thereof (e.g., where the plurality of cellular constituent modules comprises more than ten cellular constituent modules).

The method further includes obtaining one or more second datasets in electronic form, the one or more second datasets comprising or collectively comprising, for each respective cell in a second plurality of cells, where the second plurality of cells comprises, e.g., twenty or more cells and collectively represents a plurality of covariates possibly informative of the cellular process of interest, for each respective cellular constituent in the plurality of cellular constituents, a corresponding abundance of the respective cellular constituent in the respective cell. A cellular constituent count data structure dimensioned by (i) the second plurality of cells and (ii) the plurality of cellular constituents or the representation thereof is thereby obtained.

An activation data structure is formed by combining the cellular constituent count data structure and the latent representation using the plurality of cellular constituents or the representation thereof as a common dimension, where the activation data structure comprises, for each cellular constituent module in the plurality of cellular constituent modules, for each cellular constituent in the plurality of cellular constituents, a respective activation weight.

The method further comprises training a model using a difference between (i) a prediction of an absence or presence of each covariate in the plurality of covariates in each cellular constituent module represented in the activation data structure upon input of the activation data structure into the model and (ii) actual absence or presence of each covariate in each cellular constituent module, where the training adjusts a plurality of covariate weights associated with the model responsive to the difference. The plurality of covariate weights comprises, for each respective cellular constituent module in the plurality of cellular constituent modules, for each respective covariate, a corresponding weight indicating whether the respective covariate correlates, across the activation data structure, with the respective cellular constituent module.

The plurality of covariate weights is used, upon training the model, to identify one or more cellular constituent modules in the plurality of cellular constituent modules that is associated with one or more covariates in the plurality of covariates, thereby associating a plurality of cellular constituents with the cellular process of interest.

Another aspect of the present disclosure provides a computer system having one or more processors, and memory storing one or more programs for execution by the one or more processors, the one or more programs comprising instructions for performing any of the methods and/or embodiments disclosed herein. In some embodiments, any of the presently disclosed methods and/or embodiments are performed at a computer system having one or more processors, and memory storing one or more programs for execution by the one or more processors.

Another aspect of the present disclosure provides a non-transitory computer readable storage medium storing one or more programs configured for execution by a computer, the one or more programs comprising instructions for carrying out any of the methods disclosed herein.

### III. EXAMPLES

The Examples below provide performance measures and practical applications of models that identify activation values for cellular constituent modules.

### Example 1. Predicting Shared and Orthogonal Cell Type and Treatment Modules for Megakaryocyte Differentiation

In this example, a first dataset is obtained in electronic form that comprises, for each respective cell in a first plurality of cells, where the first plurality of cells comprises twenty or more cells and collectively represents a plurality of annotated cell states, for each respective cellular constituent in a plurality of cellular constituents, where the plurality of cellular constituents comprises 50 or more cellular constituents, a corresponding abundance of the respective cellular constituent in the respective cell, thereby accessing or forming a plurality of vectors, each respective vector in the plurality of vectors (i) corresponding to a respective cellular constituent in the plurality of constituents and (ii) comprising a corresponding plurality of elements, each respective element in the corresponding plurality of elements having a corresponding count representing the corresponding abundance of the respective cellular constituent in a respective cell in the first plurality of cells.

In particular, in this example, scRNA-seq is collected from cells of an in vitro model of differentiating CD34 stem cells, perturbed with Megakaryocyte stimulating media (MkP) and different interventions (and thus representing different annotated states). This scRNA-seq data was pre-processed through filtering and normalization steps. This resulted in a pre-processed count matrix 110 of the form illustrated in the upper portion of FIG. 4 and contained between 100 to 8000 cellular constituents with a high signal-to-noise ratio.

Further in this example, the plurality of vectors (e.g., the counter matrix 110 of the upper portion of FIG. 4) is used to identify each cellular constituent module in a plurality of cellular constituent modules. Each cellular constituent module in the plurality of cellular constituent modules includes a subset of the plurality of cellular constituents. The plurality of cellular constituent modules were arranged in a latent representation (e.g., latent representation 118 in the lower portion of FIG. 4) dimensioned by (i) the plurality of cellular constituent modules and (ii) the plurality of cellular constituents or a representation thereof. To compute the cellular constituents modules, a correlation-based or sparse auto-encoder cost function was optimized. Optimizing the correlation-based cost function amounted to computing a nearest-neighbor graph defining neighborhood relations among cellular constituents, representing each cellular constituents by a vector formed by storing the expression values for the cellular constituents in each cell (e.g., count matrix 110 of upper portion of FIG. 4), and computing correlations among cellular constituents. Cellular constituents whose abundance values had a high correlation (e.g., more than 0.5, more than 0.6, more than 0.7) across the first plurality of cells end up being nearest neighbors, and form a cellular constituent module. In some embodiment such correlation determination and clustering was performed using a graph based method such as Leiden or any other graph clustering method.

The identification of the cellular constituent modules can be considered a latent representation, such as the latent representation 118 illustrated in the lower portion of FIG. 4. The latent representation includes between 50 to 500 latent dimensions, each of which is numbered and represents a single cellular constituent module (latent representation 118 Y-axis), containing between 2 and 300 cellular constituents (latent representation 118 X-axis). The weights of the latent representation 118 can be represented by an indicator matrix, with an entry 1/n_genes if a cellular constituent is part of a cellular constituent module, and an entry 0 if it is not. Here, n_genes is the number of cellular constituents in a cellular constituent module. Thus, referring to the latent representation 118 of FIG. 4, weights 122 for a particular cellular constituent have a value of “0” in individual cellular constituent modules that the particular cellular constituent is not present in and have a value of “1” in individual cellular constituent modules that the particular cellular constituent is present in. It will be appreciated that any labeling scheme that indicates which cellular constituents are in which cellular constituent modules in the latent representation 118 can be used.

In this example, a second dataset was received in electronic form that contained, for each respective cell in a second plurality of cells, where the second plurality of cells comprises twenty or more cells and collectively represents a plurality of covariates possibly informative of the cellular process of interest, for each respective cellular constituent in the plurality of cellular constituents, a corresponding abundance of the respective cellular constituent in the respective cell, thereby obtaining a cellular constituent count data structure dimensioned by (i) the second plurality of cells and (ii) the plurality of cellular constituents or the representation thereof. In this example, the second dataset is an appropriately normalized scRNA-seq count matrix of shape n_cells×n_genes that included cells for each of the covariates of interest. This second dataset has the form of count data structure (covariate set) 160 of FIG. 5.

Next in this example, an activation data structure 170 (e.g., of the form on the lower right hand side of FIG. 5) was formed by combining the above described cellular constituent count data structure (of the form of count data structure 160 on the lower left hand side of FIG. 5) and the latent representation (e.g., of the form of latent representation 118 of the upper portion of FIG. 5) using the plurality of cellular constituents or the representation thereof as a common dimension. As illustrated in FIG. 5, the activation data structure 170 comprises, for each cellular constituent module in the plurality of cellular constituent modules, for each cell in the second plurality of cells, a respective activation weight. In other words, the matrix multiplication of the latent representation 118 by the second dataset (counter data structure 160) maps the scRNA-seq data into a space, illustrated by activation data structure 170 in the lower right hand side of FIG. 5, in which each cell (Cell 1, . . . , Cell G) is characterized by its cellular constituent module activations (Act₁₋₁, . . . , Act_{K-G}) in which the resulting matrix representation has dimensions n_cells×n_modules.

The example continues by training a model 601. For instance, consider the case where model 601 includes the weights of row 604-1 (Weight₁₋₁ through Weight_{1-W}) to represent the extent to which covariates 1 through W respectively activate cellular constituent module 1. This model 601 is trained on the elements of row 640 of activation data structure 170, which provide the extent to which each of the covariates 1, . . . , G activate cellular constituent module 1. In this training, first a representation of the covariate associated with cell 1 is inputted into the model 601. Responsive to this input, the model 601 for cellular constituent module 1 outputs an activation value, termed Pred. Value₁ in the nomenclature of FIG. 6. This output activation value is compared to the actual activation value for the covariate associated with cell 1, which is Act₁₋₁ of activation data structure 170. Next, a representation of the covariate associated with cell 2 is inputted into the model 601. In response to this input, the model outputs an activation value (Pred. Value₂). This output activation value is compared to the actual activation value for the covariate associated with cell 2 of row 640, which is Act₁₋₂ of activation data structure 170. This process proceeds through cell G. A representation of the covariate associated with cell G is inputted into the model 601. In response to this, the model will output an activation value (Pred. Value_{G}). This output activation value is compared to the actual activation value for cell G, which is Act_{1-G} of row 640 of activation data structure 170. In this example, W and G have the same value. In this way, there is a resulting prediction (Pred. Value) for each covariate in the plurality of covariates used to derive the activation data structure as outlined in FIG. 5 for cellular constituent module 1. The above-described calculated predictions (of the activation value) are compared to the above-described actual activation values for each of these covariates and the differences between the predicted and actual activation values are used to further train the model 601 using back-propagation and related model refinement techniques. In this example, the model, illustrated as element 601 in FIG. 6, is a one-layer neural net model (SoftMax/Logistic Regression) for each cellular constituent module. In this optimization, the input features for each model 601 are the representations of the covariates associated with each column of the activation data structure 170.

Referring to FIG. 6, the parameters of the classification model 601 are weights 1-1, . . . 1-W, . . . , K-1, . . . , K-W, for the cellular constituents modules, values for the weights 1-1, . . . 1-W, . . . , K-1, . . . , K-W are determined using categorical cross-entropy loss in a multi-task formulation in this example. Classifying each categorical (batch, donor, disease status, etc.) corresponds to one such cost function each. Each of these cost functions receive the same weighting factor. In this example the covariates were cell type labels, flow cytometry readouts, and coupling of a collection of public knowledge bases.

The example continued by identifying, using the plurality of covariate weights upon training the model 601, one or more cellular constituent modules in the plurality of cellular constituent modules that was associated with one or more covariates in the plurality of covariates, thereby associating a plurality of cellular constituents with the cellular process of interest. Here, the trained model 601 offers trained weights 1-1, . . . 1-W, . . . , K-1, . . . , K-W for each of the covariates, and hence revealed cellular constituent modules that inform for megakaryocyte differentiation. For instance, from the analysis of these trained weights, the top 10 most strongly activated cellular constituent modules were identified. Table 2 provides the identities of the top 10 most strongly activated cellular constituent modules, along with associated annotations (KEGG: Kyoto Encyclopedia of Genes and Genomes; REAC: Reactome; TF: transcription factors; GO: gene ontology; BP: biological process; MF: molecular function). Among these, as presented in Table 2, four cellular constituent modules were found to be significantly associated with platelet activation, the cellular processes known to associate with the phenotypic endpoint of thrombocytopenia. Comparison of shared and orthogonally activated cellular constituent modules after treatment with MkP and additional interventions (Compound N and Compound P) is illustrated in FIG. 7. For example, cellular constituent module 77 was identified as having shared activation after both Compound P and MkP treatment.

TABLE 2
Cellular constituent modules activated by interventions.

| | | | | | |
|---|---|---|---|---|---|
| Module | KEGG | REAC | TF | GO:BP | GO:MF |

Modules activated in MkP condition

| | | | | | |
|---|---|---|---|---|---|
| 4 | Platelet | Platelet | | Platelet | Molecular |
| | activation | activation, | | activation | function |
| | | signaling and | | | regulator |
| | | aggregation | | | |
| 1 | Platelet | Hemostasis | | Response to | |
| | activation | | | wounding | |
| 0 | Platelet | Platelet | | Cell activation | |
| | activation | degranulation | | | |
| 6 | | | | Antibiotic | Hemoglobin |
| | | | | catabolic | binding |
| | | | | process | |
| 3 | Steroid | Platelet | Factor: Sp1; | Secondary | Protein |
| | biosynthesis | activation, | motif: NNGGGGCGGGGNN; | alcohol | binding |
| | | signaling and | match class: 0 | biosynthetic | |
| | | aggregation | | process | |
| 80 | Allograft | Endosomal/ | | Antigen | |
| | rejection | vacuolar | | processing and | |
| | | pathway | | presentation of | |
| | | | | exogeno . . . | |
| 7 | Cell cycle | Cell cycle | Factor: YB-1; | Mitotic cell | Enzyme |
| | | | motif: NNNNCCAATNN; | cycle process | binding |
| | | | match class: 1 | | |
| 8 | Ribosome | Selenoamino | | Nuclear- | Structural |
| | | acid metabolism | | transcribed | constituent of |
| | | | | mRNA catabolic | ribosome |
| | | | | process | |
| 2 | Cell cycle | S phase | Factor: E2F-2; | Cellular | Nucleic acid |
| | | | motif: GCGCGCGCGYW; | metabolic | binding |
| | | | match class: 1 | process | |

Module uniquely activated in Compound P-treated condition

| | | | | |
|---|---|---|---|---|
| 77 | JAK-STAT | Interleukin-4 | Response to | 1- |
| | signaling | and Interleukin- | cytokine | phosphatidylinositol- |
| | pathway | 13 signaling | | 3-kinase regulator |
| | | | | activation |

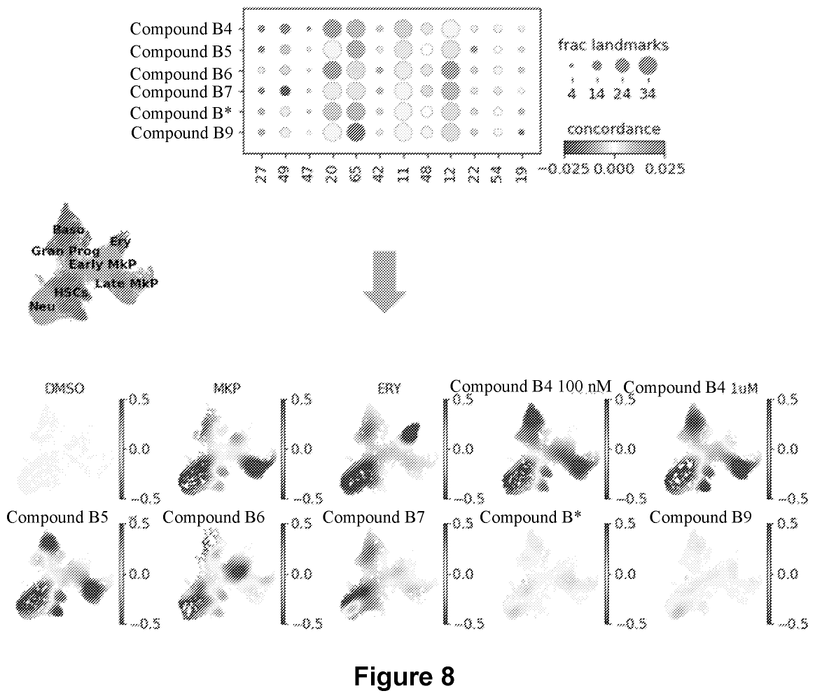

The approach was validated using the in vitro model of megakaryocyte differentiation from CD34 stem cells. Module activation and differentiation from CD34 stem cells in response six 6 different compounds (Compound B4, Compound B5, Compound B6, Compound B7, Compound B*, and Compound B9) are indicated in FIG. 8. The top panel shows activation of modules (labeled as numbers along the x-axis) in response to each of the six compound treatments. In the top panel of FIG. 8, module size (e.g., number of elements) is indicated by the size of the circle. Cellular constituent modules with negative concordance are indicated by asterisks, whereas all other cellular constituent modules not so marked have positive concordance. Degree of concordance is indicated by the intensity of shading in accordance with the legend (“concordance”).

Differentiated lineages are indicated in the bottom panel of FIG. 8. The dot plot shows gene module activation of scRNA-seq gathered across the six compound treatment conditions (Baso: basophils; MkP: megakaryocytes), where the degree of activation is indicated by the intensity of shading. Regions of the dot plot indicating positive activation are circled. In particular, module 20 was shared between megakaryocytes and basophils. The compounds that target module 20 (e.g., Compound B4, Compound B5, and Compound B9, as shown in the top panel) induce both of these cell types, whereas the compounds that do not activate module 20 do not give rise to both lineages.

### Example 2. Correcting Epithelial Inflammation in a Patient-Derived Pre-Clinical Organoid Model of COPD

In COPD and other muco-obstructive pulmonary diseases, goblet cell hyperplasia and resultant mucus hypersecretion contributes to patient morbidity and mortality. Inflammatory signaling such as IL-13 induces basal cells to predominantly differentiate to disease-driving goblet cells, at the expense of ciliated cells and club cells. To identify and target the disease-driving cell behaviors underlying these alterations in epithelial lineage differentiation, scRNA-seq data was generated using an air liquid interface (ALI) model of IL-13 induced goblet cell hyperplasia.

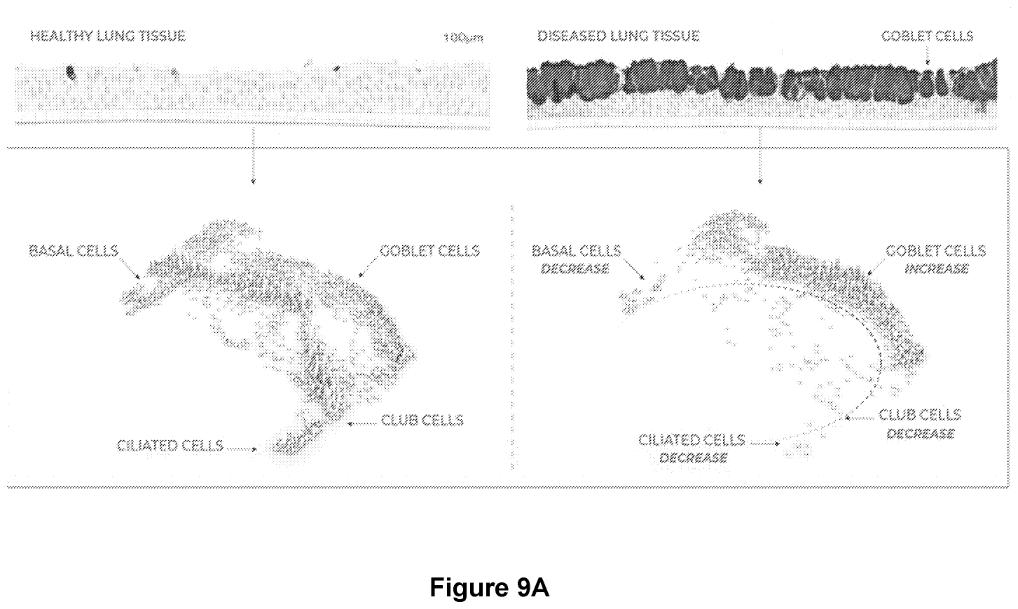

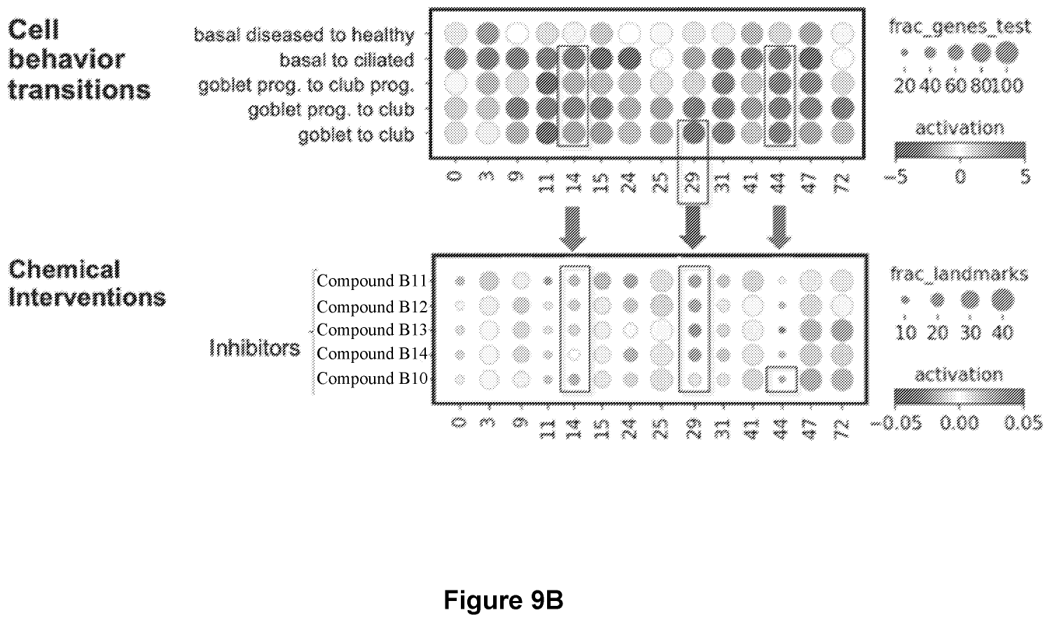

FIG. 9A illustrates that histology and scRNA-seq manifold of the air liquid interface (ALI) model of IL-13 induced goblet cell hyperplasia. IL-13 alters lineage differentiation and induces multiple cell behavior shifts in lung epithelium along with the observed goblet cell hyperplasia, which results in establishment of a detrimental mucus layer. This indicated that multiple disease cell behaviors may contribute to the disease phenotype in a complex system like the airway epithelium.

FIG. 9B illustrates cellular constituent module activation in different disease-modulating cell state transitions (e.g., cell behavior transitions). Module size (e.g., number of cellular constituents) is indicated by the size of the circle, and negative activation is indicated by asterisks. Degree of activation is indicated by the intensity of shading in accordance with the legend (“activation”). Highlighted modules indicate molecular changes specific to a cell state (thin arrow) or across multiple cell states (thick arrows). Predictions that a BRD4 inhibitor Compound B10 targets modules associated with multiple cell states was further validated by studies using chemical interventions.

Thus, by mapping cellular constituent modules to disease-modulating cell state transitions, it was possible to determine that some transcriptional changes are state-specific, while some are associated with multiple cell states. In particular, Compound B10 is predicted to modulate multiple cellular transcriptional programs of interest, which was validated from observations of the scRNA-seq data gathered from the ALI model system.

### Example 3. Predicting Translation of T-Cell Exhaustion Reversal Through Tool Compounds, Impact on Patient Response to Checkpoint Inhibitors, and Survival

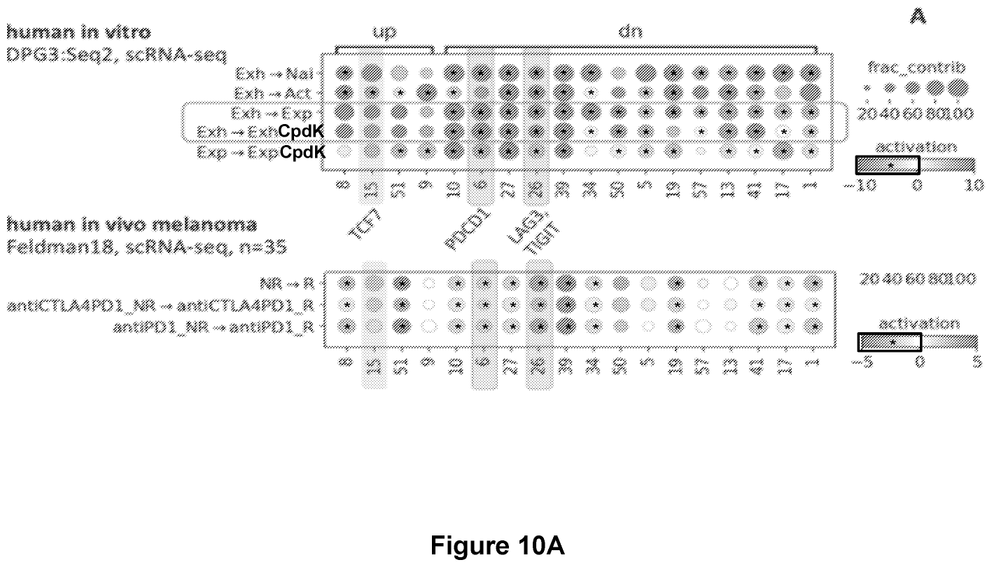

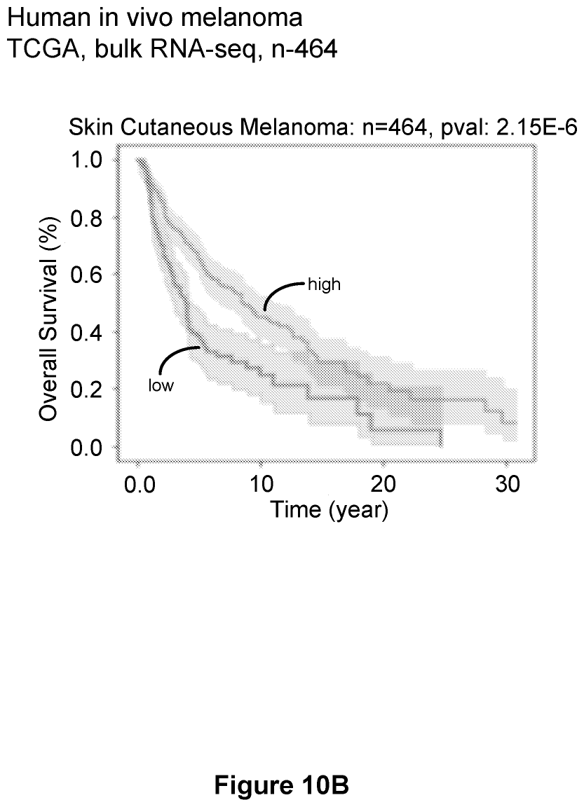

By using a cellular constituent modules trained on scRNA-seq of an in vitro T cell exhaustion model system and mapping it on public patient data, it was determined that some of the in vitro biology finds correspondence in the patient data, where some does not. For instance, comparison of the top panel (in vitro model data) with the bottom panel of FIG. 10A (human in vivo data) reveals differences in the proportions and/or degrees of positive or negative activations for various cellular constituent modules. Negative activation is indicated by asterisks. Degree of activation is indicated by the intensity of shading in accordance with the legend (“activation”).

However, all cellular constituent modules that are related to key marker cellular constituent (e.g., modules 15, 6, and 26, highlighted by shaded boxes) display the same changes in vitro upon treatment with the tool compound K as observed when comparing responders to non-responders in the in vivo data.

As illustrated in FIG. 10B, mapping module 15 onto the survival data of the Cancer Genome Atlas (TCGA) shows a significant association with survival, similar to previous observations in breast cancer (not shown). Thus, predictions obtained from the methods of the present disclosure can be used to carve out the cellular constituents of identified cellular constituent modules as initial targets for learning from a screening iteration.

### Example 4. Informing Disease Modules Using Statistical Genetics to Predict Modules that Enrich for Known Disease Traits

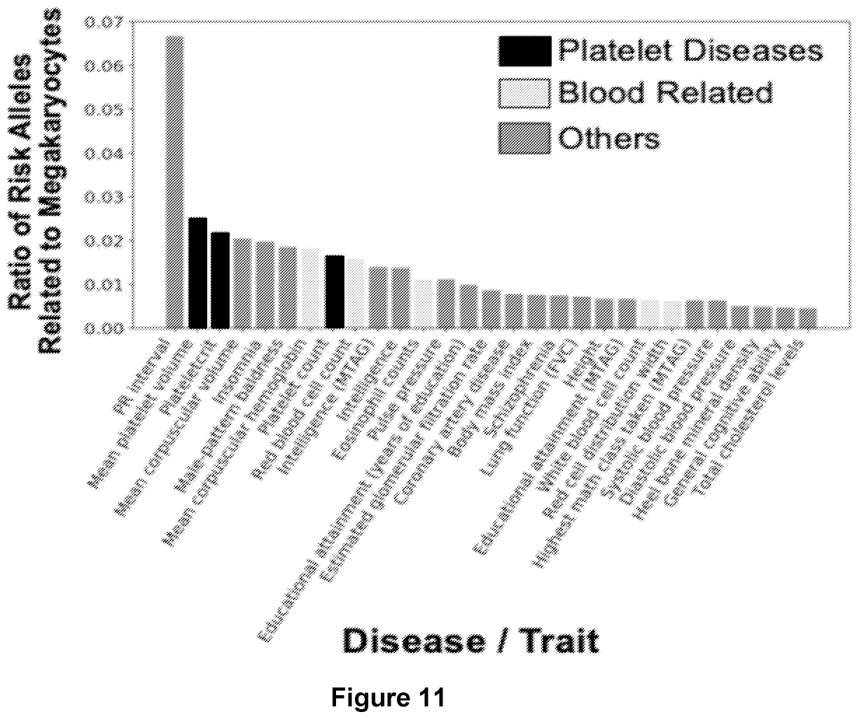

Many diseases have unknown etiology, where disease-causing cellular programs are not fully known. Genome-wide association studies (GWAS) identify single nucleotide polymorphisms (SNPs) that are enriched in individuals suffering from a given disease. The modeling architecture allows the input of GWAS data to enrich cell states and modules. To test the predictive capability of the approach, transcription factors from the megakaryocyte differentiation system described above were input into a model in accordance with the present disclosure. As illustrated in FIG. 11, predicting the disease associations for the signature predicts diseases linked to platelets (black bars), blood-related disease (light grey bars), and others (medium grey bars), among 3616 total traits assessed. This approach also makes unexpected predictions, including that megakaryocyte transcriptional signatures are associated with PR intervals, commonly associated with atrial and atrioventricular electrical activity.

### Example 5. Predicting Malignant Cells by Integrating Copy Number Variations for scRNA-Seq of Cells Derived from Dissociated Breast Cancer Patient Samples

Somatic copy number variations (CNVs) are ubiquitous in cancer cells. Arising from genomic instability, CNVs are typically identified from DNA sequencing data and are detected as amplifications or deletions of large portions of genomes. Understanding CNVs in tumors can provide insight into how genetic changes correlate with or drive phenotypes.

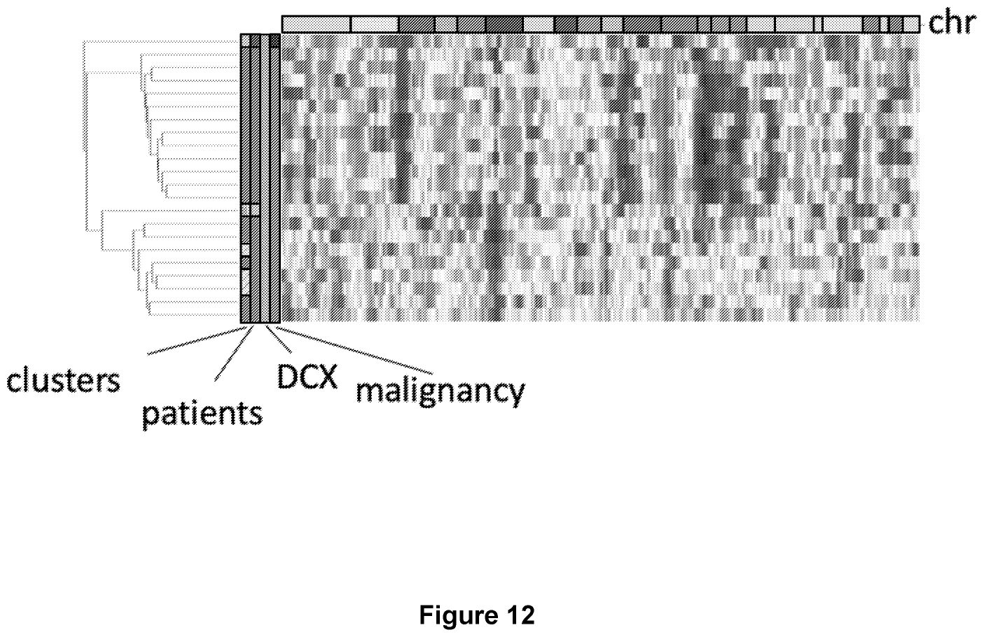

CNV information was proxied by scoring a cell's gene expression density on chromosomes. The approach has been shown to correlate well with whole-genome-sequencing based CNV characterization. Inputting the CNV profile for each gene allows the formation of connections between cell states and modules and their genetic architecture. For example, as illustrated in the heatmap in FIG. 12, cell malignancy can be predicted through its CNV profile.

Using publicly available data, DCX+ cells in triple negative breast cancer scRNA-seq data were studied and investigated to determine whether these cells were healthy neural progenitors or malignant cancer cells. FIG. 12 plots the CNV profile through chromosomal deletions and duplications (e.g., degree indicated by intensity of shading) for genes segregated by chromosome (x-axis). CNV profiles were clustered and each cell (row) was annotated by clusters inferring malignancy and DCX+ state (e.g., top cluster characterized by greater degree of copy number variations; bottom cluster characterized by lesser degree of copy number variations). As confirmation of the validity of the approach, immune cells such as T cells and macrophages were found to be not malignant and exhibited low levels of CNVs.

### Example 6. Predicting Interactions of Immune Cells and Malignant Cells in Breast Cancer

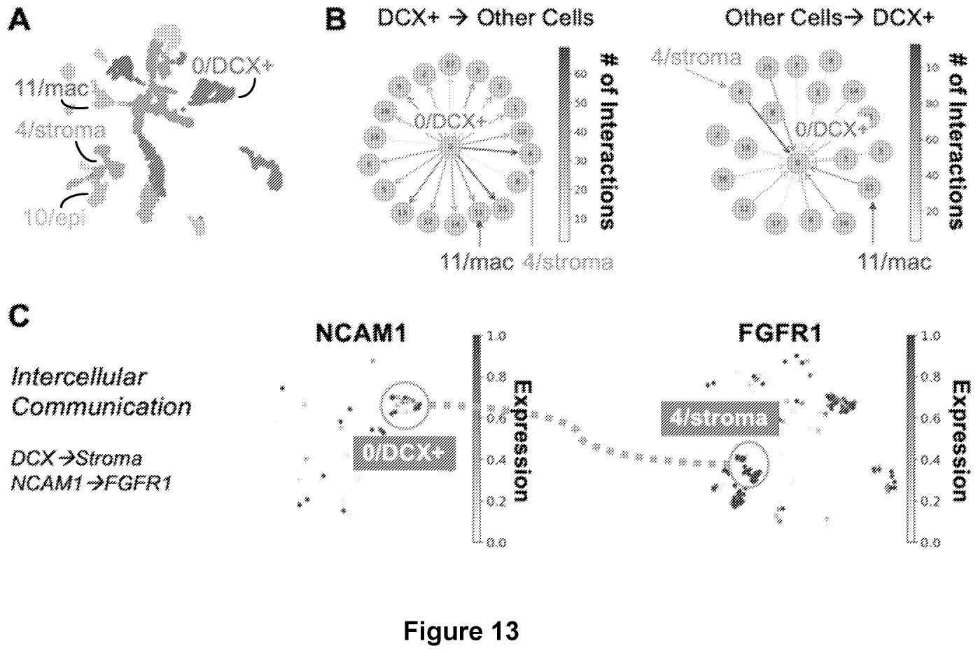

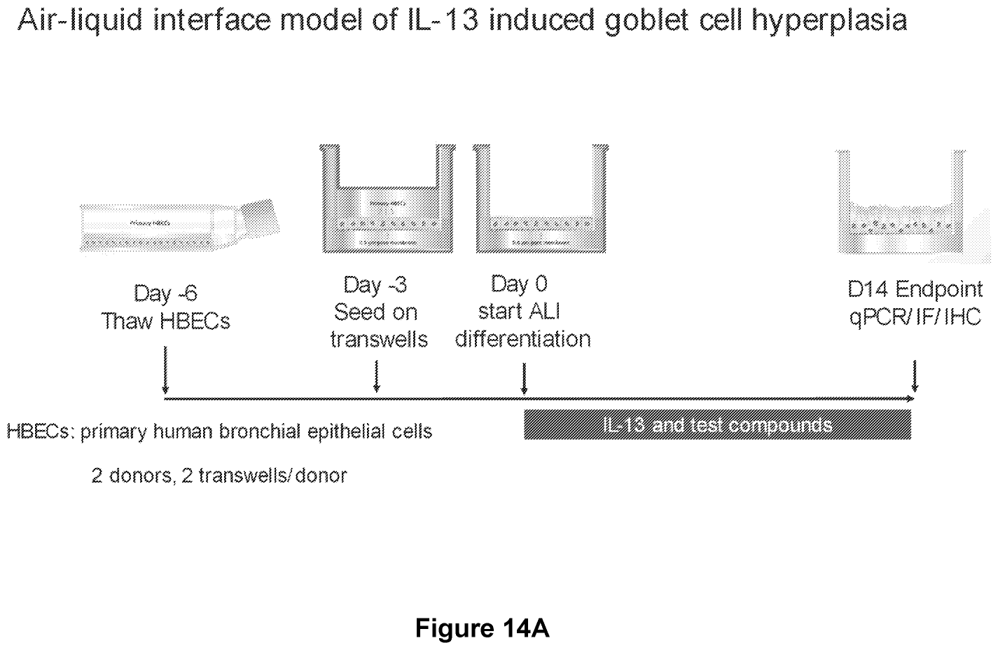

The roles of DCX+ neural progenitor cells in the tumor microenvironment were further examined by identifying DCX+ cells in triple negative breast cancer (TNBC) single cell data and determining that they were associated with worse outcomes in adenocarcinomas and breast cancer patients. Using copy number variation inference, it was predicted that these DCX+ cells are actually malignant. FIG. 13 shows the results of an investigation into how these cells interact with other cells in the tumor microenvironment. By scoring expression of ligand receptor pairs across cell types, it was observed that stromal cells and macrophages display the highest number of significant interactions with DCX+ cells.

With reference to FIG. 13, A: a Uniform Manifold Approximation and Projection (UMAP) plot of different cell types in a tumor environment (e.g., DCX+, macrophages, stroma, and epithelial) is illustrated. Numbers indicate cellular constituent modules expressed by DCX+ cells and other cell types (e.g., 0/DCX+; 11/macrophages; 4/stroma; and 10/epithelial). B: the number of interactions between DCX+ cells and other cell types are indicated by circled numbers as well as by arrow shading intensity (darker shading indicates a greater number of interactions). Circles denote cellular constituent modules expressed by DCX+ and other cell types. In the left panel, DCX+ cells are source cells, while in the right panel, DCX+ cells are target cells. C: Investigation of specific molecular interactions, revealed that NCAM1 (a neural cell adhesion molecule), a neural progenitor cell marker in DCX+ cells, communicated predominantly with 3 other cell types including itself, stroma, and basal epithelial cells by co-binding FGF2 with FGFR1 receptor. Expression of NCAM1 in DCX+ cells and expression of FGFR1 in stroma cells is indicated by intensity of shading across the UMAP plot, in accordance with the legend (“Expression”). Academic literature suggests that there exists an NCAM1-FGF2-FGFR1 signaling axis that regulates tumor survival and migration. Thus, the findings obtained using cellular constituent modules (and/or models for identifying the same) can be used to prioritize critical cell targets for downstream application.

### Example 7. Air-Liquid Interface (ALI) Differentiation of Primary Human Bronchial Epithelial Cells (HBECs) is an Accepted In Vitro Model of IL-13—Induced Goblet Cell Hyperplasia

Expansion of HBEC culture: Primary HBECs (Lonza) were thawed into T75 flasks in PneumaCult Ex Plus medium (STEMCELL Technologies) and cultured for 2-3 days until 80% confluency. Cells were then dissociated for 10 min using Animal Component-Free (ACF) Cell Dissociation Kit (STEMCELL Technologies) and plated onto the apical chamber of 6.5 mm wide, 0.4 μm pore-sized Transwells (STEMCELL Technologies) at 3.3e4 cells per well with PneumaCult-Ex medium on both sides of the membrane.

ALI culture: After the cells reached 50-80% confluency on transwells (1-2 days later), cultures were transferred to ALI conditions. Medium was removed from the apical and basal chambers and replaced only in the basal chamber with complete PneumaCult-ALI Medium (STEMCELL Technologies). Where appropriate, 0.3-1 ng/ml IL-13 (R&D) and test compounds were added to the basal chamber. Medium containing IL-13 and test compounds was replenished every 2-3 days, and excess mucus was washed from the apical chamber with 200 μl warm PBS−/− twice a week starting on day 7.

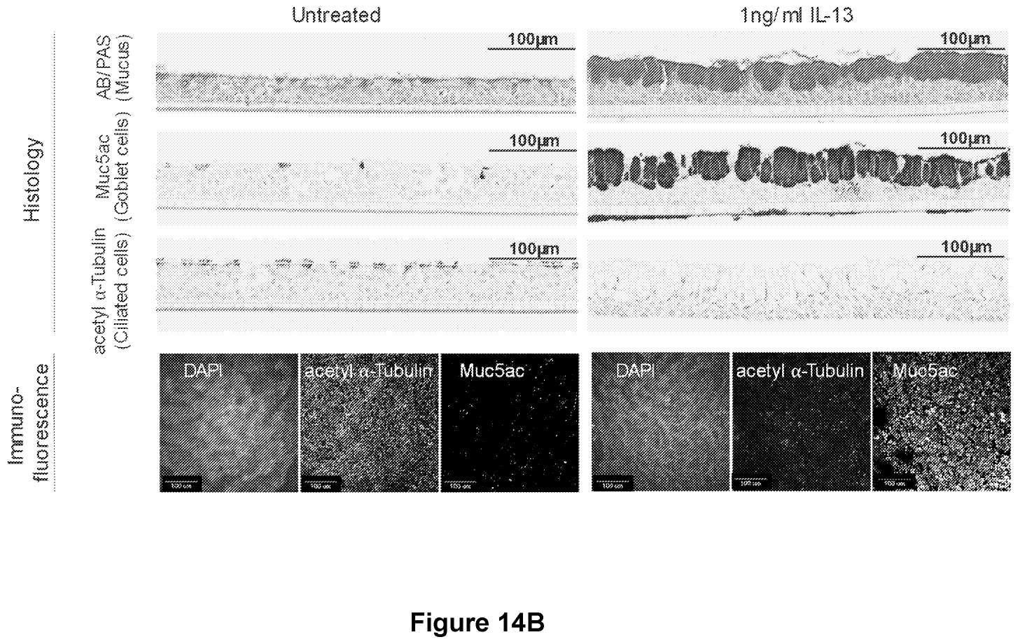

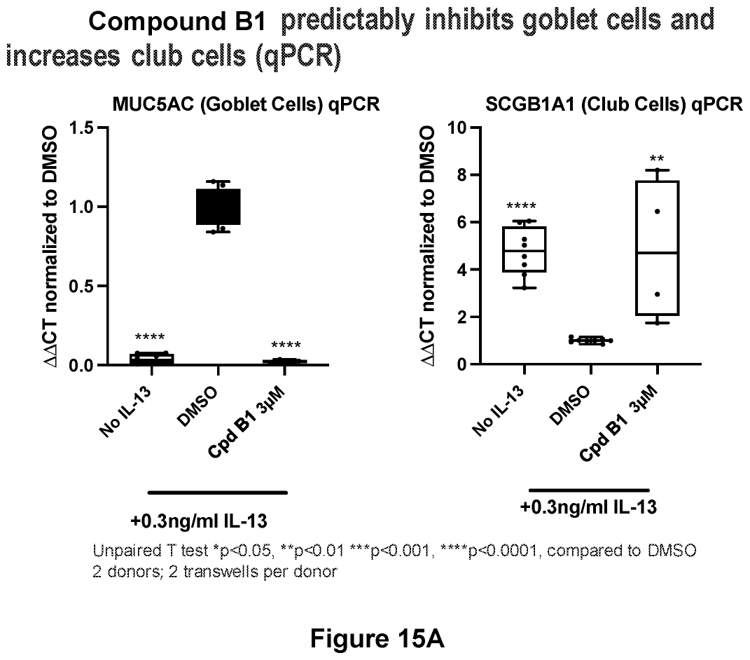

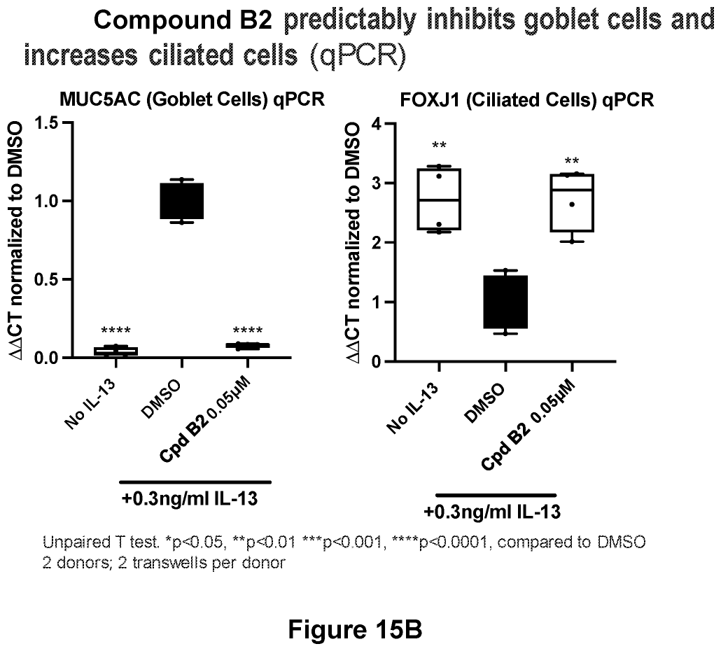

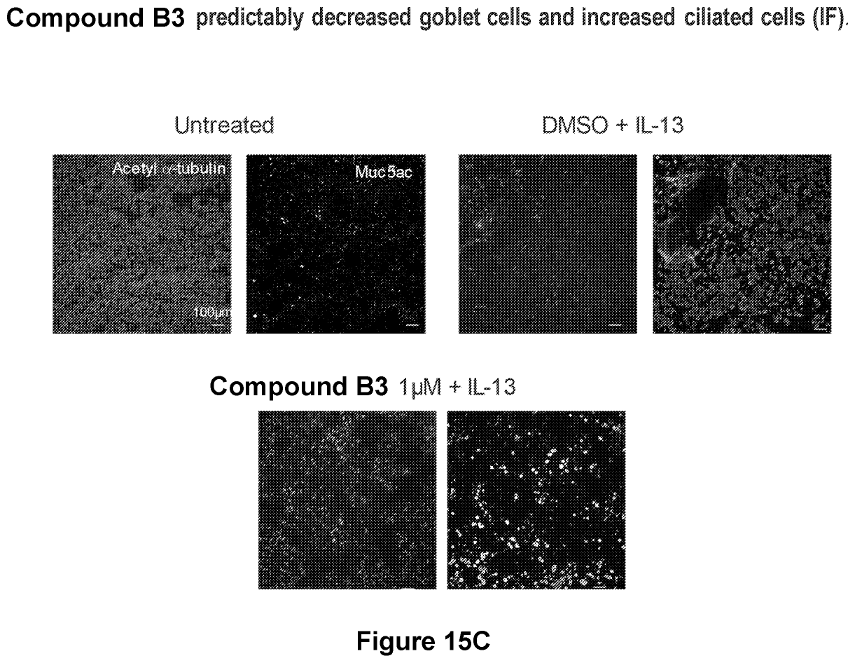

Pseudostratified airway epithelium was formed by day 14 of ALI differentiation. Goblet (Muc5ac+, AB/PAS+) and ciliated cells (Acetyl α-tubulin+) were visualized via immunofluorescence or histology. Immunofluorescence allows for a top down view of the most apical layer of cells, while histology presents a cross-section of the pseudostratified epithelium and is amenable for image quantification. In addition, qPCR of ciliated (Foxj1) and goblet (Muc5ac) marker expression was used as a sensitive and quantitative readout of epithelial cell type distribution. FIGS. 14A-B and 15A-C illustrate that ciliated cells arise only in the untreated condition, while, conversely, goblet cells arise only in the IL-13-treated condition, modeling goblet cell hyperplasia.

Immunofluorescence readout: For the immunofluorescence (IF) readout, transwells were fixed with 4% formaldehyde (Sigma) for 30 min and washed 3× in PBS−/−. Transwell membranes were excised with a scalpel and blocked/permeabilized for 1 hour in solution containing 2.5% goat serum (Invitrogen), 2.5% donkey serum (Sigma), 1% BSA (ThermoFisher), and 0.2% Triton X-100 (MP Biomedicals) in PBS−/−. Membranes were then incubated with primary antibodies (Muc5ac, clone 45M1, ThermoFisher and acetyl α-tubulin, clone 6-11B-1, Sigma) overnight at 4° C., washed 3× in PBS−/−, incubated with secondary antibodies (goat anti-mouse IgG1 Alexa 555 and goat anti-mouse IgG2b Alexa 647, ThermoFisher) for 45 min at room temperature, washed again, and mounted apical side down onto 48 well plates with SlowFade Diamond Antifade Mountant with DAPI (Invitrogen) and covered with 8 mm coverslips (Thomas Scientific). Plates were imaged on ImageXpress Micro 4 (Molecular Devices).

Histology readout: For the histology readout, transwells were fixed with 4% formaldehyde (Sigma) for 4 hours, washed 3× in PBS−/−, and shipped to HistoTox Labs (HTL), Boulder Colo., in PBS-filled 50 ml tubes. At HTL, samples were trimmed, placed in individually labeled cassettes, and processed per HTL SOPs for paraffin embedding. Tissue blocks were sectioned at 4 μm onto labeled slides and stained with Hematoxylin & Eosin (H&E) and Alcian Blue/Periodic Acid Schiff (AB/PAS) dyes per HTL SOPs. Immunohistochemical (IHC) staining of Formalin-Fixed Paraffin-Embedded (FFPE) was conducted on a Leica Bond Rxm using standard chromogenic methods. For antigen retrieval (HIER), slides were heated in a pH 6 Citrate based buffer for 2 hours at 70° C. (Acetyl α-Tubulin, clone EPR16772 Abcam), or pH 9 EDTA based buffer for 2 hours at 70° C. (Muc5ac, clone 45M1 ThermoFisher), followed by a 30 minute antibody incubation (acetyl α-tubulin) or 45 minute antibody incubation (Muc5ac). Antibody binding was detected using an HRP-conjugated secondary polymer, followed by chromogenic visualization with diaminobenzidine (DAB). A Hematoxylin counterstain was used to visualize nuclei. Slides were scanned and visualized on PathCore. Quantification of AB/PAS, acetyl α-tubulin, and Muc5ac staining signal was performed on MetaXpress (Molecular Devices) software as positive area.

qPCR readout: At day 14, transwell membranes were excised and cut in half with scalpel (VWR, 10148-884) and placed into 1.5 mL tube with 3504, of Buffer RLT (QIAGEN, 74106) and 2-mercaptoethanol (Gibco, 21985023) added at 1:100. Lysate (350 μl) was then transferred into QIAShredder Column (QIAGEN, 79656), and centrifuged at max speed for 2 minutes at +4° C. RNA extraction was performed according to the manufacturer's instructions for RNeasy Mini Kit (QIAGEN, 74106). RNA was quantified on NanoDrop One (ThermoFisher), and cDNA generated using High-Capacity cDNA Reverse Transcription Kit (Applied Biosystems, 4368814), on the Biorad C1000 Touch Thermal Cycler.

For qPCR, target genes MUC5AC-FAM-MGB (ThermoFisher, 4331182, Hs00873651_mH), human FOXJ1 FAM-MGB (ThermoFisher, Hs00230964_m1), or human SCGB1A1 FAM-MGB (ThermoFisher, 4331182|Hs00171092_m1) were duplexed with reference gene primer YWHAZ-VIC-MGB (ThermoFisher, 4448484, Hs03044281_g1) for simultaneous detection in a single qPCR reaction. Each reaction was run in triplicate, using TaqMan Fast Advanced Master Mix (ThermoFisher, 444456) according to manufacturer's protocols in Hard-Shell 384-well thin-wall PCR plates (BioRad HSP3805) on BioRad CFX 384 Touch Real-Time PCR Detection System. For qPCR analysis, technical replicates were normalized by housekeeping genes and averaged, and ΔCt values were calculated.

### Example 8. Simulated Versus Measured Impact of Chaetocin on Blood Cells (In Vitro CD34+)

Methods to generalize and prioritize, from transcriptional profiles of perturbed cell lines, compounds that will modify a cell behavior of interest were developed. See United States Patent Publication Number 2020-0020419 entitled “Methods of Analyzing Cells,” which is hereby incorporated by reference. The methods score a target cell behavior and predict a set of compounds (where each compound is considered a perturbation) that are most likely to induce that cell behavior. However, the effect of predicted compounds on overall cell type composition, apart from target cell behavior, remained unclear. To address this, the disclosed methods were developed, which allows, among other things, for the prediction of global change in cell type distribution.

Previously, the effect of 75 unique small molecule interventions in CD34+ cells was determined using single cell RNA sequencing, which allowed for assignment of CD34+ cells to one of the 10 biologically relevant clusters and measurement of the proportion of each cluster in each sample. The shift in distribution of cell types in perturbed CD34+ cells relative to the control, unperturbed cells was measured. Using this observed shift, a model that can predict the expected shift in cell type abundance in samples perturbed with unseen small molecules was trained. This model uses the score described in United States Patent Publication Number 2020-0020419 entitled “Methods of Analyzing Cells,” to quantify cell behavior transition from HSPC cells to one of the 9 target cell types (granulocyte monocyte progenitor cell, MEMP (MK-erythroid-mas), erythroid, megakaryocyte progenitor cell, megakaryocyte cell, mast cell, monocyte cell, B cell, and precursor B cell) to predict change in abundance of the target cell type under a given small molecule perturbation.

The model was evaluated using k-fold cross validation strategy and in how many cases a correct prediction was made in the direction of the change (increase or decrease in abundance) for each of the nine terminal target cell types. The direction of change for B cells was correctly predicted by the model in 66% of the cases. The direction of change for monocytes was correctly predicted by the model in 74% of the cases. The direction of change for megakaryocytes was correctly predicted by the model in 63% of the cases. The model shows positive predictive power on six (6) blood cell types, with 70% balanced accuracy on erythroid and megakaryocyte progenitor cells upon intervention with 61 compounds. As such the model shows potential value for predicting compounds that, upon intervention, elicit desired behaviors in multiple cell types, and thus for predicting compounds for treatment of complex diseases. The model further shows potential value for optimizing such compound “hits” into “leads” (e.g., new chemical entities) against behaviors in multiple cell types, and for minimizing and/or avoiding potential unwanted side effects or toxicities associated with inadvertently modulating healthy cells.

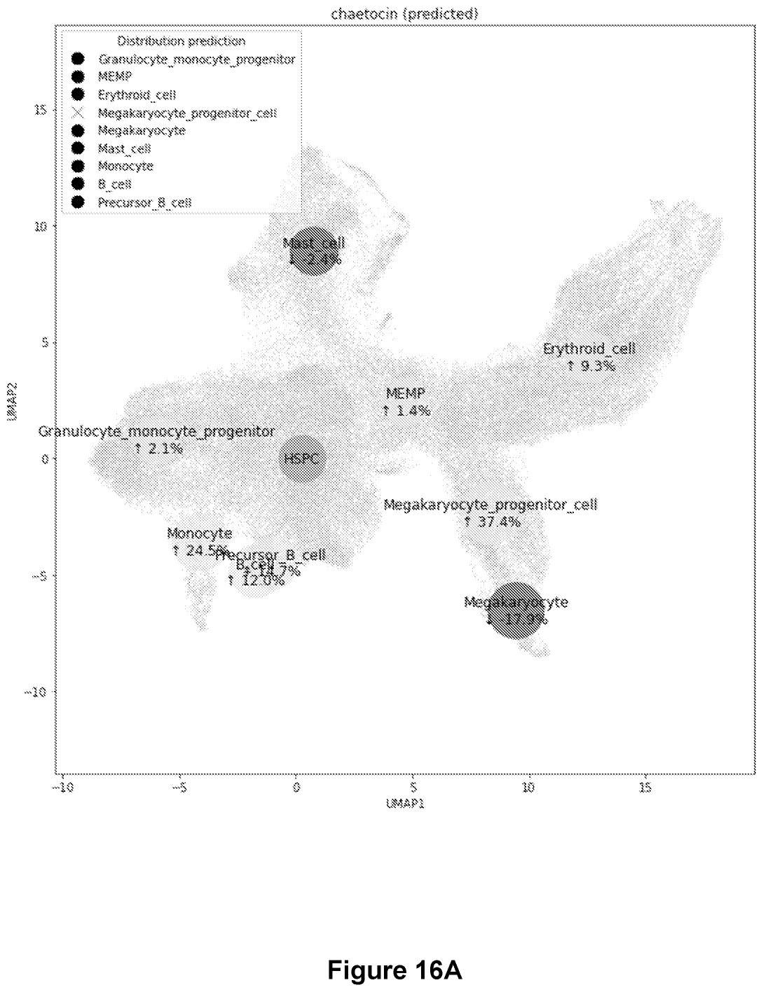

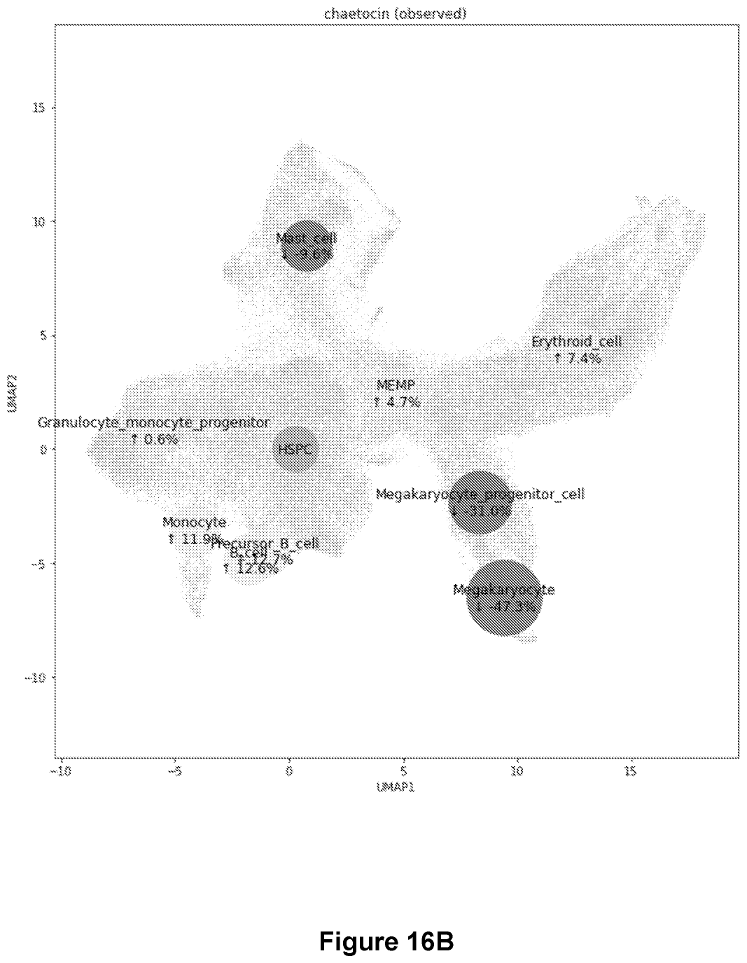

FIG. 16A illustrates the predicted UMAP plot (of increase or decrease in end cell types) provided by the model (e.g., in response to inputting into the model the fingerprint for chaetocin) while FIG. 16B illustrated the UMAP plot (of increase or decrease in end cell types) of actual measurement of increase or decrease in end cell types for in vitro CD34+ cells exposed to chaetocin. For information on chaetocin, see Isham et al., 2007, “Chaetocin: a promising new antimyeloma agent with in vitro and in vivo activity mediated via imposition of oxidative stress,” Blood 109, 2579, which is hereby incorporated by reference.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that includes a computer program mechanism embedded in a non-transitory computer readable storage medium. For instance, the computer program product could contain the program modules shown in any combination of FIG. 1, 2A-B, or 3. These program modules can be stored on a CD-ROM, DVD, magnetic disk storage product, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed is:

1. A method of training a model to identify a set of cellular constituents associated with a cellular process of interest, the method comprising:
at a computer system comprising a memory and one or more processors:
(A) obtaining one or more first datasets in electronic form, the one or more first datasets individually or collectively comprising:
for each respective cell in a first plurality of cells, wherein the first plurality of cells comprises twenty or more cells and collectively represents a plurality of annotated cell states:
for each respective cellular constituent in a plurality of cellular constituents, wherein the plurality of cellular constituents comprises 50 or more cellular constituents:
a corresponding abundance of the respective cellular constituent in the respective cell,
thereby accessing or forming a plurality of vectors, each respective vector in the plurality of vectors (i) corresponding to a respective cellular constituent in the plurality of constituents and (ii) comprising a corresponding plurality of elements, each respective element in the corresponding plurality of elements having a corresponding count representing the corresponding abundance of the respective cellular constituent in a respective cell in the first plurality of cells;
(B) using the plurality of vectors to identify each respective cellular constituent module in a plurality of cellular constituent modules, each respective cellular constituent module in the plurality of cellular constituent modules including an independent subset of the plurality of cellular constituents, wherein the plurality of cellular constituent modules are arranged in a latent representation dimensioned by (i) the plurality of cellular constituent modules and (ii) the plurality of cellular constituents or a representation thereof, and wherein the plurality of cellular constituent modules comprises more than ten cellular constituent modules;
(C) obtaining one or more second datasets in electronic form, the one or more second datasets individually or collectively comprising:
for each respective cell in a second plurality of cells, wherein the second plurality of cells comprises twenty or more cells and collectively represents a plurality of covariates associated with the cellular process of interest:
for each respective cellular constituent in the plurality of cellular constituents:
a corresponding abundance of the respective cellular constituent in the respective cell,
thereby obtaining a cellular constituent count data structure dimensioned by (i) the second plurality of cells and (ii) the plurality of cellular constituents or the representation thereof;
(D) forming an activation data structure by combining the cellular constituent count data structure and the latent representation using the plurality of cellular constituents or the representation thereof as a common dimension, wherein the activation data structure comprises, for each cellular constituent module in the plurality of cellular constituent modules:
for each cell in the second plurality of cells, a respective activation weight; and
(E) training the model using, for each respective covariate in the plurality of covariates, a difference between (i) a calculated activation against each cellular constituent module represented by the model upon input of a representation of the respective covariate into the model and (ii) actual activation against each cellular constituent module represented by the model, wherein the training adjusts a plurality of covariate parameters associated with the model responsive to the difference, wherein each respective covariate parameter in the plurality of covariate parameters represents a covariate in the plurality of covariates.

2. The method of claim 1, wherein the plurality of covariate parameters comprises, for each respective cellular constituent module in the plurality of cellular constituent modules, for each respective covariate, a corresponding parameter indicating whether the respective covariate correlates, across the second plurality of cells, with the respective cellular constituent module, and wherein the method further comprises:
(F) identifying, using the plurality of covariate weights upon training the model, one or more cellular constituent modules in the plurality of cellular constituent modules that is associated with one or more covariates in the plurality of covariates, thereby associating each cellular constituent in the set of plurality of cellular constituents, from among the cellular constituents in the identified one or more cellular constituent modules, with the cellular process of interest.

3. The method of claim 1 or 2, wherein an annotated cell state in the plurality of annotated cell states is an exposure of a cell in the first plurality of cells to a compound under an exposure condition.

4. The method of claim 3, wherein the exposure condition is a duration of exposure, a concentration of the compound, or a combination of a duration of exposure and a concentration of the compound.

5. The method of any one of claims 1-4, wherein each cellular constituent in the plurality of cellular constituents is a particular gene, a particular mRNA associated with a gene, a carbohydrate, a lipid, an epigenetic feature, a metabolite, a protein, or a combination thereof.

6. The method of any one of claims 1-4, wherein
each cellular constituent in the plurality of cellular constituents is a particular gene, a particular mRNA associated with a gene, a carbohydrate, a lipid, an epigenetic feature, a metabolite, a protein, or a combination thereof, and
the corresponding abundance of the respective cellular constituent in the respective cell in the first or second plurality of cells is determined by a colorimetric measurement, a fluorescence measurement, a luminescence measurement, or a resonance energy transfer (FRET) measurement.

7. The method of any one of claims 1-4, wherein
each cellular constituent in the plurality of cellular constituents is a particular gene, a particular mRNA associated with a gene, a carbohydrate, a lipid, an epigenetic feature, a metabolite, a protein, or a combination thereof, and
the corresponding abundance of the respective cellular constituent in the respective cell in the first or second plurality of cells is determined by single-cell ribonucleic acid (RNA) sequencing (scRNA-seq), scTag-seq, single-cell assay for transposase-accessible chromatin using sequencing (scATAC-seq), CyTOF/SCoP, E-MS/Abseq, miRNA-seq, CITE-seq, or any combination thereof.

8. The method of any one of claims 1-7, wherein using the plurality of vectors to identify each cellular constituent module in a plurality of cellular constituent modules comprises application of a correlation model to the plurality of vectors using each corresponding plurality of elements of each vector in the plurality of vectors.

9. The method of claim 8, wherein the correlation model includes a graph clustering.

10. The method of claim 9, wherein the graph clustering is Leiden clustering on a Pearson-correlation-based distance metric.

11. The method of claim 9, wherein the graph clustering is Louvain clustering.

12. The method of any one of claims 1-11, wherein using the plurality of vectors to identify each cellular constituent module in the plurality of cellular constituent modules comprises a dictionary learning model that produces the representation of the plurality of cellular constituents as a plurality of dimension reduction components.

13. The method of claim 12, wherein the dictionary learning model is L0-regularized autoencoder.

14. The method of any one of claims 1-13, wherein the plurality of cellular constituent modules consists of between 10 and 2000 cellular constituent modules.

15. The method of any one of claims 1-13, wherein the plurality of cellular constituent modules consists of between 50 and 500 cellular constituent modules.

16. The method of any one of claims 1-15, wherein the plurality of cellular constituents consists of between twenty and 10,000 cellular constituents.

17. The method of any one of claims 1-15, wherein the plurality of cellular constituents consists of between 100 and 8,000 cellular constituents.

18. The method of any one of claims 1-17, wherein each cellular constituent module in the plurality of constituent modules consists of between two cellular constituents and three hundred cellular constituents.

19. The method of any one of claims 1-18, wherein the cellular process of interest is an aberrant cell process associated with a disease, and the first plurality of cells includes cells that are representative of the disease and cells that are not representative of the disease as indicated by the plurality of annotated cell states.

20. The method of any one of claims 1-19, wherein a respective covariate in the plurality of covariates comprises cell batch and the representation of the respective covariate is a cell batch identification.

21. The method of any one of claims 1-19, wherein a respective covariate in the plurality of covariates comprises cell donor and the representation of the respective covariate is an identification of the cell donor or a characteristic of the cell donor.

22. The method of any one of claims 1-19, wherein a respective covariate in the plurality of covariates comprises cell type and the representation of the respective covariate is a cell type identification.

23. The method of any one of claims 1-19, wherein a respective covariate in the plurality of covariates comprises disease status and the representation of the respective covariate is an indication of absence or presence of the disease.

24. The method of any one of claims 1-19, wherein a respective covariate in the plurality of covariates comprises exposure to a compound and the representation of the respective covariate is a fingerprint of the compound.

25. The method of claim 24, the method further comprising generating the fingerprint from a chemical structure of the compound using Daylight, BCI, ECFP4, EcFC, MDL, TTFP, UNITY 2D, RNNS2S, GraphConv, fingerprint SMILES Transformer, RNNS2S, or GraphConv.

26. The method of claim 24 or 25, wherein the representation of the respective covariate further comprises a duration of time the respective covariate was incubated with the respective cell.

27. The method of any one of claims 24-26, wherein the representation of the respective covariate further comprises a concentration of the respective covariate used to incubate the respective cell.

28. The method of any one of claims 1-27, wherein the training the model (E) is performed using a categorical cross-entropy loss in a multi-task formulation, in which each covariate in the plurality of covariates corresponds to a cost function in plurality of cost functions and each respective cost function in the plurality of cost functions has a common weighting factor.

29. The method of any one of claims 1-28, the method further comprising using the identity of each cellular constituent in a cellular constituent module in the plurality of cellular constituent modules to associate the cellular constituent module with a known cellular pathway, a biological process, a transcription factor, a cell receptor, or a kinase using a contextualization algorithm.

30. The method of claim 29, wherein the contextualization algorithm is a gene set enrichment analysis algorithm.

31. The method of any one of claims 1-30, the method further comprising, for each respective cellular constituent module in the plurality of cellular constituent modules, using the identity of each cellular constituent in the respective cellular constituent module to associate the respective cellular constituent module with a known cellular pathway, a biological process, a transcription factor, a cell receptor, or a kinase using a contextualization algorithm.

32. The method of claim 31, wherein the contextualization algorithm is a gene set enrichment analysis algorithm.

33. The method of claim 31 or 32, the method further comprising pruning the activation data structure by removing from the activation data structure one or more cellular constituent modules that fail to associate with a known cellular pathway, a biological process, a transcription factor, a cell receptor, or a kinase that is implicated in the cellular process of interest.

34. The method of any one of claim 3, 4, or 24-27 wherein the compound is an organic compound having a molecular weight of less than 2000 Daltons.

35. The method of any one of claim 3, 4, or 24-27, wherein the compound is an organic compound that satisfies each of the Lipinski rule of five criteria.

36. The method of any one of claim 3, 4, or 24-27, wherein the compound is an organic compound that satisfies at least three criteria of the Lipinski rule of five criteria.

37. The method of any one of claim 1-13 or 16-36, wherein the plurality of cellular constituent modules comprises five or more cellular constituent modules.

38. The method of any one of claim 1-13 or 16-36, wherein the plurality of cellular constituent modules comprises ten or more cellular constituent modules.

39. The method of any one of claim 1-13 or 16-36, wherein the plurality of cellular constituent modules comprises 100 or more cellular constituent modules.

40. The method of any one of claims 1-39, wherein the independent subset of the plurality of cellular constituents in the respective cellular constituent module comprises five or more cellular constituents.

41. The method of any one of claims 1-39, wherein the independent subset of the plurality of cellular constituents in the respective cellular constituent module consists of between two and 20 cellular constituents in a molecular pathway associated with the cellular process of interest.

42. The method of any one of claim 1-7 or 14-41, wherein the model is a regressor.

43. The method of any one of claim 1-7 or 14-41, wherein the model is a logistic regression model, a neural network model, a support vector machine model, a Naive Bayes model, a nearest neighbor model, a boosted trees model, a random forest model, a decision tree model, a multinomial logistic regression model, a linear model, or a linear regression model.

44. The method of any one of claims 1-43, wherein each respective covariate parameter in the plurality of covariate parameters represents a different covariate in the plurality of covariates.

45. The method of any one of claims 1-44, wherein more than one covariate parameter in the plurality of covariate parameters represents a common covariate in the plurality of covariates.

46. The method of any one of claims 1-45, wherein a corresponding abundance of the respective cellular constituent in the respective cell is determined using a cell-based assay.

47. The method of any one of claims 1-46, wherein the first plurality of cells or the second plurality of cells comprises or consists of cells from an organ.

48. The method of claim 47, wherein the organ is heart, liver, lung, muscle, brain, pancreas, spleen, kidney, small intestine, uterus, or bladder.

49. The method of any one of claims 1-46, wherein the first plurality of cells or the second plurality of cells comprises or consists of cells from a tissue.

50. The method of claim 49, wherein the tissue is bone, cartilage, joint, tracheae, spinal cord, cornea, eye, skin, or blood vessel.

51. The method of any one of claims 1-46, wherein the first plurality of cells or the second plurality of cells comprises or consists of a plurality of stem cells.

52. The method of claim 51, wherein the plurality of stem cells is a plurality of embryonic stem cells, a plurality of adult stem cells, or a plurality of induced pluripotent stem cells (iPSC).

53. The method of any one of claims 1-46, wherein the first plurality of cells or the second plurality of cells comprises or consists of a plurality of primary human cells.

54. The method of claim 53, wherein the plurality of primary human cells are a plurality of CD34+ cells, a plurality of CD34+ hematopoietic stems, a plurality of progenitor cells (HSPC), a plurality of T-cells, a plurality of mesenchymal stem cells (MSC), a plurality of airway basal stem cells, or a plurality of induced pluripotent stem cells.

55. The method of any one of claims 1-46, wherein the first plurality of cells or the second plurality of cells comprises or consists of a plurality of human cell lines.

56. The method of any one of claims 1-46, wherein the first plurality of cells or the second plurality of cells comprises or consists of cells from umbilical cord blood, from peripheral blood, or from bone marrow.

57. The method of any one of claims 1-46, wherein the first plurality of cells or the second plurality of cells comprises or consists of cells in or from a solid tissue.

58. The method claim 57, wherein the solid tissue is placenta, liver, heart, brain, kidney, or gastrointestinal tract.

59. The method of any one of claims 1-46, wherein the first plurality of cells or the second plurality of cells comprises or consists of a plurality of differentiated cells.

60. The method of claim 59, wherein the plurality of differentiated cells is a plurality of megakaryocytes, a plurality of osteoblasts, a plurality of chondrocytes, a plurality of adipocytes, a plurality of hepatocytes, a plurality of hepatic mesothelial cells, a plurality of biliary epithelial cells, a plurality of hepatic stellate cells, a plurality of hepatic sinusoid endothelial cells, a plurality of Kupffer cells, a plurality of pit cells, a plurality of vascular endothelial cells, a plurality of pancreatic duct epithelial cells, a plurality of pancreatic duct cells, a plurality of centroacinous cells, a plurality of acinar cells, a plurality of islets of Langerhans, a plurality of cardiac muscle cells, a plurality of fibroblasts, a plurality of keratinocytes, a plurality of smooth muscle cells, a plurality of type I alveolar epithelial cells, a plurality of type II alveolar epithelial cells, a plurality of Clara cells, a plurality of ciliated epithelial cells, a plurality of basal cells, a plurality of goblet cells, a plurality of neuroendocrine cells, a plurality of kultschitzky cells, a plurality of renal tubular epithelial cells, a plurality of urothelial cells, a plurality of columnar epithelial cells, a plurality of glomerular epithelial cells, a plurality of glomerular endothelial cells, a plurality of podocytes, a plurality of mesangium cells, a plurality of nerve cells, a plurality of astrocytes, a plurality of microglia, or a plurality of oligodendrocytes.

61. The method of claim 2, wherein the set of cellular constituents consists of between 2 and 20 cellular constituents in the plurality of cellular constituent and the one or more cellular constituent modules consists of a single cellular constituent module.

62. The method of claim 2, wherein the set of cellular constituents consists of between 2 and 100 cellular constituents in the plurality of cellular constituent and the one or more cellular constituent modules comprises two or more cellular constituent modules.

63. The method of claim 2, wherein the set of cellular constituents consists of between 2 and 1000 cellular constituents in the plurality of cellular constituent and the one or more cellular constituent modules comprises five or more cellular constituent modules.

64. The method of any one of claims 1-63, wherein the model is an ensemble model comprising a plurality of component models, and wherein each respective component model in the plurality of component models provides a calculated activation for a different cellular constituent module in the plurality of cellular constituent modules responsive to inputting the representation of the respective covariate into the respective component model.

65. The method of claim 64, wherein the ensemble model includes a different component model for each cellular constituent module in the plurality of cellular constituent modules.

66. The method of claim 64, wherein a component model in the plurality of component models is a logistic regression model, a neural network model, a support vector machine model, a Naive Bayes model, a nearest neighbor model, a boosted trees model, a random forest model, a decision tree model, a multinomial logistic regression model, a linear model, or a linear regression model.

67. A computer system, comprising one or more processors and memory, the memory storing instructions for performing a method for training a model to identify a set of cellular constituents associated with a cellular process of interest, the method comprising:
(A) obtaining one or more first datasets in electronic form, the one or more first datasets individually or collectively comprising:
for each respective cell in a first plurality of cells, wherein the first plurality of cells comprises twenty or more cells and collectively represents a plurality of annotated cell states:
for each respective cellular constituent in a plurality of cellular constituents, wherein the plurality of cellular constituents comprises 50 or more cellular constituents:
a corresponding abundance of the respective cellular constituent in the respective cell,
thereby accessing or forming a plurality of vectors, each respective vector in the plurality of vectors (i) corresponding to a respective cellular constituent in the plurality of constituents and (ii) comprising a corresponding plurality of elements, each respective element in the corresponding plurality of elements having a corresponding count representing the corresponding abundance of the respective cellular constituent in a respective cell in the first plurality of cells;
(B) using the plurality of vectors to identify each respective cellular constituent module in a plurality of cellular constituent modules, each respective cellular constituent module in the plurality of cellular constituent modules including an independent subset of the plurality of cellular constituents, wherein the plurality of cellular constituent modules are arranged in a latent representation dimensioned by (i) the plurality of cellular constituent modules and (ii) the plurality of cellular constituents or a representation thereof, and wherein the plurality of cellular constituent modules comprises more than ten cellular constituent modules;
(C) obtaining one or more second datasets in electronic form, the one or more second datasets individually or collectively comprising:
for each respective cell in a second plurality of cells, wherein the second plurality of cells comprises twenty or more cells and collectively represents a plurality of covariates associated with the cellular process of interest:
for each respective cellular constituent in the plurality of cellular constituents:
a corresponding abundance of the respective cellular constituent in the respective cell,
thereby obtaining a cellular constituent count data structure dimensioned by (i) the second plurality of cells and (ii) the plurality of cellular constituents or the representation thereof;
(D) forming an activation data structure by combining the cellular constituent count data structure and the latent representation using the plurality of cellular constituents or the representation thereof as a common dimension, wherein the activation data structure comprises, for each cellular constituent module in the plurality of cellular constituent modules:
for each cell in the second plurality of cells, a respective activation weight; and
(E) training the model using, for each respective covariate in the plurality of covariates, a difference between (i) a calculated activation against each cellular constituent module represented by the model upon input of a representation of the respective covariate into the model and (ii) actual activation against each cellular constituent module represented by the model, wherein the training adjusts a plurality of covariate parameters associated with the model responsive to the difference, wherein each respective covariate parameter in the plurality of covariate parameters represents a covariate in the plurality of covariates.

68. The computer system of claim 67, wherein the plurality of covariate parameters comprises, for each respective cellular constituent module in the plurality of cellular constituent modules, for each respective covariate, a corresponding parameter indicating whether the respective covariate correlates, across the second plurality of cells, with the respective cellular constituent module, and wherein the method further comprises:
(F) identifying, using the plurality of covariate weights upon training the model, one or more cellular constituent modules in the plurality of cellular constituent modules that is associated with one or more covariates in the plurality of covariates, thereby associating each cellular constituent in the set of plurality of cellular constituents, from among the cellular constituents in the identified one or more cellular constituent modules, with the cellular process of interest.

69. A non-transitory computer-readable medium storing one or more computer programs, executable by a computer, a method for training a model to identify a set of cellular constituents associated with a cellular process of interest, the computer comprising one or more processors and a memory, the one or more computer programs collectively encoding computer executable instructions that perform a method comprising:
(A) obtaining one or more first datasets in electronic form, the one or more first datasets individually or collectively comprising:
for each respective cell in a first plurality of cells, wherein the first plurality of cells comprises twenty or more cells and collectively represents a plurality of annotated cell states:
for each respective cellular constituent in a plurality of cellular constituents, wherein the plurality of cellular constituents comprises 50 or more cellular constituents:
a corresponding abundance of the respective cellular constituent in the respective cell,
thereby accessing or forming a plurality of vectors, each respective vector in the plurality of vectors (i) corresponding to a respective cellular constituent in the plurality of constituents and (ii) comprising a corresponding plurality of elements, each respective element in the corresponding plurality of elements having a corresponding count representing the corresponding abundance of the respective cellular constituent in a respective cell in the first plurality of cells;
(B) using the plurality of vectors to identify each respective cellular constituent module in a plurality of cellular constituent modules, each respective cellular constituent module in the plurality of cellular constituent modules including an independent subset of the plurality of cellular constituents, wherein the plurality of cellular constituent modules are arranged in a latent representation dimensioned by (i) the plurality of cellular constituent modules and (ii) the plurality of cellular constituents or a representation thereof, and wherein the plurality of cellular constituent modules comprises more than ten cellular constituent modules;
(C) obtaining one or more second datasets in electronic form, the one or more second datasets individually or collectively comprising:
for each respective cell in a second plurality of cells, wherein the second plurality of cells comprises twenty or more cells and collectively represents a plurality of covariates associated with the cellular process of interest:
for each respective cellular constituent in the plurality of cellular constituents:
a corresponding abundance of the respective cellular constituent in the respective cell,
thereby obtaining a cellular constituent count data structure dimensioned by (i) the second plurality of cells and (ii) the plurality of cellular constituents or the representation thereof;
(D) forming an activation data structure by combining the cellular constituent count data structure and the latent representation using the plurality of cellular constituents or the representation thereof as a common dimension, wherein the activation data structure comprises, for each cellular constituent module in the plurality of cellular constituent modules:
for each cell in the second plurality of cells, a respective activation weight; and
(E) training the model using, for each respective covariate in the plurality of covariates, a difference between (i) a calculated activation against each cellular constituent module represented by the model upon input of a representation of the respective covariate into the model and (ii) actual activation against each cellular constituent module represented by the model, wherein the training adjusts a plurality of covariate parameters associated with the model responsive to the difference, wherein each respective covariate parameter in the plurality of covariate parameters represents a covariate in the plurality of covariates.

70. The non-transitory computer-readable medium of claim 69, wherein the plurality of covariate parameters comprises, for each respective cellular constituent module in the plurality of cellular constituent modules, for each respective covariate, a corresponding parameter indicating whether the respective covariate correlates, across the second plurality of cells, with the respective cellular constituent module, and wherein the method further comprises:
(F) identifying, using the plurality of covariate weights upon training the model, one or more cellular constituent modules in the plurality of cellular constituent modules that is associated with one or more covariates in the plurality of covariates, thereby associating each cellular constituent in the set of plurality of cellular constituents, from among the cellular constituents in the identified one or more cellular constituent modules, with the cellular process of interest.

71. A method of associating a plurality of cellular constituents with a cellular process of interest, the method comprising:
at a computer system comprising a memory and one or more processors:
(A) obtaining one or more first datasets in electronic form, the one or more first datasets individually or collectively comprising:
for each respective cell in a first plurality of cells, wherein the first plurality of cells comprises twenty or more cells and collectively represents a plurality of annotated cell states:
for each respective cellular constituent in a plurality of cellular constituents, wherein the plurality of cellular constituents comprises 50 or more cellular constituents:
a corresponding abundance of the respective cellular constituent in the respective cell,
thereby accessing or forming a plurality of vectors, each respective vector in the plurality of vectors (i) corresponding to a respective cellular constituent in the plurality of constituents and (ii) comprising a corresponding plurality of elements, each respective element in the corresponding plurality of elements having a corresponding count representing the corresponding abundance of the respective cellular constituent in a respective cell in the first plurality of cells;
(B) using the plurality of vectors to identify each cellular constituent module in a plurality of cellular constituent modules, each cellular constituent module in the plurality of cellular constituent modules including a subset of the plurality of cellular constituents, and wherein the plurality of cellular constituent modules comprises more than ten cellular constituent modules;
(C) for each respective cellular constituent module in the plurality of cellular constituent modules, using the identity of each cellular constituent in the respective cellular constituent module to associate the respective cellular constituent module with a known cellular pathway, a biological process, a transcription factor, a cell receptor, or a kinase using a contextualization algorithm; and
(D) pruning the activation data structure by removing from the activation data structure one or more cellular constituent modules that fail to associate with a known cellular pathway, a biological process, a transcription factor, a cell receptor, or a kinase that is implicated in the cellular process of interest thereby identifying one or more cellular constituent modules in the plurality of cellular constituent modules that is associated with the cellular process of interest and, from the one or more cellular constituent modules, the plurality of cellular constituents associated with the cellular process of interest.

72. The method of claim 71, wherein the contextualization algorithm is a gene set enrichment analysis algorithm.
